(19) United States
(12) Patent Application Publication
(10) Pub. No.: US 2012/0122848 A1
(43) Pub. Date: May 17, 2012

(54) (+)-3-HYDROXYMORPHINAN DERIVATIVES AS NEUROPROTECTANTS

(75) Inventors: Jinhwa Lee, Yongin-si (KR); Jong Yup Kim, Yongin-si (JP); Kwang-Seop Song, Yongin-si (KR); Jeongmin Kim, Yongin-si (KR); Kwang Woo Ahn, Yongin-si (KR); Yonggyu Kong, Yongin-si (KR)

(73) Assignee: GREEN CROSS CORPORATION, Yongin-si, Gyeonggi-do (KR)

(21) Appl. No.: 13/387,563

(22) PCT Filed: Jul. 28, 2010
(86) PCT No.: PCT/KR10/04959

§ 371 Date: Jan. 27, 2012

Related U.S. Application Data
(60) Provisional application No. 61/229,550, filed on Jul. 29, 2009.

(51) Int. Cl. A61K 31/485; C07D 491/08; C07D 401/10; C07D 405/12; A61P 25/16; A61K 31/55; A61K 31/496; A61K 31/5377; A61P 25/28; C07D 221/28; C07D 413/10

(52) U.S. Cl. 514/217.07; 546/74; 546/44; 540/597; 544/361; 544/125; 514/289; 514/282; 514/253.02; 514/232.8

(57) ABSTRACT

A novel (+)-3-hydroxymorphinan derivatives and a pharmaceutical composition comprising the same as an active ingredient, which are useful for preventing or treating a neurodegenerative disease, are provided.

### FIELD OF THE INVENTION

The present invention relates to (+)-3-hydroxymorphinan derivatives which are effective as neuroprotectants.

### BACKGROUND OF THE INVENTION

The concept of neuroprotection was applied to chronic diseases of the brain as well as acute neurological conditions, since some of the basic mechanisms of damage to the central nervous system (CNS) are similar in these conditions. Neurodegenerative disorders include Parkinson's disease (PD), Alzheimer's disease (AD), Huntington's disease (HD), and amyotrophic lateral sclerosis (ALS). Neuroprotection has been regarded to be the mechanism of action of some of the drugs used in the treatment of these conditions.

Neurodegeneration in PD, AD, and other neurodegenerative diseases seems to be multifactorial, in that a complex set of toxic reactions including inflammation, glutamatergic neurotoxicity, increases in iron and nitric oxide, depletion of endogenous antioxidants, reduced expression of trophic factors, dysfunction of the ubiquitin-proteasome system, and expression of proapoptotic proteins leads to the death of neurons. Gangliosides are the major class of glycoconjugates on neurons and carry the majority of the sialic acid within the CNS. Ganglioside synthesis is essential for the development of a stable CNS. Interruption of ganglioside synthesis produces CNS degeneration and modified axon-glial interactions [Yamashita, T. et al., PNAS, 2005, 102, 2725-2730]. Thus, the fundamental objective in neurodegeneration and neuroprotection research is to determine which of these factors embodies the primary event, the sequence in which these events occur, and whether they act in concurrence in the pathogenic process. This has resulted in the concept that drugs addressed against a single target will be ineffective and instead a single drug with multiple pharmacological properties or a cocktail of drugs may be more appropriate. Among the many factors involved, apoptosis and glutamate toxicity play an important role.

Apoptosis mediated by genetic programs intrinsic to the cell is being implicated in neurodegenerative disorders. During the normal development of the vertebrate nervous system, approximately 50% of the different types of neurons usually die right after they establish synaptic connections with their target cells. It has been hypothesized that this death is due to failure of these neurons to obtain adequate amounts of survival specific neurotropic factors from target cells. The mechanism of death is postulated to be deprival of extracellular survival signals, which normally suppress apoptosis.

Many neurodegenerative disorders are distinguished by conformational alteration in proteins that result in misfolding, aggregation, and intra- or extra-neuronal accumulation of amyloid fibrils. Molecular chaprones provide a first line of defence against misfolded, aggregation-prone proteins and are among the most potent suppressors of neurodegeneration known for animal models of human disease. A better understanding of the molecular basis of chaperon-mediated protection against neurodegeneration may result in the development of therapies for neurodegenerative disorders that are associated with protein misfolding and aggregation.

There are approximately 100 million people in the world and 800,000 people in the United States alone with Parkinson's disease (PD).

Parkinson's disease is a result of chronic progressive degeneration of neurons, the cause of which has not yet completely been clarified. While the primary cause of Parkinson's disease is not known, it is characterized by degeneration of dopaminergic neurons of the substantia nigra (SN). The substantia nigra is a portion of the lower brain, or brain stem that helps control voluntary movements. The shortage of dopamine in the brain caused by the loss of these neurons is believed to cause the observable disease symptoms. Clinically, it manifests in the form of the cardinal symptoms resting tremors, rigor, bradykinesia, and postural instability.

Levodopa, dopamine agonists (e.g., rotigotine, pramipexol, bromocriptine, ropinirol, cabergoline, pergolide, apomorphine and lisuride), anticholinergics, NMDA antagonists, β-blocker as well as the MAO-B inhibitor selegiline and the COMT inhibitor entacapone are used as medicines for relief from the motor symptoms. Most of these agents intervene in the dopamine and/or choline signal cascade and thereby symptomatically influence the Parkinson-typical movement disorders.

In the present therapy for the Parkinson's disease, treatment is initiated after the appearance of the cardinal symptoms. In general, Parkinson's disease is said to be clinically evident if at least two of the four cardinal symptoms (bradykinesia, resting tremors, rigor, and postural instability) are detected and respond to L-dopa [Hughes, J Neurol Neurosurg Psychiatry, 1992, 55, 181]. Unfortunately, the motor function disorders in Parkinson's disease patients become apparent only after about 70-80% of the dopaminergic neurons in the substantia nigra (SN) are irreparably damaged [Becker et al., J Neurol 249, 2002, Suppl 3:III, 40; Hornykiewicz, Encyclopaedia of Life Science 2001, 1]. Chances of a therapy with lasting effects are very bleak at that point. Hence, it is desirable to initiate the therapy as early as possible.

Current clinical observations as well as anatomical and genetic research show that diagnosis of Parkinson patients at an early stage and identification of high risk patients is possible. With that an opportunity arises for influencing the disease process at a point of time when more neurons are still there, rather than at the time of appearance of several cardinal motor symptoms of the Parkinson's disease, and thereby for protecting a quantitatively greater number of neurons. One can expect that the administration of an effective neuroprotective agent at an early stage will significantly delay the process of the development of the disease. The sooner the therapy is initiated, the higher are the chances of a long lasting prevention of the onset of symptoms, which degrade the quality of life.

Hence, such remedies are needed that not only influence the dopaminergic transmission and alleviate the symptoms of the Parkinson's disease in advanced stages, but also reverse, prevent, or at least significantly delay the dopaminergic neuron extinction in the early, to a great extent motor-asymptomatic, Parkinson stages [Dawson, Nat. Neurosci. Supplement, 5, 2002, 1058].

Alzheimer's disease (AD) is a progressive degenerative disorder of the brain that begins with memory impairment and eventually progresses to dementia, physical impairment, and death. Patients develop various psychiatric and neurological signs during the course of the disease. The prevalence rates of dementia vary significantly in different countries, but range from 2.1% to 10.5%. AD is the most common type of dementia. Several factors play a role in the etiology and pathogenesis of AD: aging; genetic risk factors; amyloid precursor protein and beta-amyloid accumulation; tau hyperphosphorylation; membrane disturbances, phospholipid metabolism, and disruption of signal transduction; inflammatory reactions and immunological disturbances; environmental toxins; neurotransmitter defects and imbalances; neuroendocrine disturbances; oxidative injury; and free radicals, etc. AD is certainly not the result of a single operative mechanism but more likely comprises one or more processes that lead to intrinsic neuronal cell killing. A complex disease like AD is difficult to attack because no single approach is adequate and the development of a single universal therapy is unlikely. The most distinctive finding in the brains of patients with AD is copious deposits of amyloid β (Aβ). Aβ is found in small quantities in normal brains. Amyloid deposits by themselves do not damage the brain, but in the presence of apoE, amyloid forms into hair-shaped fibrils, and neuritic plaques [Holtzman, D. M. et al. PNAS, 2000, 97, 2892-2897]. The fact that apoE4 can increase both the amount of A and the formation of amyloid fibrils seems to indicate that this version of the lipoprotein is a genetic risk factor for AD.

Current therapies involving cholinesterase inhibitors such as rivastigmine, donepezil and galantamine are not considered neuroprotective. These drugs act to increase brain acetylcholine and offset aspects of the cognitive decline during early stages of the disease. The efficacy of these compounds is modest and short-lived as the disease progresses. Since multiple mechanisms are involved in the pathogenesis of AD, current therapies target one of the several disturbances in AD. Free radical scavengers address at eliminating only one type of disturbance. One of the problems in designing reasonable therapies is dissent on the cellular events that elicit brain-cell death in AD and lead to dementia. One view is that amyloid plaques, composed mostly of the amyloid protein, accumulate outside of brain neurons, growing larger and larger until they rupture the cells and kill them. Another view is that neurofibrillary tangles kill the cell. Some of the therapies related to neuroprotection include anti-inflammatory drugs, calcium channel blockers, antioxidants, glutamate antagonists, or inhibition of amyloid plaque formation.

The sirtuins are a family of enzymes which control diverse and virtual cellular functions, including metabolism and aging. Manipulations of sirtuin activities cause activation of anti-apoptotic, anti-inflammatory, anti-stress responses, and the modulation of an aggregation of proteins involved in neurodegenerative disorders. Recently, sirtuins were found to be disease-modifiers in various models of neurodegeneration. However, almost in all instances, the exact mechanisms of neuroprotection remain elusive. Nonetheless, the engineering of sirtuin activities is attractive as a novel therapeutic strategy for the treatment of currently neurodegenerative disorders such as AD and PD. There is a review article showing current data which support the putative therapeutic roles of sirtuin in aging and in neurodegenerative diseases and the feasibility of the development of sirtuin-based therapies [Kazantsev, A. et al. Biochim. Biophys. Acta, 2008, 1782, 363-369]. According to a literature, resveratrol, which is known to extend lifespan, improves mitochondrial function and protects against metabolic disease by activating SirT1 and PGC-1 [Lagouse, M. et al. Cell, 2006, 127, 1109-1122]. Another article reported that expression of SirT1 may be a good sensor of toxic neuronal processes, such as aging or neurodegenerative processes [Pallas, M. et al. Neurosci. 2008, 154, 1388-1397].

(+)-3-Hydroxymorphinan ((+)-3-HM) and its derivatives have shown the neuroprotective property in 1-methyl-4-phenyl-1,2,3,6-tetrahydropyridine (MPTP) models for PD. In this animal model, daily injections with (+)-3-HM or its analogs showed that dopamine (DA) neurons in substantia nigra pars compacta have been protected and DA levels in striatum has been restored (US Patent Publication No. 2005-0256147 A1; International Patent Publication No. WO2005/110412; Zhang et al. FASEB J. 2006 Dec. 20(14):2496-2511; Zhang et al. FASEB J. 2005 Mar. 19(3):395-397; and Kim et al. Life Sci. 72 (2003) 1883-1895). However, (+)-3-HM and its derivatives are efficacious only if they are administered intraperitoneally or intravenously. The previous invention of our laboratories [Green Cross Corp., WO 2008/111767 (2008)] relates to an orally bioavailable, novel prodrug of (+)-3-hydroxymorphinan which is effective as a neuroprotective agent for PD, when they are delivered orally. On the other hand, the present invention relates to novel (+)-3-HM derivatives which are effective as a neuroprotective agent in pharmacotherapy for neurodegenerative disease including AD, PD, Huntington's disease (HD) and amyotrophic lateral sclerosis (ALS).

### SUMMARY OF THE INVENTION

It is a primary object of the present invention to provide a novel (+)-3-hydroxymorphinan derivative of formula (I), or a pharmaceutically acceptable salt or a prodrug thereof, which is effective as a neuroprotective agent for a neurodegenerative disease.

It is another object of the present invention to provide a method for preparing the compound.

It is a further object of the present invention to provide a pharmaceutical composition comprising the compound as an active ingredient for treating or preventing a neurodegenerative disease including AD, PD, Huntington's disease (HD), and amyotrophic lateral sclerosis (ALS).

In accordance with an aspect of the present invention, there is provided a compound of formula (I), or a pharmaceutically acceptable salt or a prodrug thereof:

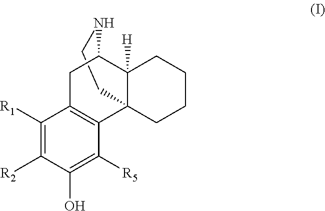

wherein,

R₁ is selected from the group consisting of hydrogen, C₁-C₃ alkyl, C₃-C₅ cycloalkyl, and halogen;

R₂ is selected from the group consisting of hydrogen; hydroxyl; mercapto; sulfanyl; sulfonyl; formyl; carboxyl; —NR₃R₄; halogen; C₁-C₁₀ alkyl; C₁-C₁₀ alkoxy; C₃-C₇ cycloalkyl; heterocycloalkyl; aryl; heteroaryl; —C₁-C₄ alkyl-Ar; and C₁-C₁₀ alkyl, C₃-C₇ cycloalkyl, heterocycloalkyl, aryl, heteroaryl, and —C₁-C₄ alkyl-Ar substituted with one or more Z groups, Ar being selected from the group consisting of phenyl, naphthyl, furyl, pyridyl, thienyl, thiazolyl, isothiazolyl, pyrazolyl, triazolyl, tetrazolyl, imidazolyl, imidazolidinyl, benzofuranyl, indolyl, thiazolidinyl, isoxazolyl, oxadiazolyl, thiadiazolyl, morpholinyl, piperidinyl, piperazinyl, pyrrolyl, and pyrimidinyl, and Z being independently selected from the group consisting of hydroxyl, C₁-C₄ alkyl, C₁-C₄ alkoxy, —(CH₂)ₘC(O)OR₃, —C(O)NR₃R₄, —CN, —(CH₂)ₙOH, —NO₂, F, Cl, Br, I, —NR₃R₄ and —NHC(O)R₃, wherein m is 0 to 4, and n is 0 to 4;

R₃ and R₄ are independently selected from the group consisting of hydrogen; C₁-C₆ alkyl; C₁-C₆ alkyl substituted with one or two R₇ groups; C₁-C₆ alkoxy; C₃-C₆ cycloalkyl; heterocycloalkyl; phenyl; heteroaryl; and C₃-C₆ cycloalkyl, heterocycloalkyl, phenyl, and heteroaryl substituted with one to three R₆ groups; or R₃ and R₄ are joined together with the N-atom to which they are attached, forming a heterocycloalkyl group or a heterocycloalkyl group substituted with one to three R₆ groups;

each R₆ is independently selected from the group consisting of hydroxyl; C₁-C₆ alkyl; C₁-C₆ alkyl substituted with one to three R₇ groups; C₁-C₆ alkoxy; halo(C₁-C₆ alkoxy); C₃-C₆ cycloalkyl; C₃-C₆ cycloalkyl substituted with one —NRₐR_{b} or pyrrolidinyl; heterocycloalkyl; phenyl; heteroaryl; —C(O)NRₐR_{b}; —C(O)R_{c}; —C(O)OR_{c}; oxo; cyano; —NRₐR_{b}; halogen; (C₁-C₆ alkyl)ureido, arylureido, and (C₁-C₆ alkylthio)ureido;

each R₇ is independently selected from the group consisting of hydroxyl; C₁-C₃ alkoxy; halogen; phenyl; cyano; —NRₐR_{b}; —C(O)NRₐR_{b}; —C(O)R_{c}; C₃-C₆ cycloalkyl; C₃-C₆ cycloalkyl substituted with one hydroxyl, heterocycloalkyl or —NRₐR_{b} group; heterocycloalkyl; heteroaryl; and heteroaryl substituted with one methyl, —NRₐR_{b} or hydroxyl;

each Rₐ is independently selected from the group consisting of hydrogen; C₁-C₃ alkyl; and C₁-C₃ alkyl substituted with one hydroxyl, methoxy, or dimethylamine; (C₁-C₄ alkyl)sulfonyl; arylsulfonyl: (C₁-C₄ alkyl)carbonyl; and (C₁-C₄ alkoxy)carbonyl;

each R_{b} is independently selected from the group consisting of hydrogen and C₁-C₃ alkyl;

each R_{c} is independently selected from the group consisting of hydrogen; C₁-C₃ alkyl; C₁-C₃ alkyl substituted with one methoxy group; phenyl; heterocycloalkyl; and heteroaryl; and

R₅ is selected from the group consisting of hydrogen and halogen, or R₅ is joined together with the adjacent hydroxyl group to form a heterocycloaryl group having two oxygens.

In accordance with another aspect of the present invention, there is provided a method for preparing a compound of formula (I) comprising the steps of:

subjecting (+)-3-hydroxymorphinan HBr salt to an amino protecting reaction to obtain a compound of formula (II);

conducting an electrophilic fluorination of the compound of formula (II) to obtain a 2-fluoro analogue thereof; treating the compound of formula (II) with 1-hydroxy-1,2-benziodoxol-3(1H)-one 1-oxide, followed by reduction to provide corresponding o-diphenol derivatives; or conducting an electrophilic iodination of the compound of formula (II), followed by treating with NaOMe; and

carrying out hydrogenation of the resulting compound using palladium catalyst,

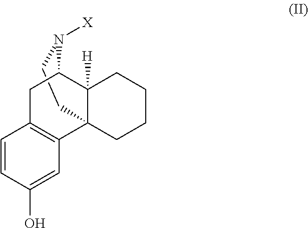

wherein, X is an amino protecting group.

The compound of formula (I) may also be prepared by a method comprising the steps of subjecting the o-diphenol derivative to a reaction with diiodomethane in the presence of a base, followed by a hydrogenation using palladium catalyst.

In accordance with another aspect of the present invention, there is provided a method for preparing a compound of formula (I) comprising the steps of:

conducting a selective ortho-formylation of the compound of formula (II) to obtain a compound of formula (III); and

subjecting the compound of formula (III) to hydrogenation using palladium catalyst, oxidation, fluorination using a fluorinating agent, or reduction using a reducing agent, followed by hydrogenation using palladium catalyst,

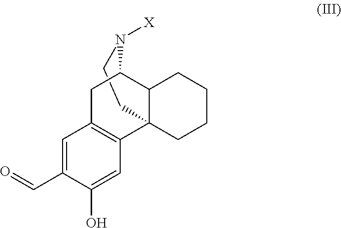

wherein, X is an amino protecting group.

In accordance with another aspect of the present invention, there is provided a method for preparing a compound of formula (I) comprising the steps of:

subjecting (+)-3-hydroxymorphinan HBr salt to iodination to obtain (+)-2-iodo-3-hydroxymorphinan;

introducing an amino protecting group to (+)-2-iodo-3-hydroxymorphinan to obtain a compound of formula (IV);

conducting methylation of the compound of formula (IV) to obtain a compound of formula (V);

subjecting the compound of formula (V) to coupling reaction with cyclic amine, aniline, alkylamine, or thiol, or subjecting the compound of formula (V) to palladium-catalyzed Suzuki-Miyaura cross-coupling reaction with arylboronic acid or alkylboronic acid to obtain a compound of formula (VI);

carrying out demethylation of the compound of formula (VI),

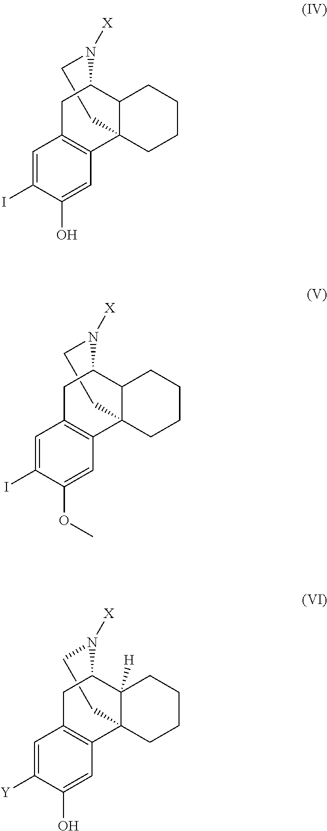

wherein, X is an amino protecting group, and Y is selected from the group consisting of —NR₃R₄; piperidinyl; mercapto; sulfanyl; aryl; C₁-C₁₀ alkyl; and piperidinyl, aryl and C₁-C₁₀ alkyl substituted with one or more Z groups, R₃, R₄ and Z having the same meanings as defined above.

In accordance with another aspect of the present invention, there is provided a method for preparing a compound of formula (I) comprising the steps of:

neutralizing (+)-3-hydroxymorphinan HBr salt with a hydroxide of alkali metal to obtain (+)-3-hydroxymorphinan;

treating (+)-3-hydroxymorphinan with HNO₃ to obtain 2-nitro-3-hydroxymorphinan;

introducing an amino protecting group to 2-nitro-3-hydroxymorphinan to obtain a compound of formula (VII);

carrying out methylation of the compound of compound of formula (VII) to obtain a compound of formula (VIII);

reducing the compound of formula (VIII) to the compound of formula (IX);

subjecting the compound of formula (IX) to reation with 2-chloroethyl ether in the presence of a base, or reductive alkylation with aldehyde or ketone; or conducting amino protection reaction, alkylation, deprotection, and reductive alkylation of the compound of formula (IX) successively to obtain a compound of formula (X); and

carrying out demethylation of the compound of formula (X),

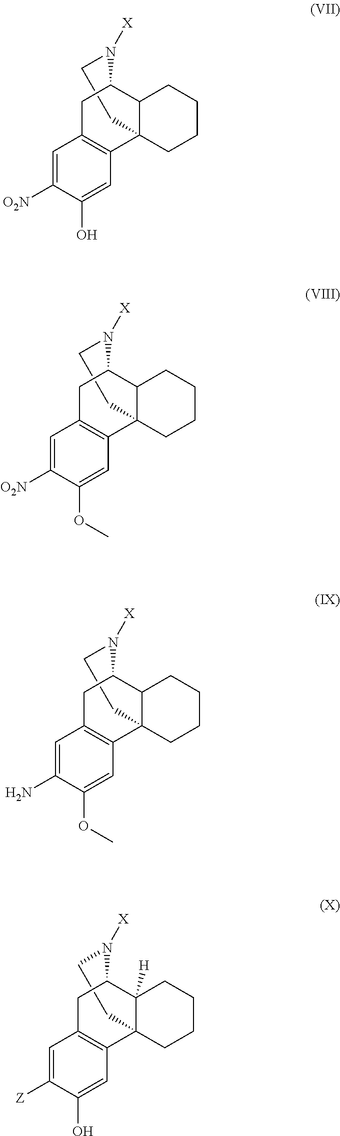

wherein, X is an amino protecting group and Z is 4-morpholinyl or —NR₃R₄, R₃ and R₄ having the same meanings as defined above.

In accordance with another aspect of the present invention, there is provided a method for preparing a compound of formula (I) comprising the step of subjecting (+)-3-hydroxymorphinan HBr salt to a reaction with tertiary alcohol, neutralization, or bromination using bromine.

In accordance with another aspect of the present invention, there is provided a pharmaceutical composition for preventing or treating a neurodegenerative disease, which comprises the compound of formula (I), or the pharmaceutically acceptable salt or a prodrug thereof as an active ingredient, and a pharmaceutically acceptable carrier.

In accordance with a further aspect of the present invention, there is provided a method for preventing or treating a neurodegenerative disease, which comprises administering the compound of formula (I), or the pharmaceutically acceptable salt or a prodrug thereof to a mammal in need thereof.

### BRIEF DESCRIPTION OF DRAWINGS

The above and other objects and features of the present invention will become apparent from the following description of the invention taken in conjunction with the following accompanying drawings, which respectively show:

FIG. 1: a graph showing the results of reactive oxygen species (ROS) measurement of the compound of Example 26; and

FIGS. 2A and 2B: graphs showing the results of reverse transcription-polymerase chain reaction of the compound of Example 26;

FIG. 3: a graph showing the results of western blotting analysis of the compound of Example 26; and

FIG. 4: a graph showing the results of total antioxidant activity assay of the compound of Example 26.

### DETAILED DESCRIPTION OF THE INVENTION

Hereinafter, a detailed description of the present invention is given.

As used herein, the term “alkyl” refers to a straight or branched chain saturated hydrocarbon radical. Examples of “alkyl” as used herein include, but are not limited to, methyl, ethyl, n-propyl, isopropyl, n-butyl, isobutyl, t-butyl, n-pentyl, isopentyl and hexyl.

As used herein, the term “cycloalkyl” refers to a non-aromatic cyclic hydrocarbon radical composed of three to seven carbon atoms. Exemplary “cycloalkyl” groups include, but are not limited to, cyclopropyl, cyclobutyl, cyclopentyl, cyclohexyl and cycloheptyl.

As used herein, the term “heterocycloalkyl” refers to a three to seven-membered hydrocarbon ring containing one or more heteroatomic moieties selected from S, SO, SO₂, O, N, or N-oxide, optionally substituted with one or more substituents selected from the group which includes substituted C₁₋₃ alkyl, substituted C₂₋₃ alkenyl, substituted C₂₋₃ alkynyl, heteroaryl, heterocyclic, aryl, C₁₋₃ alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen, and ureido. Such a ring can be saturated or have one or more degrees of unsaturation. Such a ring may be optionally fused to one or more “heterocyclic” ring(s), aryl ring(s), heteroaryl ring(s) or carbocycle ring(s), each having optional substituents.

Examples of “heterocycloalkyl” moieties include, but are not limited to, 1,4-dioxanyl, 1,3-dioxanyl, pyrrolidinyl, pyrrolidin-2-onyl, piperidinyl, imidazolidine-2,4-dionepiperidinyl, piperazinyl, piperazine-2,5-dionyl, morpholinyl, dihydropyranyl, dihydrocinnolinyl, 2,3-dihydrobenzo[1,4] dioxinyl, 3,4-dihydro-2H-benzo[b][1,4]-dioxepinyl, tetrahydropyranyl, 2,3-dihydro furanyl, 2,3-dihydrobenzofuranyl, dihydroisoxazolyl, tetrahydrobenzodiazepinyl, tetrahydroquinolinyl, tetrahydrofuranyl, tetrahydronaphthyridinyl, tetrahydropurinyl, tetrahydrothiopyranyl, tetrahydrothiophenyl, tetrahydroquinoxalinyl, tetrahydropyridinyl, tetrahydrocarbolinyl, 4H-benzo[1,3]-dioxinyl, benzo[1,3]dioxonyl, 2,2-difluorobenzo-[1,3]-dioxonyl, 2,3-dihydro-phthalazine-1,4-dionyl, and isoindole-1,3-dionyl.

As used herein, the term “aryl” refers to an optionally substituted benzene ring or refers to a ring system which may result by fusing one or more optional substituents. Exemplary optional substituents include substituted C₁₋₃ alkyl, substituted C₂₋₃ alkenyl, substituted C₂₋₃ alkynyl, heteroaryl, heterocyclic, aryl, alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen, or ureido. Such a ring or ring system may be optionally fused to aryl rings (including benzene rings) optionally having one or more substituents, carbocycle rings or heterocyclic rings. Examples of “aryl” groups include, but are not limited to, phenyl, naphthyl, tetrahydronaphthyl, biphenyl, indanyl, anthracyl or phenanthryl, as well as substituted derivatives thereof.

As used herein, the term “heteroaryl” refers to an optionally substituted monocyclic five to six-membered aromatic ring containing one or more heteroatomic substitutions selected from S, SO, SO₂, O, N, or N-oxide, or refers to such an aromatic ring fused to one or more rings such as heteroaryl rings, aryl rings, heterocyclic rings, or carbocycle rings (e.g., a bicyclic or tricyclic ring system), each having optional subsituents.

Examples of optional substituents are selected from the group consisting of substituted C₁₋₃ alkyl, substituted C₂₋₃ alkenyl, substituted C₂₋₃ alkynyl, heteroaryl, heterocyclic, aryl, C₁₋₃ alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen or ureido. Examples of “heteroaryl” groups used herein include, but are not limited to, benzoimidazolyl, benzothiazolyl, benzoisothiazolyl, benzothiophenyl, benzopyrazinyl, benzotriazolyl, benzo[1,4] dioxanyl, benzofuranyl, 9H-a-carbolinyl, cinnolinyl, furanyl, furo [2,3-b]pyridinyl, imidazolyl, imidazolidinyl, imidazopyridinyl, isoxazolyl, isothiazolyl, isoquinolinyl, indolyl, indazolyl, indolizinyl, naphthyridinyl, oxazolyl, oxothiadiazolyl, oxadiazolyl, phthalazinyl, pyridyl, pyrrolyl, purinyl, pteridinyl, phenazinyl, pyrazolyl, pyridyl, pyrazolopyrimidinyl, pyrrolizinyl, pyridazyl, pyrazinyl, pyrimidyl, 4-oxo-1,2-dihydro-4H-pyrrolo[3,2,1-ij]-quinolin-4-yl, quinoxalinyl, quinazolinyl, quinolinyl, quinolizinyl, thiophenyl, triazolyl, triazinyl, tetrazolopyrimidinyl, triazolopyrimidinyl, tetrazolyl, thiazolyl, thiazolidinyl, and substituted versions thereof.

As used herein, the term “amino” refers to the group —NH₂. The amino group is optionally substituted with substituted alkyl, substituted carbocycle, aryl, heteroaryl or heterocyclic, as defined above.

As used herein, the term “carbonyl” refers to the group composed of a carbon atom double-bonded to an oxygen atom, ═(C═O).

As used herein, the term “carboxy” refers to the group —C(O)OH. The carboxy group is optionally substituted with substituted alkyl, substituted carbocycle, aryl, heteroaryl or heterocyclic, as defined above.

As used herein, the term “carbamoyl” refers to the group —(C═O)NH₂.

As used herein, the term “cyano” refers to the group —CN.

As used herein, the term “halogen” refers to fluorine (F), chlorine (CO, bromine (Br), or iodine (I).

As used herein, the term “formyl” refers to the group —(C═O)H.

As used herein, the term “hydroxy” refers to the group —OH.

As used herein, the term “mercapto” refers to the group —SH.

As used herein, the term “oxo” refers to the group ═O.

As used herein, the term “alkoxy” refers to the group —ORₐ, where Rₐ is alkyl as defined above. Exemplary alkoxy groups useful in the present invention include, but are not limited to, methoxy, difluoromethoxy, trifluoromethoxy, ethoxy, n-propoxy, isopropoxy, n-butoxy and t-butoxy.

As used herein, the term “alkylcarbonyl” refers to the group —(C═O)Rₐ, wherein Rₐ is alkyl, as defined above. Exemplary alkylcarbonyl groups useful in the present invention include, but are not limited to, methylcarbonyl, ethylcarbonyl, n-propylcarbonyl, isopropylcarbonyl, n-butylcarbonyl group, and iso-butylcarbonyl group.

As used herein, the term “alkoxycarbonyl” refers to the group —(C═O)R_{b}, wherein R_{b} is alkoxy, as defined above. Exemplary alkoxycarbonyl groups useful in the present invention include, but are not limited to, methoxycarbonyl, ethoxycarbonyl, and propoxycarbonyl.

As used herein, the term “sulfanyl” refers to the group —SR^{c}, wherein R_{c} is substituted alkyl, substituted cycloalkyl, aryl, heteroaryl, or heterocycloalkyl, as defined above.

As used herein, the term “sulfonyl” refers to the group —S(O)₂R_{c}, wherein R_{c} is substituted alkyl, substituted cycloalkyl, aryl, heteroaryl, or heterocycloalkyl, as defined above.

As used herein, the term “ureido” refers to the group —NHC(O)NHR_{d} wherein R_{d} is hydrogen, alkyl, alkylthio, cycloalkyl, or aryl as defined above.

It is to be understood that the present invention also includes a pharmaceutically acceptable salt and an acid addition salt of the inventive compound, such as a hydrochloride, trifluoroacetic acid, formic acid, citric acid, fumaric acid, fumarate mono-sodium, p-toluenesulfonic acid, stearic acid, citrate di-sodium, tartaric acid, malic acid, lactic acid, succinic acid, or salicylic acid addition salt. The compounds of the present invention may contain one or more asymmetric carbon atoms and may exist in racemic and optically active forms. All of these compounds and diastereomers are incorporated within the scope of the present invention.

In accordance with one aspect of the present invention, there are provided a compound of formula (I) or a pharmaceutically acceptable salt, or a prodrug thereof, and a method for preparing the same:

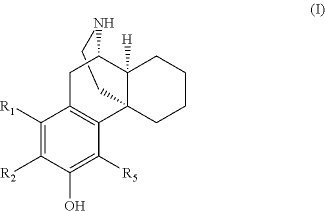

wherein,

R₁ is selected from the group consisting of hydrogen, C₁-C₃ alkyl, C₃-C₅ cycloalkyl, and halogen;

R₂ is selected from the group consisting of hydrogen; hydroxyl; mercapto; sulfanyl; sulfonyl; formyl; carboxyl; —NR₃R₄; halogen; C₁-C₁₀ alkyl; C₁-C₁₀ alkoxy; C₃-C₇ cycloalkyl; heterocycloalkyl; aryl; heteroaryl; —C₁-C₄ alkyl-Ar; and C₁-C₁₀ alkyl, C₃-C₇ cycloalkyl, heterocycloalkyl, aryl, heteroaryl, and —C₁-C₄ alkyl-Ar substituted with one or more Z groups, Ar being selected from the group consisting of phenyl, naphthyl, furyl, pyridyl, thienyl, thiazolyl, isothiazolyl, pyrazolyl, triazolyl, tetrazolyl, imidazolyl, imidazolidinyl, benzofuranyl, indolyl, thiazolidinyl, isoxazolyl, oxadiazolyl, thiadiazolyl, morpholinyl, piperidinyl, piperazinyl, pyrrolyl, and pyrimidinyl, and Z being independently selected from the group consisting of hydroxyl, C₁-C₄ alkyl, C₁-C₄ alkoxy, —(CH₂)ₘC(O)OR₃, —C(O)NR₃R₄, —CN, —(CH₂)ₙOH, —NO₂, F, Cl, Br, I, —NR₃R₄ and —NHC(O)R₃, wherein m is 0 to 4 and n is 0 to 4;

R₃ and R₄ are independently selected from the group consisting of hydrogen; C₁-C₆ alkyl; C₁-C₆ alkyl substituted with one or two R₇ groups; C₁-C₆ alkoxy; C₃-C₆ cycloalkyl; heterocycloalkyl; phenyl; heteroaryl; and C₃-C₆ cycloalkyl, heterocycloalkyl, phenyl, and heteroaryl substituted with one to three R₆ groups; or R₃ and R₄ are joined together with the N-atom to which they are attached, forming a heterocycloalkyl group or a heterocycloalkyl group substituted with one to three R₆ groups;

each R₆ is independently selected from the group consisting of hydroxyl; C₁-C₆ alkyl; C₁-C₆ alkyl substituted with one to three R₇ groups; C₁-C₆ alkoxy; halo(C₁-C₆ alkoxy); C₃-C₆ cycloalkyl; C₃-C₆ cycloalkyl substituted with one —NRₐR_{b} or pyrrolidinyl; heterocycloalkyl; phenyl; heteroaryl; —C(O)NRₐR_{b}; —C(O)R_{c}; —C(O)OR_{c}; oxo; cyano; —NRₐR_{b}; halogen; (C₁-C₆ alkyl)ureido, arylureido, and (C₁-C₆ alkylthio)ureido;

each R₇ is independently selected from the group consisting of hydroxyl; C₁-C₃ alkoxy; halogen; phenyl; cyano; —NRₐR_{b}; —C(O)NRₐR_{b}; —C(O)R_{c}; C₃-C₆ cycloalkyl; C₃-C₆ cycloalkyl substituted with one hydroxyl, heterocycloalkyl or —NRₐR_{b} group; heterocycloalkyl; heteroaryl; and heteroaryl substituted with one methyl, —NRₐR_{b} or hydroxyl;

each Rₐ is independently selected from the group consisting of hydrogen; C₁-C₃ alkyl; and C₁-C₃ alkyl substituted with one hydroxyl, methoxy, or dimethylamine; (C₁-C₄ alkyl)sulfonyl; arylsulfonyl; (C₁-C₄ alkyl)carbonyl; and (C₁-C₄ alkoxy)carbonyl,

each R_{b} is independently selected from the group consisting of hydrogen and C₁-C₃ alkyl;

each R_{c} is independently selected from the group consisting of hydrogen; C₁-C₃ alkyl; C₁-C₃ alkyl substituted with one methoxy group; phenyl; heterocycloalkyl; and heteroaryl;

R₅ is selected from the group consisting of hydrogen and halogen, or R₅ is joined together with adjacent hydroxyl group to form a heterocycloaryl group having two oxygens.

In the compounds of formula (I), preferably, each R₆ is independently selected from the group consisting of C₁-C₄ alkyl, C₁-C₄ haloalkyl, halogen, hydroxyl, methoxy, ethoxy, C₁-C₃ haloalkoxy, amino, phenyl, benzyl, carbamoyl, cyano, methoxycarbonyl, ethoxycarbonyl, carboxyl, (C₁-C₄ alkyl)sulfonamido, benzenesulfonamido, pivalamido, acetamido, ethylureido, phenylureido, butylureido, and butylthioureido; and

R₇ is C₁-C₃ alkoxy or fluoro.

Preferable compounds of the present invention are those of formula (I) wherein, R₁ is selected from the group consisting of hydrogen, methyl, cylcopropyl, chloro, and bromo;

R₂ is selected from the group consisting of hydroxyl, hydrogen, C₁-C₄ alkyl, C₁-C₄ haloalkyl, C₁-C₄ hydroxyalkyl, C₁-C₄ alkoxy, C₁-C₄ alkylthiol, phenylthiol, formyl, carboxyl, fluoro, chloro, bromo, iodo, (C₁-C₄ alkyl)C₃-C₇ cycloalkyl, —NR₃R₄, cyanophenyl, halophenyl, azepanyl, piperidinyl, (C₁-C₄ alkyl)piperidinyl, pyrrolidinyl, (C₁-C₄ alkyl)piperazinyl, and morpholino; and

R₃ and R₄ are independently selected from the group consisting of hydrogen, C₁-C₄ alkyl, phenyl, pyridinyl, benzodioxol, dihydrobenzo[1,4]dioxin, quinolinyl, isoquinolinyl, 1H-indazol-5-yl, 5,6,7,8-tetrahydronaphthalen-2-yl, cyclopentyl, cyclohexyl, fluorophenyl, and piperidin-1-yl.

Compounds especially useful in the present invention are selected from the group consisting of:
(+)-2-Fluoro-3-hydroxymorphinan TFA salt;
(+)-4-Chloro-2-fluoro-3-hydroxymorphinan TFA salt;
(+)-4-Bromo-2-fluoro-3-hydroxymorphinan TFA salt;
(+)-2,4-Dichloro-3-hydroxymorphinan TFA salt;
(+)-4-Chloro-3-hydroxymorphinan TFA salt;
(+)-2,4-Dibromo-3-hydroxymorphinan TFA salt;
(+)-4-Bromo-3-hydroxymorphinan TFA salt;
(+)-4-Bromo-2-chloro-3-hydroxymorphinan TFA salt;
(+)-3-Hydroxy-2-iodomorphinan TFA salt;
(+)-2,3-Dihydroxymorphinan TFA salt;
(+)-3,4-(Methylenedioxy)morphinan TFA salt;
(+)-3-Hydroxy-2-methoxymorphinan TFA salt;
(+)-2-Formyl-3-hydroxymorphinan TFA salt;
((+)-3-Hydroxymorphinan)-2-carboxylic acid TFA salt;
(+)-2-(Difluoromethyl)-3-hydroxymorphinan TFA salt;
(+)-3-Hydroxy-2-(hydroxymethyl)morphinan TFA salt;
(+)-2-(Azepan-1-yl)-3-hydroxymorphinan TFA salt;
(+)-3-Hydroxy-2-(methylamino)morphinan TFA salt;
(+)-3-Hydroxy-2-(4-methylpiperidin-1-yl)morphinan TFA salt;
(+)-2-(tert-Butylamino)-3-hydroxymorphinan TFA salt;
(+)-3-Hydroxy-2-(piperidin-1-yl)morphinan TFA salt;
(+)-3-Hydroxy-2-(pyrrolidin-1-yl)morphinan TFA salt;
(+)-2-Ethylamino-3-hydroxymorphinan TFA salt;
(+)-3-Hydroxy-2-(4-methylpiperazin-1-yl)morphinan 2TFA salt;
(+)-2-(4-Chlorophenylamino)-3-hydroxymorphinan TFA salt;
(+)-3-Hydroxy-2-(4-hydroxyphenylamino)morphinan TFA salt;
(+)-2-(3,5-Dimethylphenylamino)-3-hydroxymorphinan TFA salt;
(+)-3-Hydroxy-2-(4-methylphenylamino)morphinan TFA salt;
(+)-2-(4-Fluorophenylamino)-3-hydroxymorphinan TFA salt;
(+)-3-Hydroxy-2-(phenylamino)morphinan TFA salt;
(+)-3-Hydroxy-2-(4-methoxyphenylamino)morphinan TFA salt;
(+)-2-(4-Aminophenyamino)-3-hydroxymorphinan TFA salt;
(+)-2-(4-Bromophenylamino)-3-hydroxymorphinan TFA salt;
(+)-3-Hydroxy-2-(pyridin-2-ylamino)morphinan 2TFA salt;
(+)-3-Hydroxy-2-(4-(trifluoromethyl)phenylamino)morphinan TFA salt;
(+)-3-Hydroxy-2-((3,4-methylendioxy)phenylamino)morphinan TFA salt;
(+)-2-((3,4-Ethylenedioxy)phenylamino)-3-hydroxymorphinan TFA salt;
(+)-2-(2-Fluorophenylamino)-3-hydroxymorphinan TFA salt;
(+)-2-(3-Fluorophenylamino)-3-hydroxymorphinan TFA salt;
(+)-2-(2,4-Dimethoxyphenylamino)-3-hydroxymorphinan TFA salt;
(+)-3-Hydroxy-2-(2-methylphenylamino)morphinan TFA salt;
(+)-3-Hydroxy-2-(2-methoxyphenylamino)morphinan TFA salt;
(+)-3-Hydroxy-2-(2-hydroxyphenylamino)morphinan TFA salt;
(+)-3-Hydroxy-2-(3-hydroxyphenylamino)morphinan TFA salt;
(+)-2-(2,4-Dihydroxyphenylamino)-3-hydroxymorphinan TFA salt;
(+)-2-(4-Hydroxyphenylamino)-3-hydroxymorphinan TFA salt;
(+)-2-(2,6-Dihydroxyphenylamino)-3-hydroxymorphinan TFA salt;
(+)-2-(2-Chlorophenylamino)-3-hydroxymorphinan TFA salt;
(+)-2-(2-Ethylphenylamino)-3-hydroxymorphinan TFA salt;
(+)-3-Hydroxy-2-(2-isopropylphenylamino)morphinan TFA salt;
(+)-2-(2-t-Butylphenylamino)-3-hydroxymorphinan TFA salt;
(+)-3-Hydroxy-2-(2-(trifluoromethyl)phenylamino)morphinan TFA salt;
(+)-2-(4-Ethylphenylamino)-3-hydroxymorphinan TFA salt;
(+)-3-Hydroxy-2-(4-isopropylphenylamino)morphinan TFA salt;
(+)-2-(3-Chloro-2-hydroxyphenylamino)-3-hydroxymorphinan TFA salt;
(+)-2-(5-Fluoro-2-hydroxyphenylamino)-3-hydroxymorphinan TFA salt;
(+)-2-(3-Fluoro-2-hydroxyphenylamino)-3-hydroxymorphinan TFA salt;
(+)-3-Hydroxy-2-(4-(trifluoromethoxy)phenylamino)morphinan TFA salt;
(+)-3-Hydroxy-2-(2-(trifluoromethoxy)phenylamino)morphinan TFA salt;
(+)-2-(Biphenyl-2-ylamino)-3-hydroxymorphinan TFA salt;
(+)-2-(2-Carbamoylphenylamino)-3-hydroxymorphinan TFA salt;
(+)-2-(2-Benzylphenylamino)-3-hydroxymorphinan TFA salt;
(+)-2-(3,4-Dimethoxyphenylamino)-3-hydroxymorphinan TFA salt;
(+)-2-(2,5-Dichlorophenylamino)-3-hydroxymorphinan TFA salt;
(+)-2-(3,4-Dichlorophenylamino)-3-hydroxymorphinan TFA salt;
(+)-3-Hydroxy-2-(quinolin-8-ylamino)morphinan TFA salt;
(+)-3-Hydroxy-2-(isoquinolin-5-ylamino)morphinan TFA salt;
(+)-3-Hydroxy-2-(quinolin-6-ylamino)morphinan TFA salt;
(+)-3-Hydroxy-2-((1H-indazol-5-yl)amino)morphinan TFA salt;
(+)-3-Hydroxy-2-((1H-indazol-5-yl)amino)morphinan TFA salt;
(+)-3-Hydroxy-2-((5,6,7,8-tetrahydronaphthalen-2-yl)amino)morphinan TFA salt;
(+)-3-Hydroxy-2-methylthiomorphinan TFA salt;
(+)-3-Hydroxy-2-phenylthiomorphinan TFA salt;
(+)-2-(4-Chlorophenyl)-3-hydroxymorphinan TFA salt;
(+)-3-Hydroxy-2-(4-methylphenyl)morphinan TFA salt;
(+)-2-(2,4-Dichlorophenyl)-3-hydroxymorphinan TFA salt;
(+)-2-(4-Fluorophenyl)-3-hydroxymorphinan TFA salt;
(+)-2-(3-Cyanophenyl)-3-hydroxymorphinan TFA salt;
(+)-3-Hydroxy-2-(4-trifluorophenyl)morphinan TFA salt;
(+)-3-Hydroxy-2-phenylmorphinan TFA salt;
(+)-3-Hydroxy-2-isobutylmorphinan TFA salt;
(+)-3-Hydroxy-2-propylmorphinan TFA salt;
(+)-2-Butyl-3-hydroxymorphinan TFA salt;
(+)-2-Ethyl-3-hydroxymorphinan TFA salt;
(+)-3-Hydroxy-2-methylmorphinan TFA salt;
(+)-3-Hydroxy-2-morpholinomorphinan TFA salt;
(+)-3-Hydroxy-2-isopropylaminomorphinan TFA salt;
(+)-3-Hydroxy-2-propylaminomorphinan TFA salt;
(+)-2-(Heptan-4-ylamino)-3-hydroxymorphinan TFA salt;
(+)-2-Butylamino-3-hydroxymorphinan TFA salt;
(+)-3-Hydroxy-2-(1-phenylethylamino)morphinan TFA salt;
(+)-2-Cyclopentylamino-3-hydroxymorphinan TFA salt;
(+)-2-Cyclohexylamino-3-hydroxymorphinan TFA salt;
(+)-2-Cycloheptylamino-3-hydroxymorphinan TFA salt;
(+)-2-(sec-Butylamino)-3-hydroxymorphinan TFA salt;
(+)-2-(Dipropylamino)-3-hydroxymorphinan TFA salt;
(+)-3-Hydroxy-2-(3-trifluoropropylamino)morphinan TFA salt;
(+)-2-(Dimethylamino)-3-hydroxymorphinan TFA salt;
(+)-2-(2-Ethoxyethylamino)-3-hydroxymorphinan TFA salt;
(+)-3-Hydroxy-2-(2-hydroxyethylamino)morphinan TFA salt;
(+)-2-(Diethylamino)-3-hydroxymorphinan TFA salt;
(+)-3-Hydroxy-2-(methylpropylamino)morphinan TFA salt;
(+)-2-(Ethylmethylamino)-3-hydroxymorphinan TFA salt;
(+)-2-tert-Butyl-3-hydroxymorphinan TFA salt;
(+)-3-Hydroxy-2-(1-methylcyclohexyl)morphinan TFA salt;
(+)-3-Hydroxy-2-morpholinomorphinan TFA salt;
(+)-2-(2-Cyanophenylamino)-3-hydroxymorphinan TFA salt;
(+)-3-Hydroxy-2-(2-(methoxycarbonyl)phenylamino)morphinan TFA salt;
(+)-2-(2-Carboxyphenylamino)-3-hydroxymorphinan TFA salt;
(+)-3-Hydroxy-2-(2-(methanesulfonamido)phenylamino)morphinan TFA salt;
(+)-2-(2-(Ethanesulfonamido)phenylamino)-3-hydroxymorphinan TFA salt;
(+)-2-(2-(Butanesulfonamido)phenylamino)-3-hydroxymorphinan TFA salt;
(+)-2-(2-(Benzenesulfonamido)phenylamino)-3-hydroxymorphinan TFA salt;
(+)-3-Hydroxy-2-(4-(methanesulfonamido)phenylamino)morphinan TFA salt;
(+)-3-Hydroxy-2-(2-(pivalamido)phenylamino)morphinan TFA salt;
(+)-2-(2-(Acetamido)phenylamino)-3-hydroxymorphinan TFA salt;
(+)-2-(2-(Ethoxycarbonylamino)phenylamino)-3-hydroxymorphinan TFA salt;
(+)-2-(2-(Butoxycarbonylamino)phenylamino)-3-hydroxymorphinan TFA salt;
(+)-3-Hydroxy-2-(2-(isobutyloxycarbonylamino)phenylamino)morphinan TFA salt;
(+)-2-(2-(Ethylureido)phenylamino)-3-hydroxymorphinan TFA salt;
(+)-2-(2-(Butylureido)phenylamino)-3-hydroxymorphinan TFA salt;
(+)-3-Hydroxy-2-(2-(phenylureido)phenylamino)morphinan TFA salt;
(+)-2-(2-(Butylthioureido)phenylamino)-3-hydroxymorphinan TFA salt;
(+)-2-(4-Fluorophenyl(methyl)amino)-3-hydroxymorphinan TFA salt;
(+)-3-Hydroxy-2-(2-(methanesulfonamido)phenyl(methyl)amino) morphinan TFA salt;
(+)-2-(2-(Dimethylamino)phenylamino)-3-hydroxymorphinan TFA salt;
(+)-3-Hydroxy-2-(2-(methylamino)phenylamino)morphinan TFA salt;
(+)-3-Hydroxy-2-(2-(piperidin-1-yl)phenylamino)morphinan TFA salt;
(+)-2-(2-(Diethylamino)phenylamino)-3-hydroxymorphinan TFA salt;
(+)-2-(2-(Ethylamino)phenylamino)-3-hydroxymorphinan TFA salt;
(+)-2-(2-(Ethyl(methyl)amino)phenylamino)-3-hydroxymorphinan TFA salt;
(+)-1-Bromo-3-hydroxy-2-(2-(methanesulfonamido)phenylamino) morphinan TFA salt;
(+)-1-Chloro-3-hydroxy-2-(2-(methanesulfonamido)phenylamino) morphinan TFA salt;
(+)-3-Hydroxy-2-(2-(methanesulfonamido)phenylamino)-1-methylmorphinan TFA salt; and
(+)-1-Cylcopropyl-3-hydroxy-2-(2-(methanesulfonamido)phenylamino) morphinan TFA salt.

### General Synthesis of the Compounds

The compounds of the present invention and the preparation thereof will be better understood in connection with the following reaction schemes, which are merely illustrative of the methods by which the compounds of the invention may be prepared and are not intended to limit the scope of the invention as defined in the appended claims.

As shown in the following Reaction Scheme 1, the compound of formula 1 (Cbz-HM 1) is prepared by conducing an amino protecting reaction of (+)-3-hydroxymorphinan HBr (3-HM.HBr) using benzyloxycarbonyl chloride (Cbz-Cl) and converted to 2-fluoro analogue 2 by conducting an electrophilic fluorination with 1-fluoropyridinium triflate (NFPT) at heating conditions. The hydrogenation of the Cbz-protective group of 2-fluoro analogue 2 using a palladium (10% Pd/C) catalyst is performed in alcoholic solvent to yield the Cbz-deprotected analogue 3. Chlorination of this monofluoride compound 3 with sulfuryl chloride in acetic acid gives 4-chloro analogue 4, whereas bromination with bromine in acetic acid gives 4-bromo analogue 5.

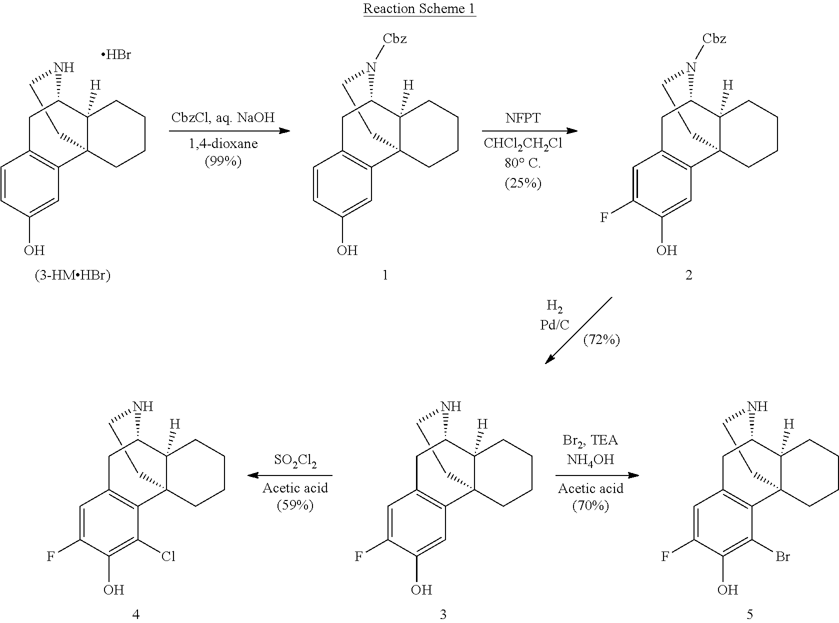

As shown in the following Reaction Scheme 2,3-HM.HBr is neutralized to remove bromide salt which interrupts the chlorination. The resulting HM free amine form (3-HM) is chlorinated at both 2- and 4-positions using sulfuryl chloride, respectively. Optionally, the resulting compound of formula 6 is subjected to hydrogenation in the presence of palladium catalyst to obtain the compound of formula 7.

Alternatively, 3-HM.HBr is brominated at 2- and 4-positions using bromine. Optionally, the resulting compound of formula 8 is subjected to a hydrogenation in the presence of palladium catalyst to obtain the compound of formula 9, which is then chlorinated at 2-position using sulfuryl chloride to provide corresponding compound of formula 10.

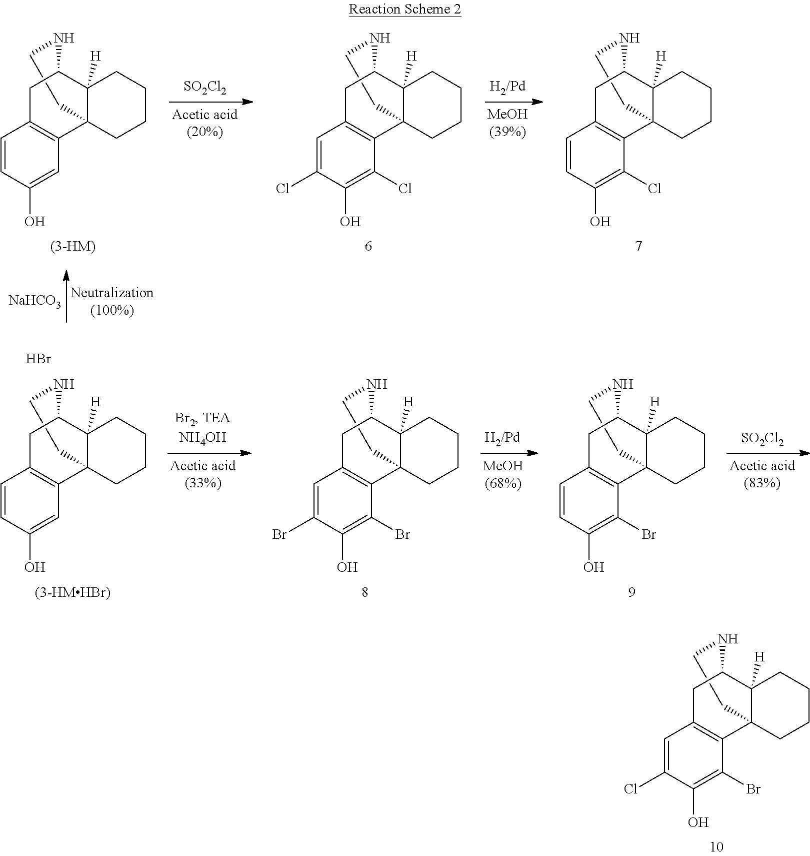

As shown in the following Scheme 3, the 2-iodo analogue 12 of the (+)-3-Hydroxy-N-(tert-butyloxycarbonyl)morphinan (N-Boc-protected HM) 11 is obtained using N-iodosuccinimide (NIS) as an electrophilic iodinating agent. The N-Boc protective group can be deprotected efficiently with 4M HCl in dioxane to afford the mono-iodide derivative 13.

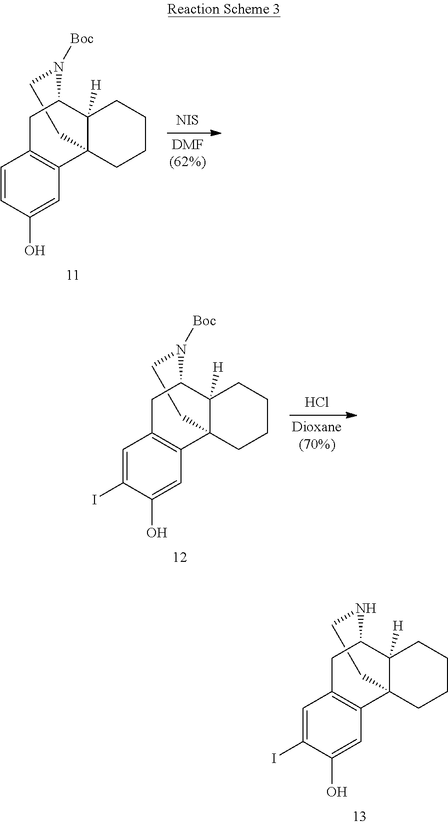

As shown in the following Reaction Scheme 4, the treatment of Cbz-HM 1 with 1-hydroxy-1,2-benziodoxol-3(1H)-one 1-oxide (IBX) produces both 2,3- and 3,4-quinone intermediates, which are subsequently followed by the reduction with cold methanolic NaBH₄ to provide corresponding o-diphenol derivatives (14, 16). A 3,4-diol compound 16 can be converted to the corresponding benzodioxole derivative 17 by the reaction with diiodomethane in the presence of K₂CO₃ as a base. The 2-iodo analogue 19 of Cbz-HM 1 is prepared using N-iodosuccinimide (NIS) as an electrophilic iodinating agent. Treatment of 2-iodo analogue 19 with NaOMe in the presence of CuCl₂ gives the corresponding methoxy derivative 20. The Cbz-deprotection of the compound of formula 14, 17 or 20 can be efficiently achieved by the general hydrogenation procedure in the presence of palladium (10% Pd/C) catalyst in alcoholic solvent to provide the corresponding Cbz-deprotected analogues (15, 18, 21).

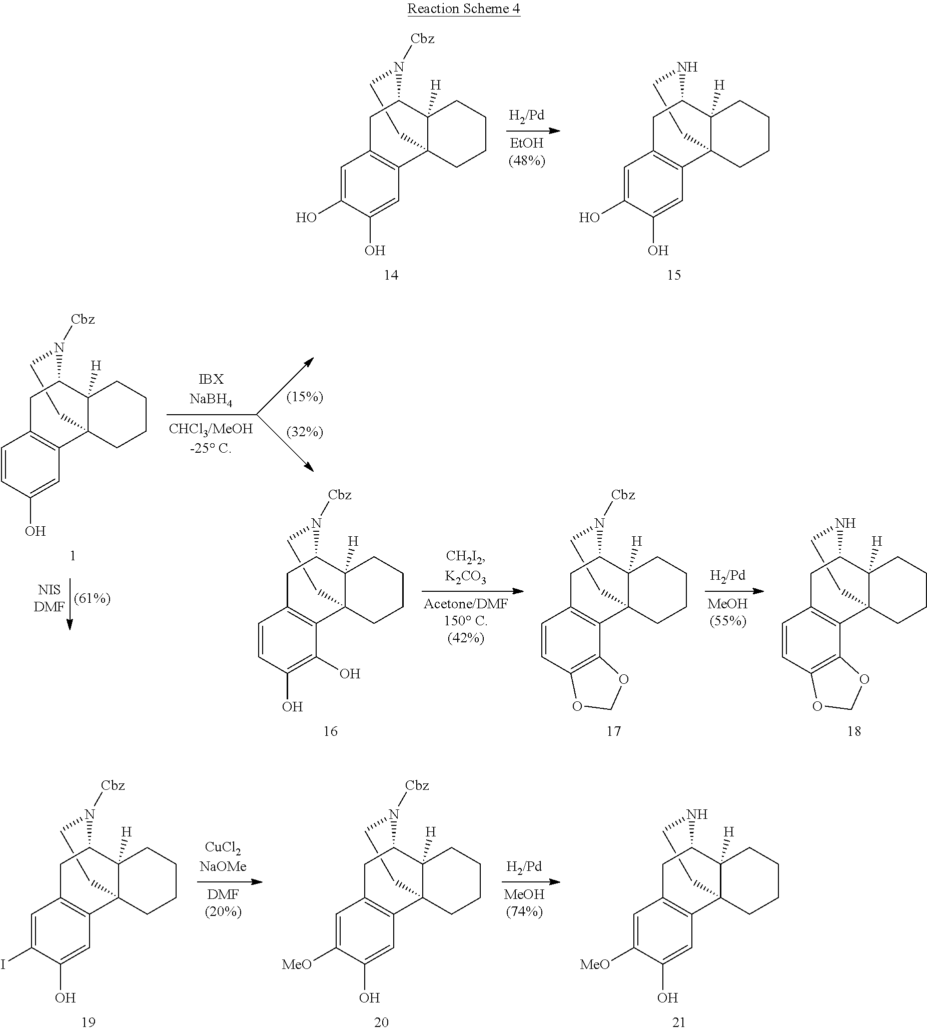

A selective ortho-formylation of Cbz-HM 1 that involves heating the mixture of Cbz-HM 1, anhydrous MgCl₂, triethylamine (TEA), and paraformaldehyde under reflux in acetonitrile (ACN) is shown in the following Reaction Scheme 5 [Hansen, T. V. et al. Tetrahedron Lett. 2005, 46, 3357-3358]. Hydrogenation of the resulting benzaldehyde 22 on Pd/C, followed by purification by prep HPLC in the presence of a small amount of trifluoroacetic acid (TFA) provides the formyl derivative A.

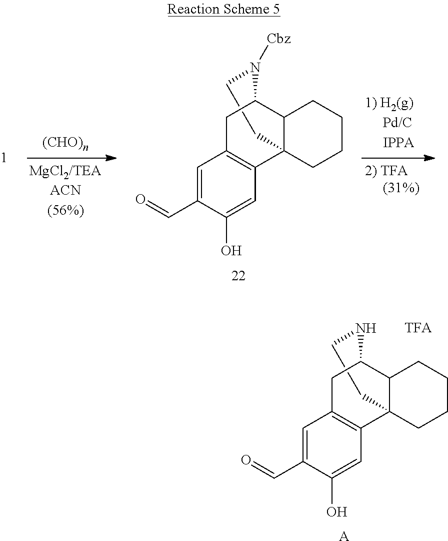

As shown in the following Reaction Scheme 6, benzaldehyde 22 can be oxidized to the corresponding carboxylic acid 23 using a suitable oxidizing agent, such as KMnO₄. Geminal difluoride analogue 25 from benzaldehyde 22 is effectively prepared using diethylaminosulfur trifluoride (DAST) as a fluorinating agent in dichlromethane (DCM). Reduction of the benzaldehyde 22 can be achieved using reducing agent such as alcoholic NaBH₄ to gives the corresponding benzyl alcohol 27. Final deprotection of the compound of formula 23, 25 and 27 by hydrogenation using palladium catalyst (e.g., 10% Pd/C) provides the desired compounds (24, 26, 28) with good yields.

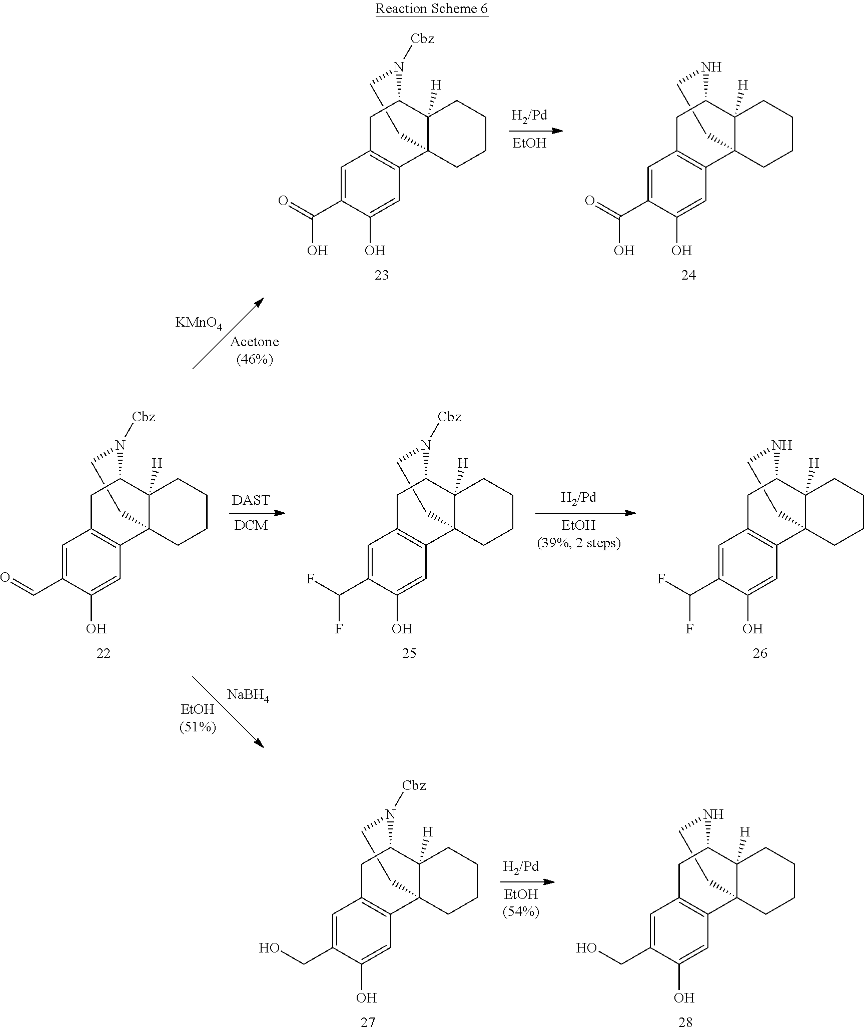

As shown in the following Reaction Scheme 7,3-HM.HBr undergoes iodination under conditions of the mixture of iodine and potassium iodide in aqueous sodium hydroxide [Danso-Danquah, R. et al. J. Med. Chem. 1995, 38, 2986-2989] to give iodide 13. Protection of the iodide 13 with Cbz-Cl, and subsequent methylation produce a key intermediate 20 in approximately 60% yield.

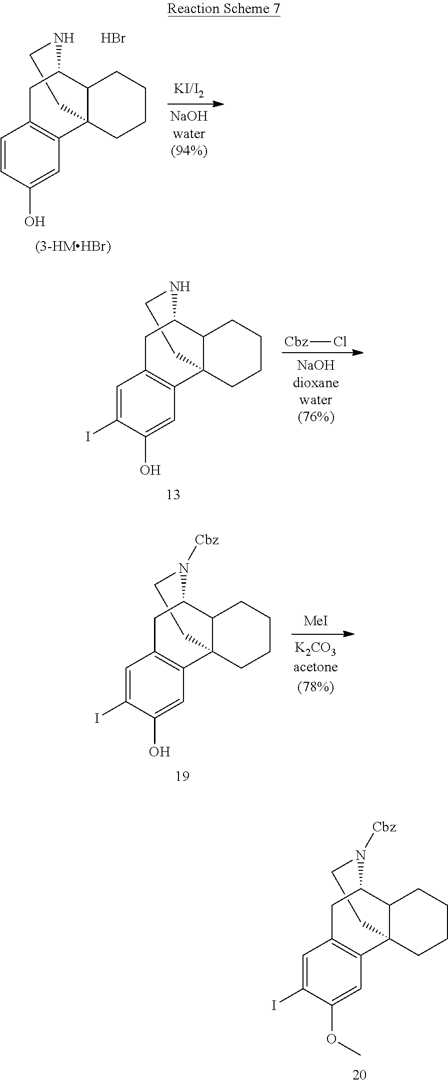

As shown in the following Reaction Scheme 8, the key intermediate 20 prepared above is coupled with cyclic amine in the presence of Pd₂(dba)₃, racemic 2,2′-bis(diphenylphosphino)-1,1′-binaphthyl (BINAP), NaOt-Bu and 15-crown-5 in a suitable solvent such as THF [Miguel, G. B. et al. WO 2005/030188 (2005)]. The resulting coupled product is treated with BBr₃ to generate a compound of formula 29 after purification by prep HPLC in the presence of a small amount of TFA in moderate yields. Likewise, aniline or alkylamine is coupled with the key intermediate 20 in the presence of (dppf)PdCl₂.CH₂Cl₂, dppf, and NaOt-Bu in a suitable solvent such as THF [Hartwig, J. F. et al. J. Am. Chem. Soc. 1996, 118, 7217-7218]. The resulting coupled product is treated using the same method as described above to provide a compound of formula 30.

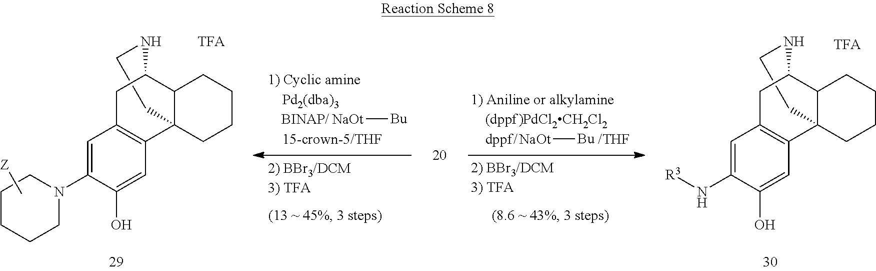

wherein, Z and R₃ are the same as defined above.

As shown in the following Reaction Scheme 9, S-linked compounds are prepared in an analogous fashion. Thus, treatment of the key intermediate 20 with various thiols in the presence of a catalyst (e.g., Pd(PPh₃)₄) and NaOt-Bu in a suitable solvent such as EtOH, and subsequent demethylation using BBr₃ provides a compound of formula 31 after purification by prep HPLC in the presence of a small amount of TFA in reasonable yields. On the other hand, palladium-catalyzed Suzuki-Miyaura cross-coupling reaction of the key intermediate 20 with arylboronic acid and followed by demethylation using BBr₃ smoothly affords the diaryl compounds 32. Alkyl groups in lieu of aryl groups can be installed as well by use of Suzuki-Miyaura coupling reaction to give a compound of formula 33 after routine demethylation process as shown in the following Reaction Scheme 10.

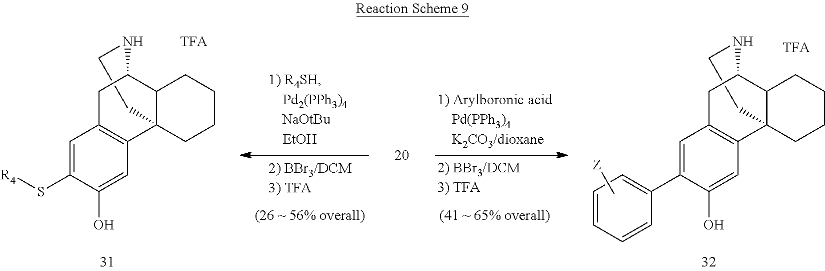

wherein, Z and R₄ are the same as defined above.

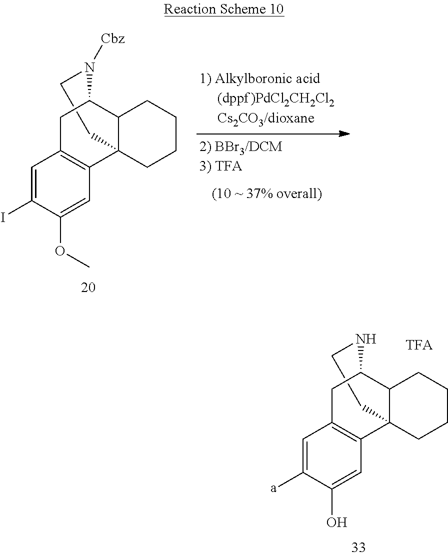

wherein, a is alkyl.

Another way of preparing amino-morphinans is described in the following Reaction Scheme 11. Thus, neutralized 3-HM is treated with HNO₃/formic acid to give 2-nitro-3-hydroxymorphinan, which is converted to the compound of formula 34 after amino protection by employing a protective group such as cbz [Peng, X. et al. Bioorg. Med. Chem. 2007, 15, 4106-4112]. Methylation of the compound of formula 34, followed by selective reduction using a reducing agent such as hydrazine in the presence of a catalyst Raney Ni in a suitable solvent such as MeOH, generate the critical intermediate, aniline 36 in approximately 80% yield [Yuste, F. et al. Tetrahedron Lett. 1982, 23, 147-148].

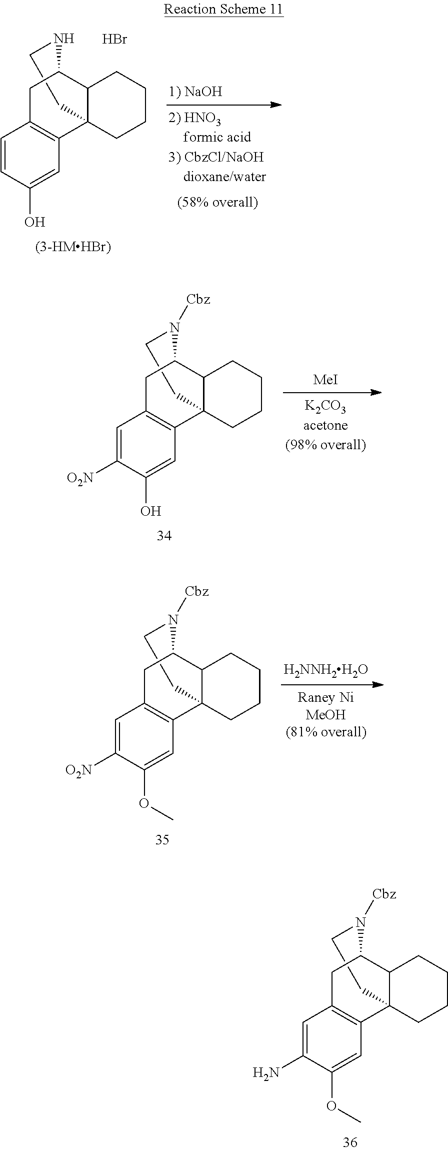

As demonstrated in the following Reaction Scheme 12, aniline 36 is treated with 2-chloroethyl ether in the presence of a base such as sodium bicarbonate in a suitable solvent such as DMF to obtain the resulting compound in 90% yield. Then demethylation the resulting compound using BBr₃ in methylene chloride provides the compound of formula 37. On the other hand, reductive alkylation of aniline 36, followed by demethylation using BBr₃, provides a compound of formula 38 in good yields.

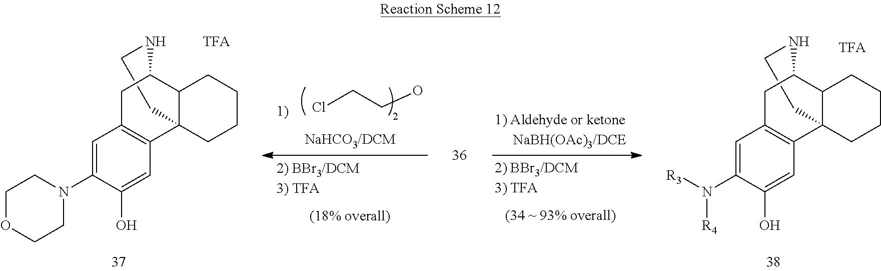

wherein, R₃ and R₄ are the same as defined above.

Another way of preparing aniline derivatives is shown in the following Reaction Scheme 13. Thus, protection of aniline 36 with di-tert-butyl-dicarbonate ((BOC)₂O) generates the compound of formula 39, which is alkylated to provide the compound of formula 40. After deprotection of BOC group of the compound of formula 40 using TFA, the corresponding aniline derivative is utilized for reductive alkylation, leading to the target aniline analogue 41.

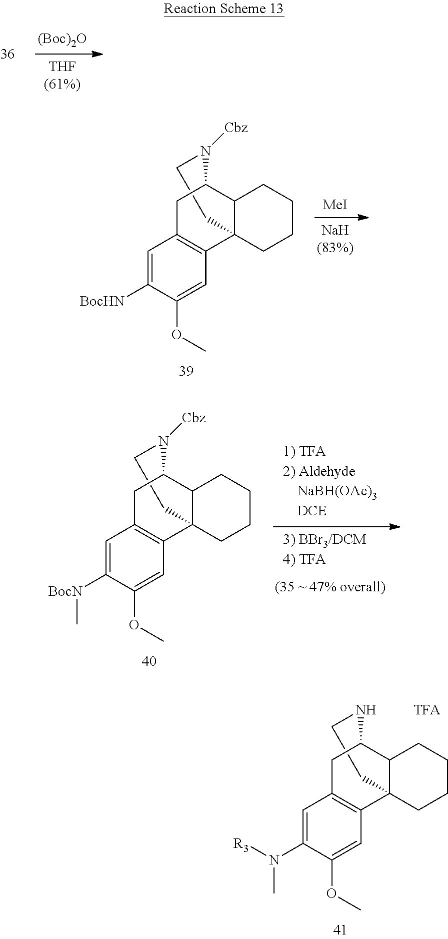

wherein, R₃ is the same as defined above.

As shown in the following Reaction Scheme 14, 2-t-butyl type compound 42 is obtained by treating 3-HM.HBr with tertiary alcohol such as t-butanol in the presence of acid such as conc. sulfuric acid, followed by the purification by prep HPLC in the presence of a small amount of TFA [Jean-Michel, B. et al. U.S. Pat. No. 5,387,594 (1995)].

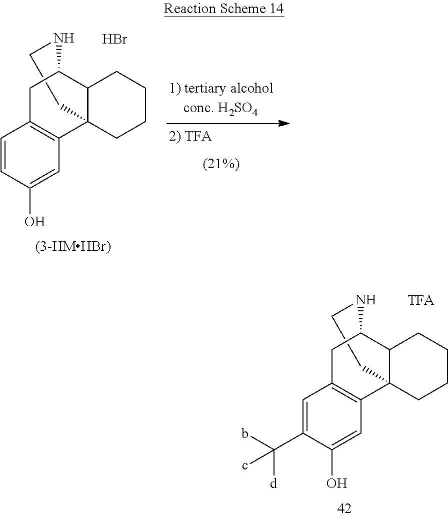

wherein, b, c and d are each independently C₁-C₆ alkyl.

The compounds of the invention may exist in a solid or liquid form. In the solid state, the compounds of the invention may exist in crystalline or noncrystalline form, or as a mixture thereof. For compounds of the invention that are in crystalline form, the skilled artisan will appreciate that pharmaceutically-acceptable solvates may be formed wherein solvent molecules are incorporated into the crystalline lattice during crystallization. Solvates may involve nonaqueous solvents such as acetone, ethanol, n-propanol, isopropanol, n-butanol, tert-butanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The skilled artisan will further appreciate that certain compounds of the invention that exist in crystalline form, including the various solvates thereof, may exhibit polymorphism (i.e., the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The compound of formula (I) is subjected to the hydrolysis in vivo and, then, converted into its parent compound, i.e., (+)-3-HM which is effective as a neuroprotective agent for a neurodegenerative disease. Accordingly, the compound of formula (I) is useful in treating or preventing the neurodegenerative disease including Alzheimer's disease (AD), Parkinson's disease (PD), and Huntington's disease (HD).

In accordance with another aspect of the present invention, there is provided a method for preventing or treating the neurodegenerative disease, which comprises administering the compound of formula (I), or the pharmaceutically acceptable salt or a prodrug thereof to a mammal in need thereof.

In accordance with further aspect of the present invention, there is provided a pharmaceutical composition for preventing or treating the neurodegenerative disease, which comprises the compound of formula (I) or the pharmaceutically acceptable salt or a prodrug thereof as an active ingredient, and a pharmaceutically acceptable carrier.

The pharmaceutical composition may be administered orally, intramuscularly or subcutaneously. The formulation for oral administration may take various forms such as a syrup, tablet, capsule, cream and lozenge. A syrup formulation will generally contain a suspension or solution of the compound or its salt in a liquid carrier, e.g., ethanol, peanut oil, olive oil, glycerine or water, optionally with a flavoring or coloring agent. When the composition is in the form of a tablet, any one of pharmaceutical carriers routinely used for preparing solid formulations may be used. Examples of such carriers include magnesium stearate, terra alba, talc, gelatin, acacia, stearic acid, starch, lactose and sucrose. When the composition is in the form of a capsule, any of the routine encapsulation procedures may be employed, e.g., using the aforementioned carriers in a hard gelatin capsule shell. When the composition is formulated in the form of a soft gelatin shell capsule, any of the pharmaceutical carriers routinely used for preparing dispersions or suspensions may be prepared using an aqueous gum, cellulose, silicate or oil. The formulation for intramuscular or subcutaneous administration may take a liquid form such as a solution, suspension and emulsion which includes aqueous solvents such as water, physiological saline and Ringer's solution; or lipophilic solvents such as fatty oil, sesame oil, corn oil and synthetic fatty acid ester.

Preferably the composition is formulated in a specific dosage form for a particular patient.

Each dosage unit for oral administration contains suitably from 0.1 mg to 500 mg/kg, and preferably from 1 mg to 100 mg/kg of the compound of Formula (I) or its pharmaceutically acceptable salt.

The suitable daily dosage for oral administration is about 0.1 mg/kg to 3 g/kg of the compound of Formula (I) or its pharmaceutically acceptable salt, and may be administered 1 to 3 times a day or every two days, depending on the patient's condition.

The present invention is further described and illustrated in Examples provided below, which are, however, not intended to limit the scope of the present invention.

### EXAMPLE

Unless otherwise noted, all starting materials were obtained from commercial suppliers and used without further purification.

As used herein, the symbols and conventions used describing the processes, schemes and examples of the present invention are consistent with those used in the contemporary scientific literature, for example, the Journal of the American Chemical Society or the Journal of Biological Chemistry. The following abbreviations are used in the Examples:
Hz (Hertz)
TLC (thin layer chromatography)
Tᵣ (retention time)
RP (reverse phase)
MeOH (methanol)
i-PrOH (isopropanol)
TFA (trifluoroacetic acid)
TEA (triethylamine)
EtOH (ethanol)
THF (tetrahydrofuran)
DMSO (dimethylsulfoxide)
EtOAc (ethyl acetate)
DCM (dichlromethane)
HOAc (acetic acid)
DMF (N,N-dimethylformamide)
Ac (acetyl)
CDI (1,1-carbnyldiimidazole)
Bn (benzyl)
HOSu (N-hydroxysuccinimide)
HOBT (1-hydroxybenzotriazole)
Boc (tert-butyloxycarbonyl)
mCPBA (meta-chloroperbenzoic acid)
FMOC (9-fluorenylmethoxycarbonyl)
DCC (dicyclohexylcarbodiimide)
Cbz (benzyloxycarbonyl)
NMM (N-methyl morpholine)
HOAt (1-hydroxy-7-azabenzotriazole)
TBAF (tetra-n-butylammonium fluoride)
THP (tetrahydro-2H-pyran-2-yl)
DMAP (4-dimethylaminopyridine)
HPLC (high pressure liquid chromatography)
BOP (bis(2-oxo-3-oxazolidinyl)phosphinic chloride);
EDCI (1-ethyl-3-[3-dimethylaminopropyl]carbodiimide hydrochloride)
HBTU (O-Benzotriazolel-yl-N,N,N′,N′-tetramethyluronium hexafluorophosphate)
DBU (1,8-diazabicyclo[5.4.0]undec-7-ene)
IPA (2-propanol)
NIS (N-iodosuccinimide)
NFPT (1-fluoropyridinium triflate)
DAST (diethylaminosulfur trifluoride)
BINAP (racemic-2,2′-bis(diphenylphosphino)-1,1′-binaphthyl)

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted, and all solvents are of the highest available purity unless otherwise indicated.

Microwave reaction was conducted with a Biotage microwave reactor.

¹H NMR spectra were recorded on either a Jeol ECX-400, or a Jeol JNM-LA300 spectrometer. Chemical shifts were expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Mass spectra were obtained with either Micromass, Quattro LC Triple Quadrupole Tandem Mass Spectometer, ESI or Agilent, 6110 Quadrupole LC/MS, ESI.

For preparative HPLC, ca 100 mg of a product was injected in 1 mL of DMSO onto a SunFire™ Prep C18 OBD 5 um 19×100 mm Column with a 10 min gradient from 10% CH₃CN to 90% CH₃CN in H₂O. Flash chromatography was carried using silica gel 60 (230-400 mesh, E. Merck). Most of the reactions were monitored by thin-layer chromatography on 0.25 mm silica gel plates (60E-254, E. Merck), visualized with UV light using a 5% ethanolic phosphomolybdic acid or p-anisaldehyde solution.

### Example 1

### Preparation of (+)-2-Fluoro-3-hydroxymorphinan TFA salt

### Step 1: Preparation of (+)-3-Hydroxy-N-(benzyloxycarbonyl)morphinan

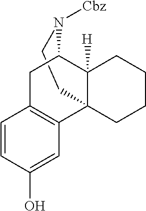

To (+)-3-hydroxymorphinan HBr (50.0 g, 154 mmol) and sodium hydroxide (12.3 g, 308 mmol) in a mixture of 1,4-dioxane (200 mL) and water (200 mL) was added Cbz-Cl (24.2 mL, 170 mmol) dropwise at rt. The reaction mixture was stirred vigorously at rt overnight. After the reaction was completed, water (200 mL) was added thereto. The resulting mixture was extracted with diethyl ether (500 mL×2). The combined organic phase was dried over MgSO₄, filtered, and evaporated under vacuum. Standing under high vacuum provided the title compound (57.7 g, 99%) as a light yellow solid. The compound was used for the next step without further purification.

¹H NMR (300 MHz, CDCl₃): δ 7.36-7.32 (m, 5H), 6.91m, 1H), 6.76 (s, 1H), 6.62 (m, 1H), 5.17-5.12 (m, 2H), 4.35 (d, J=29.25 Hz, 1H), 3.92-3.82 (m, 1H), 3.11-3.03 (m, 1H), 2.72-2.56 (m, 2H), 2.31-2.28 (m, 1H), 1.63-1.26 (m, 10H), 1.11-1.00 (m, 1H).

MH+ 378.

### Step 2: Preparation of (+)-2-Fluoro-3-hydroxy-N-(benzyloxycarbonyl)morphinan

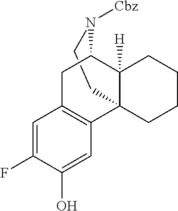

A mixture of (+)-3-hydroxy-N-(benzyloxycarbonyl)morphinan (1.13 g, 3 mmol) and NFPT (0.74 g, 3 mmol) in 1,1,2-trichloroethane (8 mL) was heated at 80° C. for 24 hours. The reaction mixture was evaporated to remove the solvent under vacuum. The residue was poured into water (50 mL) and extracted with DCM (50 mL). The combined organic phase was dried over MgSO₄ and evaporated under vacuum. The residue was purified by prep. reverse-phase HPLC (Gilson, C18 column) to provide the title compound (0.29 g, 24.5%).

MH+ 396.

### Step 3: Preparation of (+)-2-Fluoro-3-hydroxymorphinan TFA salt

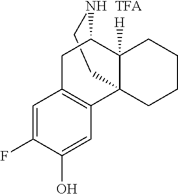

A part of the purified (+)-2-fluoro-3-hydroxy-N-(benzyloxycarbonyl)morphinan (224 mg, 0.566 mmol) was dissolved in EtOH (20 mL), and then 10% Pd on charcoal (45 mg) was added to the solution. The resulting mixture was stirred under hydrogen atmosphere at room temperature for 24 hrs. The reaction mixture thus obtained was filtered to remove the catalyst and evaporated under vacuum. The residue was purified by prep. reverse-phase HPLC (0.1% TFA added) to provide the title compound (153 mg, 72%).

¹H NMR (400 MHz, CD₃OD): δ 6.92-6.82 (m, 2H), 3.65-3.62 (br, 1H), 3.29-3.20 (m, 1H), 3.15-3.07 (m, 1H), 2.95-2.85 (m, 2H), 2.78-2.70 (m, 1H), 1.90-1.64 (m, 4H), 1.56-1.41 (m, 3H), 1.40-1.09 (m, 3H).

MH+ 262.

### Example 2

### Preparation of (+)-4-Chloro-2-fluoro-3-hydroxymorphinan TFA salt

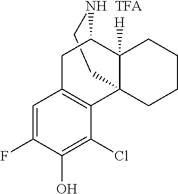

To a solution of crude (+)-2-fluoro-3-hydroxymorphinan TFA salt obtained in Example 1 (357 mg, 1.44 mmol) in glacial acetic acid (15 mL) under nitrogen atmosphere was added sulfuryl chloride (0.233 mL, 2.87 mmol) dropwise. The resulting reaction mixture was stirred overnight and evaporated under vacuum. The residue was purified by prep. reverse-phase HPLC (0.1% TFA added) to provide the title compound (346 mg, 59%).

¹H NMR (400 MHz, CDCl₃): δ 6.99-6.88 (m, 1H), 3.59 (br, 1H), 3.30-3.20 (m, 1H), 3.16-3.07 (m, 2H), 2.85 (d, J=13.6 Hz, 1H), 2.72 (br, 1H), 2.07-1.96 (m, 2H), 1.92-1.81 (m, 1H), 1.78-1.60 (m, 3H), 1.51-1.41 (m, 2H), 1.38-1.05 (m, 2H).

MH+ 296.

### Example 3

### Preparation of (+)-4-Bromo-2-fluoro-3-hydroxymorphinan TFA salt

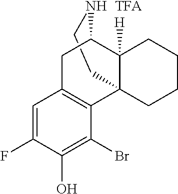

To a solution of crude (+)-2-fluoro-3-hydroxymorphinan TFA salt obtained in Example 1 (357 mg, 1.44 mmol) and TEA (0.95 mL, 7.2 mmol) in glacial acetic acid (15 mL) under nitrogen atmosphere was added dropwise bromine (0.07 mL) in acetic acid (1 mL). After stirring 0.5 hr at rt., the resulting reaction mixture was cooled to 0° C. Ammonium hydroxide solution (8.6 mL) was added to the reaction mixture with stirring. The precipitate thus obtained was filtered, washed with water, and purified by prep. reverse-phase HPLC (0.1% TFA added) to provide the title compound (461 mg, 70%).

¹H NMR (400 MHz, CDCl₃): δ 7.14-6.92 (m, 1H), 3.62 (br, 1H), 3.35-3.04 (m, 2H), 2.85 (d, J=13.6 Hz, 1H), 2.77-2.71 (m, 1H), 2.14-1.95 (m, 2H), 1.93-1.84 (m, 1H), 1.80-1.60 (m, 3H), 1.51-1.05 (m, 5H).

MH+ 340.

### Example 4

### Preparation of (+)-2,4-Dichloro-3-hydroxymorphinan TFA salt

### Step 1: Preparation of (+)-3-Hydroxymorphinan

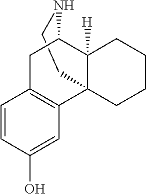

To a solution of (+)-3-hydroxymorphinan HBr (32.4 g, 100 mmol) in 1,4-dioxane (200 mL) was added sodium hydroxide (8.00 g, 200 mmol) in water (200 mL) at 0° C. The resulting reaction mixture was stirred for 30 min at r.t. and then EtOAc (100 mL) was added thereto. The mixture thus obtained was stirred for another 30 min and filtered. The filtered cake was dried under high vacuum to provide the title compound (21.9 g, 90%) as a yellow solid. The compound was used for the next step without further purification.

MH+ 244.

### Step 2: Preparation of (+)-2,4-Dichloro-3-hydroxymorphinan TFA salt

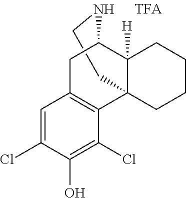

To a solution of (+)-3-hydroxymorphinan (0.973 g, 4 mmol) in glacial acetic acid (40 mL) under nitrogen atmosphere was added sulfuryl chloride (0.65 mL, 8 mmol) dropwise. The resulting reaction mixture was stirred overnight and evaporated under vacuum. The residue was purified by prep. reverse-phase HPLC (0.1% TFA added) to provide the title compound (0.334 g, 20%).

¹H NMR (400 MHz, CDCl₃): δ 7.14 (s, 1H), 3.61-3.57 (br, 2H), 3.29-3.23 (m, 1H), 3.18-3.08 (br, 2H), 2.70 (br, 1H), 2.05 (t, J=13.6 Hz, 2H), 1.85 (t, J=13.6 Hz, 1H), 1.70-1.60 (m, 2H), 1.50-1.41 (m, 2H), 1.31-1.07 (m, 3H).

MH+ 312.

### Example 5

### Preparation of (+)-4-Chloro-3-hydroxymorphinan TFA salt

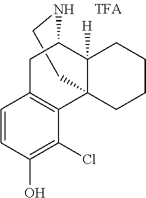

To a solution of (+)-2,4-dichloro-3-hydroxymorphinan (187 mg, 0.439 mmol) in MeOH (15 mL) was added 10% Pd on charcoal (150 mg). The mixture was stirred under hydrogen atmosphere at r.t. for 48 hrs. The resulting reaction mixture was filtered to remove the catalyst and evaporated under vacuum. The residue was purified by prep. reverse-phase HPLC (0.1% TFA added) to provide the title compound (67 mg, 39%).

¹H NMR (400 MHz, CDCl₃): δ 7.03 (d, J=8.4 Hz, 1H), 6.96 (d, J=8.4 Hz, 1H), 3.61-3.59 (br, 2H), 3.29 (dd, J=18.8, 6.0 Hz, 1H), 3.15-3.05 (br, 2H), 2.73 (br, 1H), 2.05-2.01 (m, 2H), 1.84 (t, J=13.6 Hz, 1H), 1.70-1.60 (m, 2H), 1.50-1.41 (m, 2H), 1.31-1.10 (m, 3H).

MH+ 278.

### Example 6

### Preparation of (+)-2,4-Dibromo-3-hydroxymorphinan TFA salt

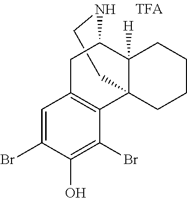

To a solution of (+)-3-hydroxymorphinan HBr (3.24 g, 10 mmol) and TEA (6.97 mL, 50 mmol) in glacial acetic acid (50 mL) under nitrogen atmosphere was added dropwise bromine (1 mL) in 5 mL acetic acid. After stirring 0.5 hr at r.t., the resulting reaction mixture was cooled to 0° C. Ammonium hydroxide solution (60 mL) was added to the reaction mixture with stirring. The precipitate was filtered, washed with water, and purified by prep. reverse-phase HPLC (0.1% TFA added) to provide the title compound (1.71 g, 33%).

¹H NMR (400 MHz, CDCl₃): δ 7.33 (s, 1H), 3.86 (d, J=14.2 Hz, 1H), 3.58 (br, 1H), 3.35-3.28 (m, 1H), 3.20-3.06 (br, 2H), 2.74 (br, 1H), 2.20-2.05 (m, 2H), 1.82 (t, J=13.8 Hz, 1H), 1.72-1.58 (m, 2H), 1.54-1.42 (m, 2H), 1.29-1.04 (m, 3H).

MH+ 402.

### Example 7

### Preparation of (+)-4-Bromo-3-hydroxymorphinan TFA salt

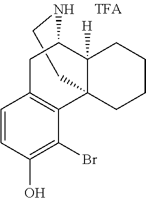

To a solution of 2,4-dibromo-3-hydroxymorphinan TFA salt obtained in Example 6 (1.39 g, 2.70 mmol) in MeOH (50 mL) was added 10% Pd on charcoal (700 mg) and stirred under hydrogen atmosphere at r.t. for 2 hrs. The resulting reaction mixture was filtered to remove the catalyst and evaporated under vacuum. The residue was purified by prep. reverse-phase HPLC (0.1% TFA added) to provide the title compound (0.798 g, 68%).

¹H NMR (400 MHz, CDCl₃): δ 7.06 (d, J=8.4 Hz, 1H), 6.96 (d, J=8.4 Hz, 1H), 3.84 (d, J=14.0 Hz, 1H), 3.58 (br, 1H), 3.32 (dd, J=18.8, 5.6 Hz, 1H), 3.16-3.09 (m, 2H), 2.74 (br, 1H), 2.09 (t, J=12.0 Hz, 2H), 1.83 (t, J=10.8 Hz, 1H), 1.69-1.58 (m, 2H), 1.50-1.42 (m, 2H), 1.27-1.09 (m, 3H).

MH+ 322.

### Example 8

### Preparation of (+)-4-Bromo-2-chloro-3-hydroxymorphinan TFA salt

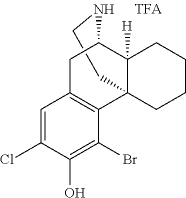

To a solution of 4-bromo-3-hydroxymorphinan TFA salt obtained in Example 7 (0.550 g, 1.26 mmol) in glacial acetic acid (13 mL) under nitrogen atmosphere was added sulfuryl chloride (0.204 mL, 2.52 mmol) dropwise. The resulting reaction mixture was stirred overnight and evaporated under vacuum. The residue was purified by prep. reverse-phase HPLC (0.1% TFA added) to provide the title compound (0.490 g, 83%).

¹H NMR (400 MHz, CDCl₃): δ 7.18 (s, 1H), 3.87 (d, J=13.2 Hz, 1H), 3.58 (br, 1H), 3.36-3.26 (m, 1H), 3.16-3.08 (br, 2H), 2.73 (br, 1H), 2.16-2.01 (m, 2H), 1.88-1.76 (m, 1H), 1.72-1.57 (m, 2H), 1.52-1.41 (m, 2H), 1.25-1.07 (m, 3H).

MH+ 358.

### Example 9

### Preparation of (+)-3-Hydroxy-2-iodomorphinan TFA salt

### Step 1: Preparation of (+)-3-Hydroxy-N-(tert-butyloxycarbonyl)morphinan

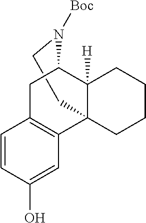

To (+)-3-hydroxymorphinan HBr (50.0 g, 154 mmol) and sodium hydroxide (13.6 g, 339 mmol) in a mixture of 1,4-dioxane (200 mL) and water (200 mL) was added di-tert-butyl dicarbonate (37.0 g, 167 mmol) at r.t. The resulting reaction mixture was stirred vigorously at rt overnight. After the reaction was completed, water (200 mL) was added thereto. The mixture thus obtained was extracted with EtOAc (500 mL×2).

The combined organic phase was dried over MgSO₄, filtered, and evaporated under vacuum. The residue was purified by flash column chromatography (Biotage SP1™) to provide the title compound (47.8 g, 90%) as a light yellow solid.

MH+ 344.

### Step 2: Preparation of (+)-3-Hydroxy-2-iodo-N-(tert-butyloxycarbonyl)morphinan

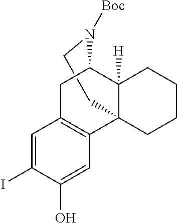

To a solution of (+)-3-hydroxy-N-(tert-butyloxycarbonyl)morphinan obtained in step 1 (8.59 g, 25 mmol) in DMF (125 mL) was added dropwise NIS (8.44 g, 37.5 mmol) in DMF (75 mL). The resulting reaction mixture was stirred at room temperature for 3 hrs, and then diluted EtOAc (500 mL). The resulting mixture was washed successively with brine, dried over MgSO₄, and evaporated under vacuum. The residue was purified by flash column chromatography (Biotage, silica) to provide the title compound (7.23 g, 15.4 mmol, 62%).

MH+ 470.

### Step 3: Preparation of (+)-3-Hydroxy-2-iodomorphinan TFA salt

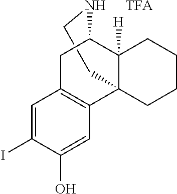

The mixture of (+)-3-hydroxy-2-iodo-N-(tert-butyloxycarbonyl)morphinan obtained in step 2 (345 mg, 0.735 mmol) and a HCl solution (5 mL, 4M in dioxane) was stirred at r.t. for 18 hrs. The resulting reaction mixture was evaporated under vacuum and the residue was purified by prep. reverse-phase HPLC (0.1% TFA added) to provide the title compound (247 mg, 70%).

¹H NMR (400 MHz, CDCl₃): δ 7.47 (s, 1H), 6.92 (s, 1H), 3.63 (br, 1H), 3.17-3.04 (m, 3H), 2.76 (br, 1H), 2.32 (d, J=13.6 Hz, 1H), 2.04 (d, J=11.6 Hz, 1H), 1.93 (t, J=12.8 Hz, 1H), 1.68 (d, J=12.8 Hz, 1H), 1.58 (d, J=13.2 Hz, 1H), 1.51-1.38 (m, 3H), 1.28-1.23 (m, 2H), 1.10-1.00 (m, 1H).

MH+ 370.

### Example 10

### Preparation of (+)-2,3-Dihydroxymorphinan TFA salt

### Step 1: Preparation of (+)-2,3-Dihydroxy-N-(benzyloxycarbonyl)morphinan

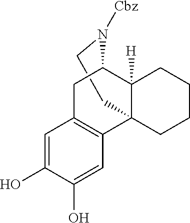

Solid 1-hydroxy-1,2-benziodoxol-3(1H)-one 1-oxide (IBX) (2.24 g, 8 mmol) was added to a solution of (+)-3-hydroxy-N-(benzyloxycarbonyl)morphinan obtained in step 1 of Example 1 (1.51 g, 4 mmol) in CHCl₃/MeOH (100 mL, 3/2, v/v) at −25° C. After stirring for 24 hrs, methanolic NaBH₄ (85.1 mg in 5 mL) was added to the resulting reaction mixture at −25° C. under vigorous stirring until the orange color disappeared (within 20 min). The reaction mixture thus obtained was acidified by acetic acid (2 mL), and then diluted with CHCl₃/MeOH (150 mL, 2/1, v/v). The resulting mixture was washed three times with PBS buffer solution (100 mL, pH 7.4, containing 10% sodium dithionite). The organic phase was dried over MgSO₄ and evaporated under vacuum. The residue was purified by prep. HPLC (Gilson, C18 column) to provide the title compound (0.229 g, 15%).

MH+ 394.

### Step 2: Preparation of (+)-2,3-Dihydroxymorphinan TFA salt

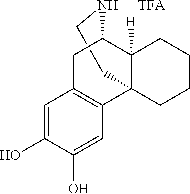

A part of the purified (+)-2,3-dihydroxy-N-(benzyloxycarbonyl)morphinan obtained in step 1 (229 mg, 0.58 mmol) was dissolved in EtOH (15 mL), and then 10% Pd on charcoal (50 mg) was added thereto. The resulting mixture was stirred under hydrogen atmosphere at r.t. overnight, filtered to remove the catalyst, and evaporated under vacuum. The residue was purified by prep. reverse-phase HPLC (0.1% TFA added) to provide the title compound (103 mg, 48%).

¹H NMR (400 MHz, CD₃OD): δ 6.77 (s, 1H), 6.59 (s, 1H), 3.62-3.60 (br, 1H), 3.18 (dd, J=18.8, 6.0 Hz, 1H), 3.08 (d, J=15.6 Hz, 1H), 2.83-2.74 (m, 2H), 2.35 (d, J=8.8 Hz, 1H), 1.85 (d, J=12.4 Hz, 1H), 1.78-1.70 (m, 2H), 1.56-1.28 (m, 6H), 1.20-1.10 (m, 1H).

MH+ 260.

### Example 11

### Preparation of (+)-3,4-(Methylenedioxy)morphinan TFA salt

### Step 1: Preparation of (+)-3,4-(Methylenedioxy)-N-(benzyloxycarbonyl)morphinan

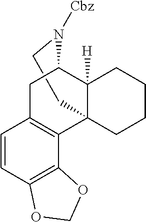

The mixture of 3,4-dihydroxy-N-(benzyloxycarbonyl)morphinan (purified from step 2 of Example 10, 150 mg, 0.381 mmol), K₂CO₃ (263 mg, 1.91 mmol) and diiodomethane (510 mg, 1.91 mmol) in acetone/DMF (5 mL, 1/1, v/v) was heated at 150° C. for 0.5 hr. The resulting reaction mixture was diluted with 1 M HCl aqueous solution (30 mL), and then extracted with EtOAc (30 mL×3). The combined EtOAc was washed with brine and evaporated under vacuum. The residue was purified by prep. HPLC (Gilson, C18 column) to provide the title compound (65 mg, 42%).

MH+ 406.

### Step 2: Preparation of (+)-3,4-(Methylenedioxy)morphinan TFA salt

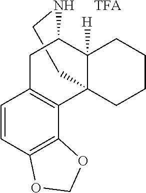

The purified (+)-3,4-(methylenedioxy)-N-(benzyloxycarbonyl)morphinan ontained in step 1 (65 mg, 0.16 mmol) was dissolved in MeOH (10 mL), and then 10% Pd on charcoal (30 mg) was added thereto. The resulting mixture was stirred under hydrogen atmosphere at r.t. for 3 hours, filtered to remove the catalyst, and evaporated under vacuum. The residue was purified by prep. reverse-phase HPLC (0.1% TFA added) to provide the title compound (34 mg, 55%).

¹H NMR (400 MHz, CDCl₃) δ 6.74 (d, J=8.0 Hz, 1H), 6.68 (d, J=8.4 Hz, 1H), 5.91 (s, 2H), 3.64 (br, 1H), 3.24-3.03 (m, 3H), 2.92-2.82 (m, 2H), 1.95 (d, J=12.0 Hz, 1H), 1.87-1.61 (m, 4H), 1.51-1.36 (m, 2H), 1.30-1.12 (m, 3H).

MH+ 272.

### Example 12

### Preparation of (+)-3-Hydroxy-2-methoxymorphinan TFA salt

### Step 1: Preparation of (+)-3-Hydroxy-2-iodo-N-(benzyloxycarbonyl)morphinan

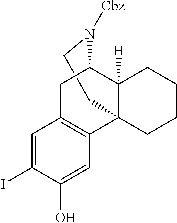

To a solution of (+)-3-hydroxy-N-(benzyloxycarbonyl)morphinan obtained in step 1 of Example 1 (3.77 g, 10 mmol) in DMF (50 mL) was added NIS (3.38 g, 15 mmol) in DMF (30 mL). The resulting reaction mixture was stirred at r.t. for 3 hrs. and then diluted EtOAc (300 mL). The resulting mixture was washed successively with brine, dried over MgSO₄, and evaporated under vacuum. The residue was purified by prep. HPLC (Gilson, C18 column) to provide the title compound (3.09 g, 61%).

MH+ 504.

### Step 2: Preparation of (+)-3-Hydroxy-2-methoxy-N-(benzyloxycarbonyl)morphinan

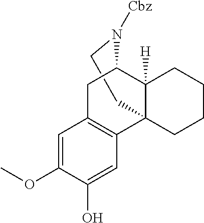

To a mixture of (+)-3-hydroxy-2-iodo-N-(benzyloxycarbonyl)morphinan obtained in step 1 (0.805 g, 1.6 mmol) and CuCl₂ (71.0 mg, 0.528 mmol) in DMF (6.4 mL) was added NaOMe (3.6 mL, 25% in MeOH1). The resulting reaction mixture was heated at 120° C. for 0.5 hr, and then filtered to remove solid particle. The resulting solution was purified by prep. reverse-phase HPLC (Gilson, C18 column) to provide the title compound (130 mg, 20%).

MH+ 408.

### Step 3: Preparation of (+)-3-Hydroxy-2-methoxymorphinan TFA salt

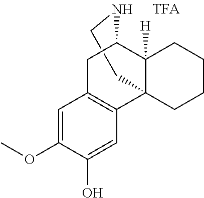

To a solution of (+)-3-hydroxy-2-methoxy-N-(benzyloxycarbonyl)morphinan obtained in step 2 (130 mg, 0.319 mmol) in MeOH (10 mL) was added 10% Pd on charcoal (40 mg). The resulting mixture was stirred under hydrogen atmosphere at r.t. overnight. The reaction mixture thus obtained was filtered to remove the catalyst and evaporated under vacuum. The residue was purified by prep. reverse-phase HPLC (0.1% TFA added) to provide the title compound (92 mg, 74%).

¹H NMR (400 MHz, CDCl₃): δ 6.83 (s. 1H), 6.62 (s, 1H), 3.88 (s, 3H), 3.64 (br, 1H), 3.16-3.02 (m, 3H), 2.81 (br, 1H), 2.31 (d, J=13.6 Hz, 1H), 2.01 (d, J=12.0 Hz, 1H), 1.86 (t, J=10.4 Hz, 1H), 1.67 (d, J=12.0 Hz, 1H), 1.57-1.26 (m, 6H), 1.16-1.05 (m, 1H).

MH+ 274.

### Example 13

### Preparation of (+)-2-Formyl-3-hydroxymorphinan TFA salt

### Step 1: Preparation of (+)-2-Formyl-3-hydroxy-N-(benzyloxycarbonyl)morphinan

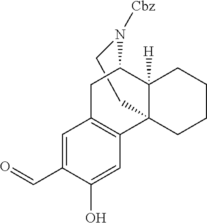

A mixture of (+)-3-hydroxy-N-(benzyloxycarbonyl)morphinan obtained in step 1 of Example 1 (5.00 g, 13.2 mmol), MgCl₂ (1.89 g, 19.8 mmol), TEA (4.6 mL, 33.0 mmol) and paraformaldehyde (3.98 g, 132 mmol) in acetonitrile (50 mL) was heated within a screw-capped vessel at 110° C. for 5 days. The resulting reaction mixture was filtered and washed with EtOAc (200 mL) and water (100 mL). The combined organic phase was dried over MgSO₄, filtered, and evaporated under vacuum. The residue was purified by flash column chromatography (Biotage SP1™) to provide the title compound (3.01 g, 56%) as a white solid.

¹H NMR (400 MHz, CDCl₃): δ 10.73 (s, 1H), 9.83 (s, 1H), 7.38-7.24 (m, 6H), 6.98 (s, 1H), 5.21-5.10 (m, 2H), 4.47-4.30 (m, 1H), 3.98-3.84 (m, 1H), 3.75 (t, J=6.4 Hz, 1H), 3.18-3.03 (m, 1H), 2.78-2.56 (m, 2H), 2.39-2.31 (m, 1H), 1.87-1.84 (m, 1H), 1.76-1.47 (m, 4H), 1.39-1.22 (m, 3H), 1.07-1.00 (m, 1H).

MH+ 406.

### Step 2: Preparation of (+)-2-Formyl-3-hydroxymorphinan TFA salt

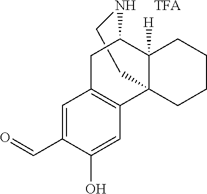

(+)-2-Formyl-3-hydroxy-N-(benzyloxycarbonyl)morphinan obtained in step 1 (250 mg, 0.617 mmol) was subjected to hydrogenation (balloon) on 10% Pd/C (25 mg) in IPA (10 mL) at r.t. After the reaction was completed, the resulting reaction mixture was filtered through a Celite, and washed with IPA (20 mL). The combined IPA solution was evaporated under vacuum. The residue was purified by prep. reverse-phase HPLC (0.1% TFA added) to provide the title compound (75 mg, 31%) as a yellow solid.

¹H NMR (400 MHz, CD₃OD): δ 7.18 (s, 1H), 6.79 (s, 1H), 5.55 (s, 1H), 3.65 (q, J=2.8 Hz, 1H), 3.30-3.21 (m, 1H), 3.08 (dd, J=13.2, 3.2 Hz, 1H), 2.89 (d, J=18.8 Hz, 1H), 2.73 (td, J=13.2, 3.6 Hz, 1H), 2.40 (d, J=14.0 Hz, 1H), 1.88 (d, J=12.8 Hz, 1H), 1.78 (td, J=14.0, 4.8 Hz, 1H), 1.70 (d, J=11.2 Hz, 1H), 1.58-1.48 (m, 3H), 1.44-1.28 (m, 4H), 1.12-1.08 (m, 1H).

MH+ 272.

### Example 14

### Preparation of ((+)-3-Hydroxymorphinan)-2-carboxylic acid TFA salt

### Step 1: Preparation of ((+)-3-Hydroxy-N-(benzyloxycarbonyl)morphinan)-2-carboyxlic acid

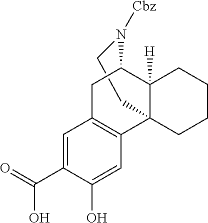

To a solution of (+)-2-formyl-3-hydroxy-N-(benzyloxycarbonyl)morphinan obtained in step 1 of Example 13 (1.20 g, 2.96 mmol) in acetone (50 mL) was added KMnO₄ (0.702 g, 4.44 mmol) at 0° C. The resulting reaction mixture was allowed to warm to room temperature and stirred overnight. Oxalic acid (2.0 g, 22.2 mmol) was added to the reaction mixture. After stirring for 0.5 hour, the resulting mixture was filtered and diluted with DCM (100 mL). The organic phase was washed with brine, dried over MgSO₄, and evaporated under vacuum. The residue was purified by prep. reverse-phase HPLC (Gilson, C18 column) to provide the title comlound (574 mg, 46%).

MH+ 422.

### Step 2: Preparation of ((+)-3-Hydroxymorphinan)-2-carboyxlic acid TFA salt

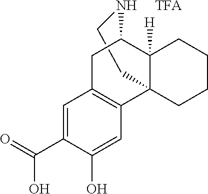

The purified ((+)-3-hydroxy-N-(benzyloxycarbonyl)morphinan)-2-carboyxlic acid obtained in step 1 (574 mg, 1.36 mmol) was dissolved in EtOH (30 mL), and then 10% Pd on charcoal (200 mg) was added thereto. The resulting mixture was stirred under hydrogen atmosphere at r.t. overnight. The reaction mixture thus obtained was filtered to remove the catalyst and evaporated under vacuum. The residue was purified by prep. reverse-phase HPLC (0.1% TFA added) to provide the title compound (415 mg, 76%).

¹H NMR (400 MHz, CD₃OD): δ 7.71 (s, 1H), 6.94 (s, 1H), 3.69 (m, 1H), 3.61 (br, 1H), 3.10 (dd, J=12.4, 4.4 Hz, 1H), 2.95 (d, J=18.8 Hz, 1H), 2.73 (td, J=13.2, 3.6 Hz, 1H), 2.45 (d, J=14.0 Hz, 1H), 1.94-1.90 (m, 1H), 1.83 (td, J=14.0, 4.8 Hz, 1H), 1.76-1.68 (m, 1H), 1.64-1.51 (m, 3H), 1.49-1.36 (m, 2H), 1.34-1.22 (m, 1H), 1.14-1.02 (m, 1H).

MH+ 288.

### Example 15

### Preparation of (+)-2-(Difluoromethyl)-3-hydroxymorphinan TFA salt

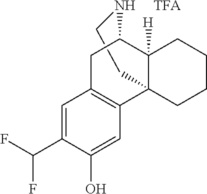

To a solution of (+)-2-formyl-3-hydroxy-N-(benzyloxycarbonyl)morphinan obtained in step 1 of Example 13 (339 mg, 0.836 mmol) in DCM (10 mL) was added DAST (0.331 mL, 2.51 mmol). The resulting reaction mixture was stirred at r.t. overnight and then quenched by addition of saturated NaHCO₃ aqueous solution (4-5 mL). The resulting mixture was washed successively with water, dried over MgSO₄, and evaporated under vacuum. The crude residue was dissolved in EtOH (30 mL), and then 10% Pd on charcoal (350 mg) was added thereto. The mixture thus obtained was stirred under hydrogen atmosphere at r.t. overnight, filtered to remove the catalyst, and evaporated under vacuum. The residue was purified by prep. reverse-phase HPLC (0.1% TFA added) to provide the title compound (133 mg, 39%).

¹H NMR (400 MHz, CD₃OD): δ 7.29 (s, 1H), 7.18-6.76 (m, 2H), 3.69-3.66 (m, 1H), 3.26-3.22 (m, 1H), 3.13-3.07 (m, 1H), 2.93 (d, J=18.8 Hz, 1H), 2.77-2.69 (m, 1H), 2.40 (d, J=14.0 Hz, 1H), 1.96-1.67 (m, 3H), 1.62-1.26 (m, 6H), 1.15-1.07 (m, 1H). MH+ 294.

### Example 16

### Preparation of (+)-3-Hydroxy-2-(hydroxymethyl)morphinan TFA salt

### Step 1: Preparation of (+)-3-Hydroxy-2-(hydroxymethyl)-N-(benzyloxycarbonyl)morphinan

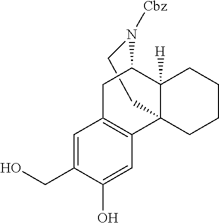

To a solution of (+)-2-formyl-3-hydroxy-N-(benzyloxycarbonyl)morphinan obtained in step 1 of Example 13 (949 mg, 2.34 mmol) in EtOH (25 mL) was added NaBH₄ (500 mg, 13.2 mmol). The resulting reaction mixture was stirred at r.t. overnight, and then diluted with 0.5 M HCl aqueous solution (30 mL). The resulting mixture was extracted with DCM (60 mL) and then the organic phase was evaporated under vacuum. The residue was purified by prep HPLC (Gilson, C18 column) to provide the title compound (482 mg, 51%).

MH+ 408.

### Step 2: Preparation of (+)-3-Hydroxy-2-(hydroxymethyl)morphinan TFA salt

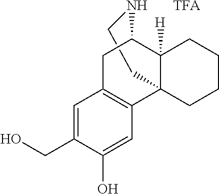

A part of the purified (+)-3-hydroxy-2-(hydroxymethyl)-N-(benzyloxycarbonyl)morphinan obtained in step 1 (211 mg, 0.518 mmol) was dissolved in EtOH (10 mL), and then 10% Pd on charcoal (80 mg) was added thereto. The resulting mixture was stirred under hydrogen atmosphere at r.t. overnight. The reaction mixture thus obtained was filtered to remove the catalyst and evaporated under vacuum. The residue was purified by prep. reverse-phase HPLC (0.1% TFA added) to provide the title compound (109 mg, 54%).

¹H NMR (400 MHz, CD₃OD): δ 6.98 (s, 1H), 6.77 (s, 1H), 4.71 (d, J=4.8 Hz, 2H), 3.61 (br, 1H), 3.22-3.16 (m, 1H), 3.09-3.05 (m, 1H), 2.93 (d, J=18.8 Hz, 1H), 2.76 (td, J=13.2, 3.2 Hz, 1H), 2.37 (d, J=13.6 Hz, 1H), 1.93 (d, J=12.8 Hz, 1H), 1.86-1.78 (m, 1H), 1.75-1.67 (m, 1H), 1.59-1.25 (m, 6H), 1.16-1.07 (m, 1H).

MH+ 274.

### Example 17

### Preparation of (+)-2-(Azepan-1-yl)-3-hydroxymorphinan TFA salt

### Step 1: Preparation of (+)-3-Hydroxy-2-iodomorphinan

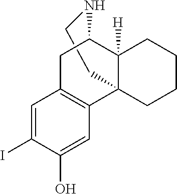

A solution of I₂ (5.08 g, 20.0 mmol) and KI (4.98 g, 30.0 mmol) in water (200 mL) was added dropwise to a stirred solution of (+)-3-hydroxymorphinan HBr (3.24 g, 10 mmol) in 2 N NaOH (65 mL) and water (135 mL). After stirring for 30 min, the resulting reaction mixture was neutralized with dry ice, and the yellowish precipitate was separated by filtration, washed with water, and dried to provide the title compound (3.48 g, 94%) as a yellow solid.

¹H NMR (400 MHz, DMSO-d₆): δ 7.34 (s, 1H), 6.73 (s, 1H), 2.90-2.84 (m, 2H), 2.55 (d, J=17.2 Hz, 1H), 2.35 (t, J=12.4 Hz, 1H), 2.09 (d, J=13.2 Hz, 1H), 1.60-1.53 (m, 2H), 1.43-1.40 (m, 2H), 1.29-1.11 (m, 6H), 0.86-0.83 (m, 1H).

MH+ 370.

### Step 2: Preparation of (+)-3-Hydroxy-2-iodo-N-(benzyloxycarbonyl)morphinan

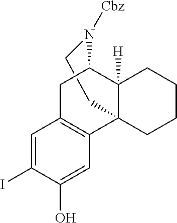

To (+)-3-hydroxy-2-iodomorphinan (29) (3.26 g, 8.83 mmol) and sodium hydroxide (706 mg, 17.7 mmol) in a mixture of 1,4-dioxane (100 mL) and water (100 mL) was added Cbz-Cl (1.39 mL, 9.71 mmol) dropwise at r.t. The resulting reaction mixture was stirred vigorously at r.t. overnight. After the reaction was completed, water (100 mL) was added the reaction mixture. The mixture thus obtained was extracted with diethyl ether (100 mL×2). The combined organic phase was dried over MgSO₄, filtered, and evaporated under vacuum. The residue was purified by flash column chromatography (Biotage SP1™) to provide the title compound (3.36 g, 76%) as a white solid.

MH+ 504.

### Step 3: Preparation of (+)-2-Iodo-3-methoxy-N-(benzyloxycarbonyl)morphinan

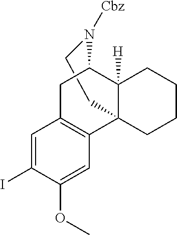

To (+)-3-hydroxy-2-iodo-N-(benzyloxycarbonyl)morphinan obtained in step 2 (8.36 g, 16.6 mmol) and K₂CO₃ (4.59 g, 33.2 mmol) in acetone (100 mL) was added iodomethane (1.55 mL, 24.9 mmol) at r.t. The resulting reaction mixture was stirred at r.t. overnight. After the reaction was completed, water (200 mL) was added thereto. The mixture thus obtained was extracted with EtOAc (200 mL×2). The combined organic phase was dried over MgSO₄, filtered, and evaporated under vacuum. The residue was purified by flash column chromatography (Biotage SP1™) to provide the title compound (6.73 g, 78%) as a white solid.

MH+ 518.

### Step 4: Preparation of (+)-2-(Azepan-1-yl)-3-methoxy-N-(benzyloxycarbonyl)morphinan

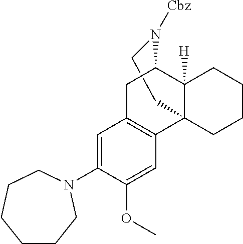

To a solution of (+)-2-iodo-3-methoxy-N-(benzyloxycarbonyl)morphinan obtained in step 3 (1.00 g, 1.93 mmol) in THF (10 mL) were added hexamethyleneimine (260 μL, 2.32 mmol), NatBuO (260 mg, 2.71 mmol), Pd₂(dba)₃ (17.7 mg, 0.0193 mmol), BINAP (18.0 mg, 0.0289 mmol), and 15-crown-5 (540 μL, 2.71 mmol). The resulting reaction mixture was irradiated in a microwave reactor (Biotage) for 30 min at 165° C. After the reaction was completed, water (10 mL) was added thereto. The mixture thus obtained was extracted with EtOAc (15 mL×2). The combined organic phase was dried over MgSO₄, filtered, and evaporated under vacuum. The residue was purified by flash column chromatography (Biotage SP1™) to provide the title compound (553 mg, 59%) as a white solid.

MH+ 489.

### Step 5: Preparation of (+)-2-(Azepan-1-yl)-3-hydroxymorphinan TFA salt

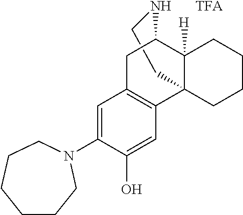

To a solution of (+)-2-(azepan-1-yl)-3-methoxy-N-(benzyloxycarbonyl)morphinan obtained in step 4 (553 mg, 1.13 mmol) in DCM (5 mL) was added BBr₃ solution (1M in DCM, 3.4 mL, 3.40 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (21 mg, 4.1%) as a colorless gum.

¹H NMR (400 MHz, CD₃OD): δ 7.45 (s, 1H), 7.04 (s, 1H), 3.77-3.76 (m, 4H), 3.71 (q, J=2.8 Hz, 1H), 3.32-3.25 (m, 1H), 3.12 (dd, J=13.2, 3.2 Hz, 1H), 3.00 (d, J=19.2 Hz, 1H), 2.71 (td, J=13.6, 3.6 Hz, 1H), 2.38 (d, J=14.0 Hz, 1H), 2.68-2.22 (m, 4H), 1.95 (dt, J=12.4, 2.8 Hz, 1H), 1.88-1.80 (m, 5H), 1.69 (d, J=12.4 Hz, 1H), 1.61-1.42 (m, 5H), 1.24-1.21 (m, 1H), 1.05-1.01 (m, 1H).

MH+ 341.

The following compounds of Examples 18 to 24 were obtained by repeating the procedure of Example 17.

### Example 18

### Preparation of (+)-3-Hydroxy-2-(methylamino)morphinan TFA salt

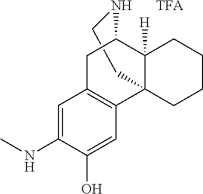

¹H NMR (400 MHz, CD₃OD): δ 7.22 (s, 1H), 7.00 (s, 1H), 3.71 (dd, J=5.6, 3.2 Hz, 1H), 3.37-3.28 (m, 1H), 3.12 (dd, J=12.8, 3.6 Hz, 1H), 3.02-2.97 (m, 4H), 2.72 (td, J=13.6, 3.6 Hz, 1H), 2.39 (d, J=14.8 Hz, 1H), 1.97-1.94 (m, 1H), 1.84 (td, J=13.6, 4.8 Hz, 1H), 1.70 (d, J=12.4 Hz, 1H), 1.60-1.43 (m, 5H), 1.29-1.23 (m, 1H), 1.11-1.03 (m, 1H).

MH+ 273.

### Example 19

### Preparation of (+)-3-Hydroxy-2-(4-methylpiperidin-1-yl)morphinan TFA salt

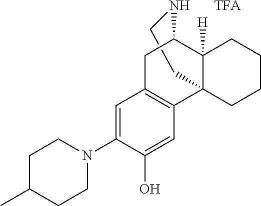

¹H NMR (400 MHz, CD₃OD): δ 7.52 (s, 1H), 7.03 (s, 1H), 3.78-3.66 (m, 5H), 3.34-3.28 (m, 2H), 3.13 (dd, J=13.2, 3.2 Hz, 1H), 3.05 (d, J=19.2 Hz, 1H), 2.72 (td, J=13.6, 3.6 Hz, 1H), 2.39 (d, J=14.4 Hz, 1H), 2.04-1.96 (m, 2H), 1.86 (td, J=13.6, 4.8 Hz, 2H), 1.78-1.68 (m, 3H), 1.60-1.40 (m, 5H), 1.27-1.21 (m, 1H), 1.07 (d, J=6.4 Hz, 3H), 1.04-0.99 (m, 1H).

MH+ 341.

### Example 20

### Preparation of (+)-2-(tert-Butylamino)-3-hydroxymorphinan TFA salt

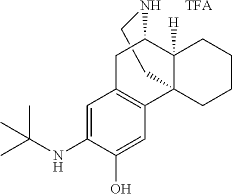

¹H NMR (400 MHz, CD₃OD): δ 7.14 (s, 1H), 7.04 (s, 1H), 3.73 (dd, J=6.0, 3.2 Hz, 1H), 3.45-3.28 (m, 1H), 3.14 (dd, J=13.6, 3.6 Hz, 1H), 3.02 (d, J=19.6 Hz, 1H), 2.73 (td, J=13.6, 3.6 Hz, 1H), 2.41 (d, J=14.0 Hz, 1H), 1.98-1.95 (m, 1H), 1.86 (td, J=14.0, 4.4 Hz, 1H), 1.72 (d, J=12.8 Hz, 1H), 1.63-1.37 (m, 14H), 1.30-1.21 (m, 1H), 1.12-1.02 (m, 1H).

MH+ 315.

### Example 21

### Preparation of (+)-3-Hydroxy-2-(piperidin-1-yl)morphinan TFA salt

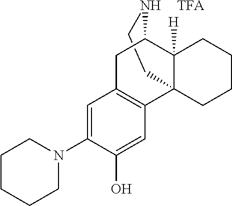

¹H NMR (400 MHz, CD₃OD) 7.43 (s, 1H), 7.03 (s, 1H), 3.72-3.70 (m, 1H), 3.67-3.64 (m, 4H), 3.12 (dd, J=13.2, 3.2 Hz, 1H), 3.00 (d, J=19.6 Hz, 1H), 2.71 (td, J=13.6, 4.0 Hz, 1H), 2.39 (d, J=14.0 Hz, 1H), 2.06-2.01 (m, 4H), 1.95 (dt, J=12.4, 2.8 Hz, 1H), 1.88-1.68 (m, 4H), 1.61-1.39 (m, 6H), 1.27-1.17 (m, 1H), 1.08-0.98 (m, 1H).

MH+ 327.

### Example 22

### Preparation of (+)-3-Hydroxy-2-(pyrrolidin-1-yl)morphinan TFA salt

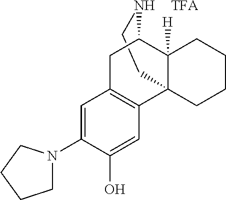

¹H NMR (400 MHz, CD₃OD) 7.39 (s, 1H), 7.05 (s, 1H), 3.81-3.80 (m, 4H), 3.74-3.71 (m, 1H), 3.14 (dd, J=13.6, 3.2 Hz, 1H), 3.01 (d, J=19.2 Hz, 1H), 2.73 (td, J=13.6, 3.6 Hz, 1H), 2.41 (d, J=14.8 Hz, 1H), 2.30-2.25 (m, 5H), 1.97 (dt, J=12.4, 2.8 Hz, 1H), 1.86 (td, J=13.6, 4.8 Hz, 1H) 1.72 (d, J=12.8 Hz, 1H), 1.63-1.41 (m, 5H), 1.31-1.23 (m, 1H), 1.11-1.04 (m, 1H).

MH+ 312.

### Example 23

### Preparation of (+)-2-Ethylamino-3-hydroxymorphinan TFA salt

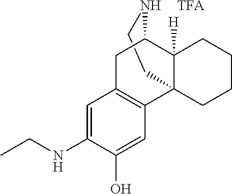

¹H NMR (400 MHz, CD₃OD): δ 7.24 (s, 1H), 7.01 (s, 1H), 3.72 (dd, J=6.0, 3.2 Hz, 1H), 3.43 (q, J=3.2 Hz, 2H), 3.32-3.26 (m, 1H), 3.13 (dd, J=13.6, 3.6 Hz, 1H), 3.01 (d, J=19.2 Hz, 1H), 2.72 (td, J=13.6, 3.6 Hz, 1H), 2.40 (d, J=14.0 Hz, 1H), 1.96 (dt, J=12.4, 2.8 Hz, 1H), 1.85 (td, J=14.0, 3.2 Hz, 1H), 1.71 (d, J=13.2 Hz, 1H), 1.61-1.38 (m, 5H), 1.35 (t, J=7.6 Hz, 3H), 1.30-1.22 (m, 1H), 1.10-1.00 (m, 1H).

MH+ 287.

### Example 24

### Preparation of (+)-3-Hydroxy-2-(4-methylpiperazin-1-yl)morphinan 2TFA salt

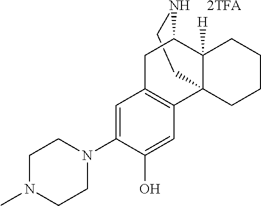

¹H NMR (400 MHz, CD₃OD): δ 6.89 (s, 1H), 6.80 (s, 1H), 4.19-4.13 (m, 2H), 3.66-3.64 (m, 1H), 3.55 (d, J=12.8 Hz, 2H), 3.48-3.42 (m, 2H), 3.35-3.21 (m, 5H), 3.08 (dd, J=13.2, 3.6 Hz, 1H), 2.89 (d, J=19.2 Hz, 1H), 2.74 (td, J=13.6, 3.6 Hz, 1H), 2.38 (d, J=13.6 Hz, 1H), 1.89-1.85 (m, 1H), 1.77 (td, J=13.6, 4.8 Hz, 1H), 1.71-1.68 (m, 1H), 1.56-1.48 (m, 3H), 1.43-1.27 (m, 4H), 1.13-1.08 (m, 1H).

MH+ 342.

### Example 25

### Preparation of (+)-2-(4-Chlorophenylamino)-3-hydroxymorphinan TFA salt

### Step 1: Preparation of (+)-2-(4-Chlorophenylamino)-3-methoxy-N-(benzyloxycarbonyl)morphinan

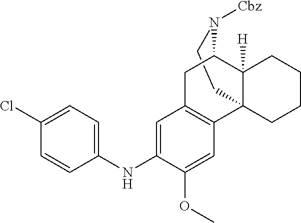

To a solution of (+)-2-iodo-3-methoxy-N-(benzyloxycarbonyl)morphinan obtained in step 3 of Example 17 (1.00 g, 1.93 mmol) in THF (10 mL) were added 4-chloroaniline (246 mg, 1.93 mmol), NatBuO (186 mg, 1.93 mmol), (dppf)PdCl₂.CH₂Cl₂ (63.0 mg, 0.0772 mmol), and dppf (128 mg, 0.232 mmol). The resulting reaction mixture was irradiated in a microwave reactor (Biotage) for 30 min at 155° C. After the reaction was completed, water (10 mL) was added thereto. The resulting mixture was extracted with EtOAc (15 mL×2). The combined organic phase was dried over MgSO₄, filtered, and evaporated under vacuum. The residue was purified by flash column chromatography (Biotage SP1™) to provide the title compound (572 mg, 57%) as a white solid.

MH+ 517.

### Step 2: Preparation of (+)-2-(4-Chlorophenylamino)-3-hydroxymorphinan TFA salt

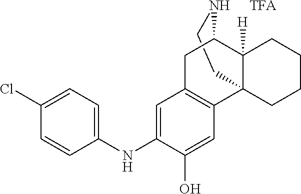

To a solution of (+)-2-(4-chlorophenylamino)-3-methoxy-N-(benzyloxycarbonyl)morphinan obtained in step 1 (572 mg, 1.11 mmol) in DCM (10 mL) was added BBr₃ solution (1M in DCM, 6.7 mL, 6.70 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (335 mg, 75%) as a brown solid.

¹H NMR (400 MHz, CD₃OD): δ 7.15 (d, J=8.8 Hz, 2H), 6.99-6.97 (m, 3H), 6.81 (s, 1H), 3.64-3.61 (m, 1H), 3.21 (dd, J=19.2, 6.0 Hz, 1H), 3.12-3.06 (m, 1H), 2.84-2.72 (m, 2H), 2.45-2.37 (m, 1H), 1.94-1.73 (m, 3H), 1.58-1.33 (m, 6H), 1.23-1.06 (m, 1H).

MH+ 369.

The following compounds of Examples 26 to 71 were obtained by repeating the procedure of Example 25.

### Example 26

### Preparation of (+)-3-Hydroxy-2-(4-hydroxyphenylamino)morphinan TFA salt

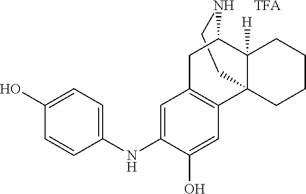

¹H NMR (400 MHz, CD₃OD) 7.00-6.98 (m, 2H), 6.74-6.72 (m, 4H), 3.59-3.56 (m, 1H), 3.14 (dd, J=18.8, 6.0 Hz, 1H), 3.05 (dd, J=13.2, 3.2 Hz, 1H), 2.83-2.72 (m, 2H), 2.35 (d, J=8.4 Hz, 1H), 1.84 (dt, J=12.0, 2.8 Hz, 1H), 1.78-1.70 (m, 2H), 1.56-1.34 (m, 6H), 1.20-1.11 (m, 1H).

MH+ 351.

### Example 27

### Preparation of (+)-2-(3,5-Dimethylphenylamino)-3-hydroxymorphinan TFA salt

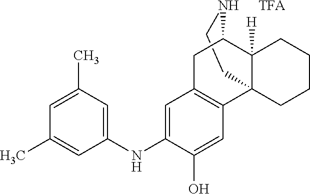

¹H NMR (400 MHz, CD₃OD): δ 6.97 (s, 1H), 6.78 (s, 2H), 6.67 (s, 1H), 6.51 (s, 1H), 3.62-3.60 (m, 1H), 3.20 (dd, J=19.2, 6.4 Hz, 1H), 3.07 (dd, J=13.2, 3.2 Hz, 1H), 2.85-2.78 (m, 2H), 2.37 (d, J=10.4 Hz, 1H), 2.22 (s, 6H), 1.86 (dt, J=12.0, 2.8 Hz, 1H), 1.80-1.70 (m, 2H), 1.56-1.36 (m, 6H), 1.23-1.16 (m, 1H).

MH+ 363.

### Example 28

### Preparation of (+)-3-Hydroxy-2-(4-methylphenylamino)morphinan TFA salt

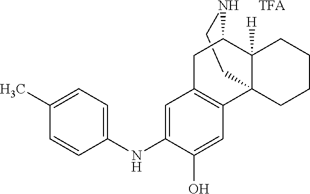

¹H NMR (400 MHz, CD₃OD): δ 7.04 (d, J=8.4 Hz, 2H), 6.97 (d, J=8.4 Hz, 2H), 6.92 (s, 1H), 6.76 (s, 1H), 3.61-3.58 (m, 1H), 3.17 (dd, J=19.2, 6.4 Hz, 1H), 3.07 (dd, J=12.4, 4.8 Hz, 1H), 2.84-2.76 (m, 2H), 2.36 (d, J=10.8 Hz, 1H), 2.25 (s, 3H), 1.85 (dt, J=12.4, 3.2 Hz, 1H), 1.79-1.69 (m, 2H), 1.55-1.34 (m, 6H), 1.21-1.14 (m, 1H).

MH+ 349.

### Example 29

### Preparation of (+)-2-(4-Fluorophenylamino)-3-hydroxymorphinan TFA salt

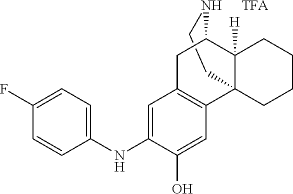

¹H NMR (400 MHz, CD₃OD): δ 7.22-7.18 (m, 2H), 7.08-7.03 (m, 2H), 6.99 (s, 1H), 6.85 (s, 1H), 3.66-3.62 (m, 1H), 3.21 (dd, J=19.2, 6.4 Hz, 1H), 3.09 (dd, J=13.2, 3.2 Hz, 1H), 2.86 (d, J=18.8 Hz, 1H), 2.78 (td, J=13.2, 3.6 Hz, 1H), 2.38 (d, J=13.6 Hz, 1H), 1.90 (dt, J=12.0, 3.2 Hz, 1H), 1.79 (td, J=13.6, 4.8 Hz, 1H), 1.71 (d, J=13.2 Hz, 1H), 1.59-1.27 (m, 6H), 1.19-1.09 (m, 1H).

MH+ 353.

### Example 30

### Preparation of (+)-3-Hydroxy-2-(phenylamino)morphinan TFA salt

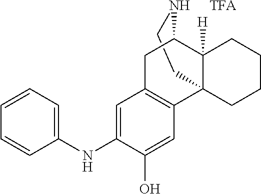

¹H NMR (400 MHz, CD₃OD): δ 7.20 (t, J=8.0 Hz, 2H), 7.04 (dd, J=8.8, 0.8 Hz, 2H), 7.01 (s, 1H), 6.83 (t, J=7.2 Hz, 1H), 6.79 (s, 1H), 3.61-3.59 (m, 1H), 3.20 (dd, J=19.2, 6.4 Hz, 1H), 3.08 (dd, J=12.0, 4.8 Hz, 1H), 2.86-2.77 (m, 2H), 2.38 (d, J=10.8 Hz, 1H), 1.86 (dt, J=12.0, 3.2 Hz, 1H), 1.80-1.70 (m, 2H), 1.57-1.37 (m, 6H), 1.24-1.14 (m, 1H).

MH+ 335.

### Example 31

### Preparation of (+)-3-Hydroxy-2-(4-methoxyphenylamino)morphinan TFA salt

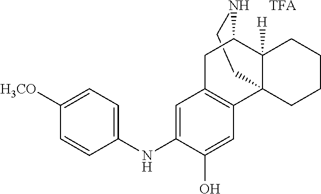

¹H NMR (400 MHz, CD₃OD): δ 7.05 (d, J=8.8 Hz, 2H), 6.85 (d, J=8.8 Hz, 2H), 6.76 (s, 1H), 6.74 (s, 1H), 3.75 (s, 3H), 3.58 (dd, J=5.6, 3.2 Hz, 1H), 3.15 (dd, J=19.2, 6.4 Hz, 1H), 3.06 (dd, J=12.4, 3.2 Hz, 1H), 2.85-2.72 (m, 2H), 2.37 (d, J=10.8 Hz, 1H), 1.84 (dt, J=12.0, 3.2 Hz, 1H), 1.75-1.70 (m, 2H), 1.55-1.35 (m, 6H), 1.22-1.14 (m, 1H).

MH+ 365.

### Example 32

### Preparation of (+)-2-(4-Aminophenyamino)-3-hydroxymorphinan TFA salt

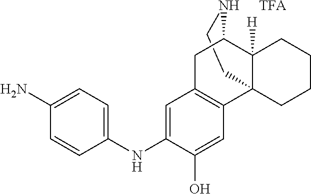

¹H NMR (400 MHz, CD₃OD): δ 7.17 (d, J=8.8 Hz, 2H), 7.06 (d, J=8.8 Hz, 2H), 7.03 (s, 1H), 6.84 (s, 1H), 6.79 (s, 1H), 3.65-3.63 (m, 1H), 3.22 (dd, J=18.4, 6.0 Hz, 1H), 3.09 (dd, J=12.8, 3.2 Hz, 1H), 2.87-2.78 (m, 2H), 2.39 (d, J=13.2 Hz, 1H), 1.88 (d, J=12.4 Hz, 1H), 1.82-1.71 (m, 2H), 1.58-1.27 (m, 6H), 1.19-1.15 (m, 1H).

MH+ 350.

### Example 33

### Preparation of (+)-2-(4-Bromophenylamino)-3-hydroxymorphinan TFA salt

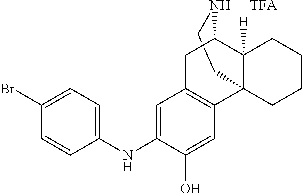

¹H NMR (400 MHz, CD₃OD): δ 7.28 (d, J=8.8 Hz, 2H), 7.00 (s, 1H), 6.93 (d, J=8.8 Hz, 2H), 6.81 (s, 1H), 3.62 (q, J=2.8 Hz, 1H), 3.22 (dd, J=19.2, 6.4 Hz, 1H), 3.09 (dd, J=13.2, 3.2 Hz, 1H), 2.84-2.74 (m, 2H), 2.39 (d, J=12.8 Hz, 1H), 1.86 (d, J=12.4 Hz, 1H), 1.80-1.72 (m, 2H), 1.58-1.33 (m, 6H), 1.23-1.14 (m, 1H).

MH+ 413.

### Example 34

### Preparation of (+)-3-Hydroxy-2-(pyridin-2-ylamino)morphinan 2TFA salt

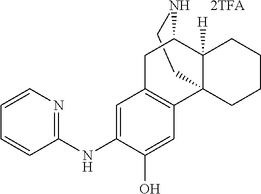

¹H NMR (400 MHz, CD₃OD): δ 7.98-7.94 (m, 1H), 7.85-7.83 (m, 1H), 7.14-7.12 (m, 2H), 7.02-6.94 (m, 2H), 3.70-3.69 (m, 1H), 3.26-3.19 (m, 1H), 3.17-3.11 (m, 1H), 2.84-2.74 (m, 2H), 2.46-2.38 (m, 1H), 1.93-1.71 (m, 3H), 1.62-1.35 (m, 6H), 1.16-1.12 (m, 1H).

MH+ 336.

### Example 35

### Preparation of (+)-3-Hydroxy-2-(4-(trifluoromethyl)phenylamino)morphinan TFA salt

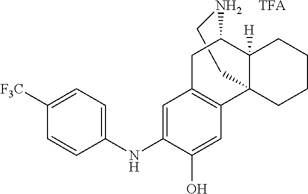

¹H NMR (400 MHz, CD₃OD): δ 7.41 (d, J=8.4 Hz, 2H), 7.09 (s, 1H), 7.02 (d, J=8.4 Hz, 2H), 6.86 (s, 1H), 3.64 (q, J=2.8 Hz, 1H), 3.25 (dd, J=19.2, 6.8 Hz, 1H), 3.10 (dd, J=13.2, 3.2 Hz, 1H), 2.88-2.78 (m, 2H), 2.40 (d, J=12.8 Hz, 1H), 1.88 (d, J=12.8 Hz, 1H), 1.82-1.72 (m, 2H), 1.60-1.34 (m, 6H), 1.27-1.14 (m, 1H).

MH+ 403.

### Example 36

### Preparation of (+)-3-Hydroxy-2-((3,4-methylendioxy)phenylamino)morphinan TFA salt

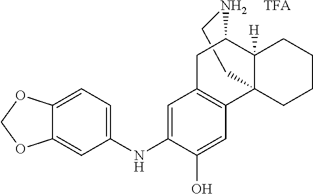

¹H NMR (400 MHz, CD₃OD): δ 6.81 (s, 1H), 6.75 (s, 1H), 6.71-6.69 (m, 1H), 6.66 (d, J=2.0 Hz, 1H), 6.54 (dd, J=8.0, 2.0 Hz, 1H), 5.87 (s, 2H), 3.59 (q, J=2.8 Hz, 1H), 3.16 (dd, J=19.2, 6.4 Hz, 1H), 3.06 (dd, J=13.2, 3.2 Hz, 1H), 2.84-2.74 (m, 2H), 2.36 (d, J=9.2 Hz, 1H), 1.85 (d, J=12.4 Hz, 1H), 1.79-1.70 (m, 2H), 1.55-1.35 (m, 6H), 1.21-1.13 (m, 1H).

MH+ 379.

### Example 37

### Preparation of (+)-2((3,4-Ethylenedioxy)phenylamino)-3-hydroxymorphinan TFA salt

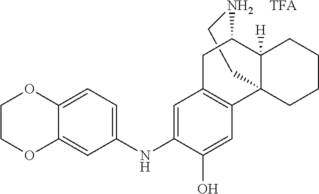

¹H NMR (400 MHz, CD₃OD): δ 6.83 (s, 1H), 6.74 (s, 1H), 6.72 (d, J=7.6 Hz, 1H), 6.58 (td, J=8.4, 2.8 Hz, 2H), 4.19 (dd, J=8.8, 6.0 Hz, 4H), 3.59 (q, J=2.8 Hz, 1H), 3.17 (dd, J=19.2, 6.4 Hz, 1H), 3.06 (dd, J=13.2, 3.2 Hz, 1H), 2.84-2.74 (m, 2H), 2.37 (d, J=7.2 Hz, 1H), 1.84 (d, J=12.0 Hz, 1H), 1.74-1.71 (m, 2H), 1.56-1.35 (m, 6H), 1.24-1.14 (m, 1H).

MH+ 393.

### Example 38

### Preparation of (+)-2-(2-Fluorophenylamino)-3-hydroxymorphinan TFA salt

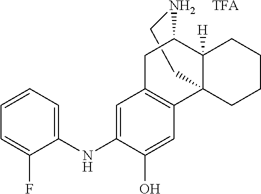

¹H NMR (400 MHz, CD₃OD): δ 7.23 (td, J=8.0, 1.2 Hz, 1H), 7.10-7.01 (m, 2H), 6.93 (s, 1H), 6.87-6.82 (m, 2H), 3.62 (q, J=2.8 Hz, 1H), 3.21 (dd, J=19.2, 6.4 Hz, 1H), 3.08 (dd, J=13.2, 3.2 Hz, 1H), 2.85-2.77 (m, 2H), 2.38 (d, J=12.4 Hz, 1H), 1.87 (d, J=12.4 Hz, 1H), 1.81-1.71 (m, 2H), 1.57-1.33 (m, 6H), 1.22-1.16 (m, 1H).

MH+ 353.

### Example 39

### Preparation of (+)-2-(3-Fluorophenylamino)-3-hydroxymorphinan TFA salt

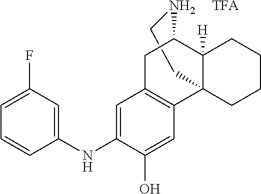

¹H NMR (400 MHz, CD₃OD): δ 7.17-12 (m, 1H), 7.04 (s, 1H), 6.83 (s, 1H), 6.79-6.77 (m, 1H), 6.69 (dt, J=11.6, 2.4 Hz, 1H), 6.50-6.45 (m, 1H), 3.63 (q, J=2.8 Hz, 1H), 3.23 (dd, J=19.2, 6.4 Hz, 1H), 3.09 (dd, J=13.2, 3.2 Hz, 1H), 2.87-2.78 (m, 2H), 2.41-2.38 (m, 1H), 1.87 (d, J=12.4 Hz, 1H), 1.81-1.72 (m, 2H), 1.58-1.31 (m, 6H), 1.24-1.14 (m, 1H).

MH+ 353.

### Example 40

### Preparation of (+)-2-(2,4-Dimethoxyphenylamino)-3-hydroxymorphinan TFA salt

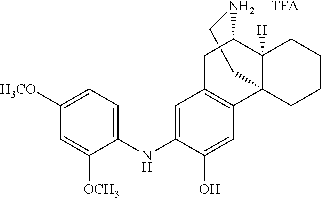

¹H NMR (400 MHz, CD₃OD): δ 7.16 (br s, 1H), 6.75 (br s, 2H), 6.60 (s, 1H), 6.48 (br s, 1H), 3.83 (s, 3H), 3.79 (s, 3H), 3.59 (q, J=2.8 Hz, 1H), 3.15 (dd, J=28.8, 7.2 Hz, 1H), 3.06 (dd, J=13.2, 3.2 Hz, 1H), 2.79 (td, J=13.2, 3.6 Hz, 2H), 2.36 (d, J=10.4 Hz, 1H), 1.85 (d, J=12.4 Hz, 1H), 1.78-1.69 (m, 2H), 1.55-1.27 (m, 6H), 1.20-1.11 (m, 1H).

MH+ 395.

### Example 41

### Preparation of (+)-3-Hydroxy-2-(2-methylphenylamino)morphinan TFA salt

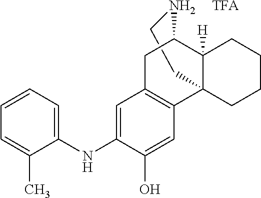

¹H NMR (400 MHz, CD₃OD): δ 7.16 (t, J=7.2 Hz, 2H), 7.09 (t, J=7.6 Hz, 1H), 6.88 (td, J=7.2, 1.2 Hz, 1H), 6.78 (s, 1H), 6.67 (s, 1H), 3.59 (q, J=2.8 Hz, 1H), 3.15 (dd, J=19.2, 6.4 Hz, 1H), 3.07 (dd, J=13.2, 3.2 Hz, 1H), 2.84-2.72 (m, 2H), 2.38-2.36 (m, 1H), 2.23 (s, 3H), 1.86-1.83 (m, 3H), 1.56-1.35 (m, 6H), 1.21-1.12 (m, 1H).

MH+ 349.

### Example 42

### Preparation of (+)-3-Hydroxy-2-(2-methoxyphenylamino)morphinan TFA salt

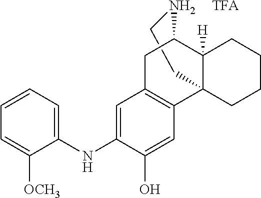

¹H NMR (400 MHz, CD₃OD): δ 7.26-7.22 (m, 1H), 7.06 (s, 1H), 6.96-6.94 (m, 1H), 6.87-6.82 (m, 2H), 6.79 (s, 1H), 3.88 (s, 3H), 3.62 (q, J=3.2 Hz, 1H), 3.22 (dd, J=18.8, 5.6 Hz, 1H), 3.07 (dd, J=13.2, 3.2 Hz, 1H), 2.85-2.78 (m, 2H), 2.38 (d, J=11.2 Hz, 1H), 1.88-1.72 (m, 3H), 1.57-1.36 (m, 6H), 1.27-1.14 (m, 1H). MH+ 365.

### Example 43

### Preparation of (+)-3-Hydroxy-2-(2-hydroxyphenylamino)morphinan TFA salt

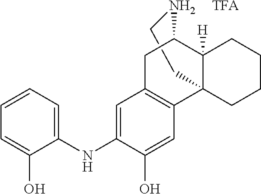

¹H NMR (400 MHz, CD₃OD): δ 7.22-7.20 (m, 1H), 6.95 (s, 1H), 6.82-6.80 (m, 1H), 6.77-6.73 (m, 3H), 3.60 (q, J=2.8 Hz, 1H), 3.19 (dd, J=19.2, 6.4 Hz, 1H), 3.06 (dd, J=12.0, 3.6 Hz, 1H), 2.84-2.78 (m, 2H), 2.36 (d, J=8.4 Hz, 1H), 1.87-1.69 (m, 3H), 1.55-1.31 (m, 6H), 1.19-1.15 (m, 1H).

MH+ 351.

### Example 44

### Preparation of (+)-3-Hydroxy-2-(3-hydroxyphenylamino)morphinan TFA salt

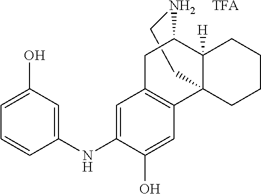

¹H NMR (400 MHz, CD₃OD): δ 7.03 (s, 2H), 7.00 (s, 1H), 6.79 (s, 2H), 6.51 (s, 1H), 3.61 (q, J=2.8 Hz, 1H), 3.19 (dd, J=19.2, 6.4 Hz, 1H), 3.06 (dd, J=12.0, 3.6 Hz, 1H), 2.80-2.76 (m, 2H), 2.36 (d, J=8.4 Hz, 1H), 1.87-1.69 (m, 3H), 1.55-1.31 (m, 6H), 1.19-1.15 (m, 1H).

MH+ 351.

### Example 45

### Preparation of (+)-2-(2,4-Dihydroxyphenylamino)-3-hydroxymorphinan TFA salt

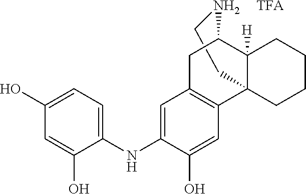

¹H NMR (400 MHz, CD₃OD): δ 6.99 (d, J=8.4 Hz, 1H), 6.74 (s, 1H), 6.55 (s, 1H), 6.39 (s, 1H), 6.28 (d, J=8.0 Hz, 1H), 3.57 (q, J=2.8 Hz, 1H), 3.13 (dd, J=19.2, 6.0 Hz, 1H), 3.04 (dd, J=12.8, 3.2 Hz, 1H), 2.82-2.70 (m, 2H), 2.35 (d, J=8.8 Hz, 1H), 1.85-1.69 (m, 3H), 1.55-1.35 (m, 6H), 1.17-1.13 (m, 1H).

MH+ 367.

### Example 46

### Preparation of (+)-2-(4-Hydroxyphenylamino)-3-hydroxymorphinan TFA salt

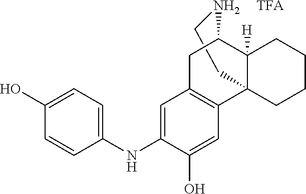

¹H NMR (400 MHz, CD₃OD): δ 6.88-6.83 (m, 2H), 6.76-6.66, (m, 3H), 6.54 (s, 1H), 3.59 (q, J=2.8 Hz, 1H), 3.12-3.06 (m, 2H), 2.76-2.72 (m, 2H), 2.40 (d, J=12.4 Hz, 1H), 1.88-1.71 (m, 3H), 1.59-1.31 (m, 6H), 1.16-1.11 (m, 1H).

MH+ 352.

### Example 47

### Preparation of (+)-2-(2,6-Dihydroxyphenylamino)-3-hydroxymorphinan TFA salt

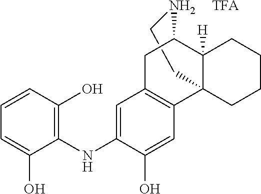

¹H NMR (400 MHz, CD₃OD): δ 6.87 (d, J=8.0 Hz, 1H), 6.72 (s, 1H), 6.41 (d, J=8.0 Hz, 2H), 6.12 (s, 1H), 3.54 (q, J=2.8 Hz, 1H), 3.11-3.00 (m, 2H), 2.79 (td, J=13.2, 3.6 Hz, 1H), 2.67 (d, J=18.8 Hz, 1H), 2.36 (d, J=9.6 Hz, 1H), 1.82-1.68 (m, 3H), 1.53-1.36 (m, 6H), 1.19-1.09 (m, 1H).

MH+ 367.

### Example 48

### Preparation of (+)-2-(2-Chlorophenylamino)-3-hydroxymorphinan TFA salt

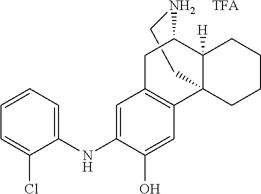

¹H NMR (400 MHz, CD₃OD): δ 7.35 (dd, J=8.0, 1.2 Hz, 1H), 7.24 (dd, J=8.0, 1.2 Hz, 1H), 7.15 (td, J=8.0, 1.6 Hz, 1H), 7.03 (s, 1H), 6.84 (s, 1H), 6.81 (td, J=8.0, 1.2 Hz, 1H), 3.63 (q, J=2.8 Hz, 1H), 3.24 (dd, J=19.2, 6.4 Hz, 1H), 3.09 (dd, J=13.2, 3.2 Hz, 1H), 2.86-2.78 (m, 2H), 2.40 (d, J=12.8 Hz, 1H), 1.88 (d, J=12.4 Hz, 1H), 1.81-1.72 (m, 2H), 1.59-1.31 (m, 6H), 1.24-1.15 (m, 1H).

MH+ 369.

### Example 49

### Preparation of (+)-2-(2-Ethylphenylamino)-3-hydroxymorphinan TFA salt

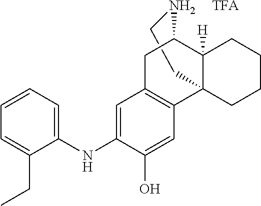

¹H NMR (400 MHz, CD₃OD): δ 7.21-7.18 (m, 2H), 7.11 (td, J=7.6, 1.2 Hz, 1H), 6.95 (td, J=7.6, 1.2 Hz, 1H), 6.77 (s, 1H), 6.54 (s, 1H), 3.58 (q, J=2.8 Hz, 1H), 3.14 (dd, J=19.2, 6.4 Hz, 1H), 3.06 (dd, J=13.2, 3.2 Hz, 1H), 2.80-2.71 (m, 2H), 2.62 (q, J=7.6 Hz, 2H), 2.39-2.37 (m, 1H), 1.84 (d, J=12.4 Hz, 1H), 1.75-1.71 (m, 2H), 1.56-1.35 (m, 7H), 1.19 (t, J=7.6 Hz, 3H).

MH+ 363.

### Example 50

### Preparation of (+)-3-Hydroxy-2-(2-isopropylphenylamino)morphinan TFA salt

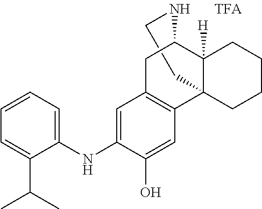

¹H NMR (400 MHz, CD₃OD): δ 7.30 (dd, J=7.6, 1.2 Hz, 1H), 7.19 (dd, J=8.0, 1.2 Hz, 1H), 7.11 (td, J=8.0, 1.6 Hz, 1H), 7.03 (t, J=7.2 Hz, 1H), 6.76 (s, 1H), 6.54 (s, 1H), 3.57 (q, J=2.8 Hz, 1H), 3.20-3.04 (m, 3H), 2.79 (td, J=13.2, 3.6 Hz, 1H), 2.71 (d, J=18.8 Hz, 1H), 2.37 (d, J=10.4 Hz, 1H), 1.84 (d, J=12.0 Hz, 1H), 1.78-1.70 (m, 2H), 1.56-1.38 (m, 7H), 1.21 (d, J=6.8 Hz, 6H).

MH+ 377.

### Example 51

### Preparation of (+)-2-(2-t-Butylphenylamino)-3-hydroxymorphinan TFA salt

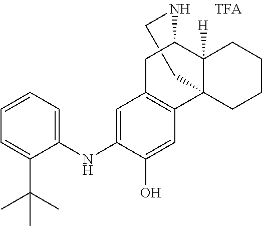

¹H NMR (400 MHz, CD₃OD): δ 7.41 (dd, J=8.0, 1.6 Hz, 1H), 7.27 (dd, J=8.0, 1.6 Hz, 1H), 7.14 (td, J=7.6, 1.6 Hz, 1H), 7.01 (td, J=7.6, 1.6 Hz, 1H), 6.76 (s, 1H), 6.55 (s, 1H), 3.56 (q, J=2.8 Hz, 1H), 3.15-3.04 (m, 2H), 2.80-2.68 (m, 2H), 2.38 (d, J=10.4 Hz, 1H), 1.83 (d, J=12.4 Hz, 1H), 1.74-1.70 (m, 2H), 1.56-1.35 (m, 15H), 1.24-1.17 (m, 1H).

MH+ 391.

### Example 52

### Preparation of (+)-3-Hydroxy-2-(2-(trifluoromethyl)phenylamino)morphinan TFA salt

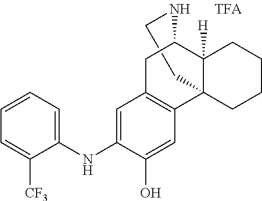

¹H NMR (400 MHz, CD₃OD): δ 7.56 (d, J=7.6 Hz, 1H), 7.45-7.38 (m, 2H), 7.01 (s, 1H), 6.97 (t, J=7.6 Hz, 1H), 6.84 (s, 1H), 3.63 (q, J=2.8 Hz, 1H), 3.22 (dd, J=19.2, 6.4 Hz, 1H), 3.09 (dd, J=13.2, 3.2 Hz, 1H), 2.86-2.78 (m, 2H), 2.39 (d, J=12.8 Hz, 1H), 1.88 (d, J=12.8 Hz, 1H), 1.82-1.72 (m, 2H), 1.59-1.31 (m, 6H), 1.24-1.15 (m, 1H).

MH+ 403.

### Example 53

### (+)-2-(4-Ethylphenylamino)-3-hydroxymorphinan TFA salt

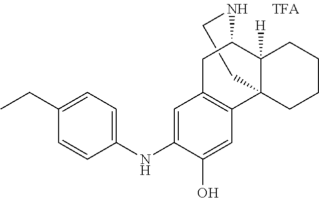

¹H NMR (400 MHz, CD₃OD): δ 7.07 (d, J=8.4 Hz, 2H), 6.99 (d, J=8.8 Hz, 2H), 6.94 (s, 1H), 6.77 (s, 1H), 3.60 (q, J=2.8 Hz, 1H), 3.18 (dd, J=19.2, 6.4 Hz, 1H), 3.07 (dd, J=13.2, 3.2 Hz, 1H), 2.84-2.76 (m, 2H), 2.56 (q, J=7.6 Hz, 2H), 2.37 (d, J=8.8 Hz, 1H), 1.85 (d, J=12.4 Hz, 1H), 1.78-1.71 (m, 2H), 1.56-1.35 (m, 7H), 1.19 (t, J=7.6 Hz, 3H).

MH+ 363.

### Example 54

### (+)-3-Hydroxy-2-(4-isopropylphenylamino)morphinan TFA salt

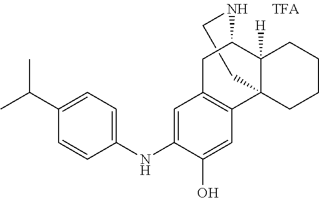

¹H NMR (400 MHz, CD₃OD): δ 7.10 (d, J=8.8 Hz, 2H), 7.00 (d, J=8.4 Hz, 2H), 6.95 (s, 1H), 6.77 (s, 1H), 3.60 (q, J=2.8 Hz, 1H), 3.18 (dd, J=19.2, 6.0 Hz, 1H), 3.07 (dd, J=13.2, 3.2 Hz, 1H), 2.86-2.76 (m, 3H), 2.37 (d, J=9.2 Hz, 1H), 1.85 (d, J=12.0 Hz, 1H), 1.79-1.71 (m, 2H), 1.56-1.36 (m, 7H), 1.21 (d, J=6.8 Hz, 6H).

MH+ 377.

### Example 55

### Preparation of (+)-2-(3-Chloro-2-hydroxyphenylamino)-3-hydroxymorphinan TFA salt

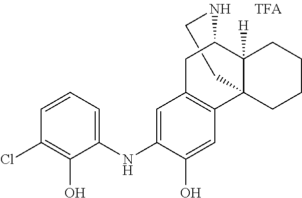

¹H NMR (400 MHz, CD₃OD): δ 7.12 (dd, J=8.0, 1.6 Hz, 1H), 6.97 (s, 1H), 6.82 (dd, J=8.0, 1.6 Hz, 1H), 6.80 (s, 1H), 6.74 (t, J=8.0 Hz, 1H), 3.61 (q, J=2.8 Hz, 1H), 3.21 (dd, J=19.2, 6.4 Hz, 1H), 3.08 (dd, J=13.2, 3.2 Hz, 1H), 2.85-2.77 (m, 2H), 2.38 (d, J=12.0 Hz, 1H), 1.87 (d, J=12.4 Hz, 1H), 1.84-1.70 (m, 2H), 1.56-1.33 (m, 6H), 1.22-1.15 (m, 1H).

MH+ 385.

### Example 56

### Preparation of (+)-2-(5-Fluoro-2-hydroxyphenylamino)-3-hydroxymorphinan TFA salt

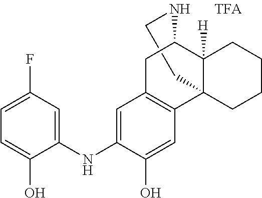

¹H NMR (400 MHz, CD₃OD): δ 7.05 (s, 1H), 6.90 (dd, J=10.8, 3.2 Hz, 1H), 6.81 (s, 1H), 6.72 (dd, J=8.4, 5.6 Hz, 1H), 6.36 (td, J=8.4, 2.8 Hz, 1H), 3.63 (q, J=2.8 Hz, 1H), 3.24 (dd, J=18.8, 6.0 Hz, 1H), 3.08 (dd, J=13.2, 3.2 Hz, 1H), 2.89-2.78 (m, 2H), 2.38 (d, J=12.4 Hz, 1H), 1.88 (d, J=12.4 Hz, 1H), 1.85-1.70 (m, 2H), 1.57-1.33 (m, 6H), 1.22-1.16 (m, 1H).

MH+ 369.

### Example 57

### Preparation of (+)-2-(3-Fluoro-2-hydroxyphenylamino)-3-hydroxymorphinan TFA salt

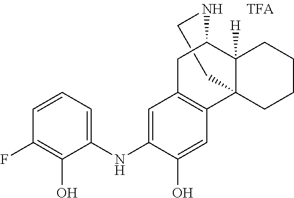

¹H NMR (400 MHz, CD₃OD): δ 6.85-6.79 (m, 3H), 6.76-6.72 (m, 2H), 3.59 (q, J=2.8 Hz, 1H), 3.17 (dd, J=19.2, 6.0 Hz, 1H), 3.07 (dd, J=13.2, 3.2 Hz, 1H), 2.83-2.76 (m, 2H), 2.36 (d, J=10.8 Hz, 1H), 1.85 (d, J=12.4 Hz, 1H), 1.76-1.69 (m, 2H), 1.55-1.35 (m, 6H), 1.18-1.14 (m, 1H).

MH+ 369.

### Example 58

### Preparation of (+)-3-Hydroxy-2-(4-(trifluoromethoxy)phenylamino)morphinan TFA salt

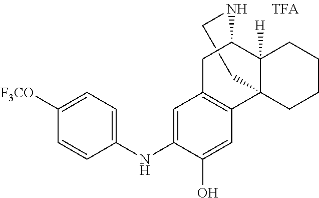

¹H NMR (400 MHz, CD₃OD): δ 7.10-7.01 (m, 5H), 6.82 (s, 1H), 3.62 (q, J=2.8 Hz, 1H), 3.21 (dd, J=19.2, 6.4 Hz, 1H), 3.09 (dd, J=13.2, 3.2 Hz, 1H), 2.85-2.77 (m, 2H), 2.38 (d, J=12.0 Hz, 1H), 1.87 (d, J=12.4 Hz, 1H), 1.81-1.71 (m, 2H), 1.57-1.33 (m, 6H), 1.22-1.16 (m, 1H).

MH+ 419.

### Example 59

### Preparation of (+)-3-Hydroxy-2-(2-(trifluoromethoxy)phenylamino)morphinan TFA salt

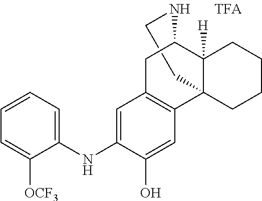

¹H NMR (400 MHz, CD₃OD): δ 7.31 (dd, J=8.0, 1.6 Hz, 1H), 7.24 (dt, J=8.0, 1.6 Hz, 1H), 7.19 (td, J=8.0, 1.6 Hz, 1H), 7.03 (s, 1H), 6.87 (td, J=8.0, 1.6 Hz, 1H), 6.84 (s, 1H), 3.63 (q, J=2.8 Hz, 1H), 3.23 (dd, J=19.2, 6.4 Hz, 1H), 3.09 (dd, J=13.2, 3.2 Hz, 1H), 2.85-2.78 (m, 2H), 2.39 (d, J=12.8 Hz, 1H), 1.88 (d, J=12.4 Hz, 1H), 1.82-1.71 (m, 2H), 1.58-1.34 (m, 6H), 1.23-1.16 (m, 1H).

MH+ 419.

### Example 60

### Preparation of (+)-2-(Biphenyl-2-ylamino)-3-hydroxymorphinan TFA salt

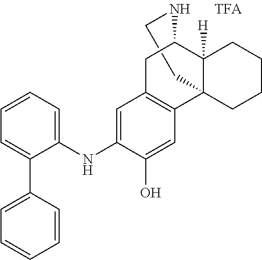

¹H NMR (400 MHz, CD₃OD): δ 7.40-7.31 (m, 5H), 7.28-7.21 (m, 3H), 6.99 (t, J=7.6 Hz, 1H), 6.83 (s, 1H), 6.68 (s, 1H), 3.58 (q, J=2.8 Hz, 1H), 3.13 (dd, J=19.2, 6.0 Hz, 1H), 3.04 (dd, J=13.2, 3.2 Hz, 1H), 2.78-2.71 (m, 2H), 2.32 (d, J=11.2 Hz, 1H), 1.83 (d, J=12.0 Hz, 1H), 1.80-1.68 (m, 2H), 1.54-1.29 (m, 6H), 1.17-1.09 (m, 1H).

MH+ 411.

### Example 61

### Preparation of (+)-2-(2-Carbamoylphenylamino)-3-hydroxymorphinan TFA salt

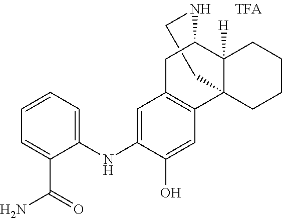

¹H NMR (400 MHz, CD₃OD): δ 7.62 (dd, J=8.0, 1.2 Hz, 1H), 7.28 (td, J=8.4, 1.6 Hz, 1H), 7.22 (dd, J=8.0, 1.2 Hz, 1H), 7.10 (s, 1H), 6.84 (s, 1H), 6.86 (td, J=8.0, 1.2 Hz, 1H), 3.63 (q, J=2.8 Hz, 1H), 3.23 (dd, J=18.8, 6.4 Hz, 1H), 3.09 (dd, J=13.2, 3.6 Hz, 1H), 2.86-2.78 (m, 2H), 2.39 (d, J=12.0 Hz, 1H), 1.87 (d, J=12.0 Hz, 1H), 1.81-1.72 (m, 2H), 1.59-1.30 (m, 6H), 1.23-1.15 (m, 1H).

MH+ 378.

### Example 62

### Preparation of (+)-2-(2-Benzylphenylamino)-3-hydroxymorphinan TFA salt

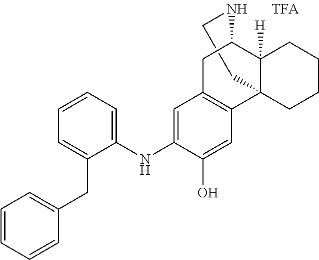

¹H NMR (400 MHz, CD₃OD): δ 7.25-7.09 (m, 8H), 6.98 (td, J=7.6, 1.6 Hz, 1H), 6.71 (s, 1H), 6.59 (s, 1H), 3.56 (q, J=2.8 Hz, 1H), 3.13-3.03 (m, 2H), 2.77 (td, J=13.2, 3.6 Hz, 1H), 2.69 (d, J=18.8 Hz, 1H), 2.34 (d, J=7.2 Hz, 1H), 1.82 (d, J=12.4 Hz, 1H), 1.77-1.69 (m, 2H), 1.54-1.33 (m, 6H), 1.19-1.13 (m, 1H).

MH+ 425.

### Example 63

### Preparation of (+)-2-(3,4-Dimethoxyphenylamino)-3-hydroxymorphinan TFA salt

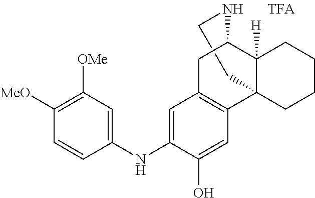

¹H NMR (400 MHz, CD₃OD): δ 7.27 (d, J=8.4 Hz, 1H), 7.09 (d, J=2.4 Hz, 1H), 7.12 (s, 1H), 6.97 (dd, J=10.8, 2.4 Hz, 1H), 6.87 (s, 1H), 3.82 (s, 3H), 3.79 (s, 3H), 3.66-3.63 (m, 1H), 3.25 (d, J=6.0 Hz 1H), 3.11 (dd, J=12.0, 3.4 Hz, 1H), 2.77-2.74 (m, 2H), 2.39 (s, 1H), 1.89 (d, J=12.4 Hz, 1H), 1.74 (dd, J=13.6, 4.4 Hz, 1H), 1.70 (s, 1H), 1.59-1.49 (m, 3H), 1.46-1.39 (m, 3H), 1.17 (dd, J=12.8, 4.0 Hz, 1H).

MH+ 395.

### Example 64

### Preparation of (+)-2-(2,5-Dichlorophenylamino)-3-hydroxymorphinan TFA salt

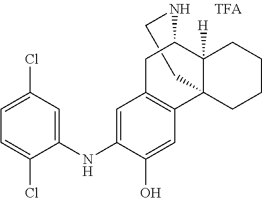

¹H NMR (400 MHz, CD₃OD): δ 7.29 (d, J=8.8 Hz, 1H), 7.04 (s, 1H), 6.98 (d, J=2.4 Hz, 1H), 6.89 (s, 1H), 6.75 (dd, J=8.4, 2.4 Hz, 1H), 3.96 (s, 1H), 3.65 (dd, J=3.2, 5.6 Hz, 1H), 3.24 (d, J=6.4 Hz, 1H), 3.11 (dd, J=13.2, 4.4 Hz, 1H), 2.91 (s, 1H), 2.86 (s, 1H), 2.83 (d, J=3.6 Hz, 1H), 2.39 (s, 1H), 1.89 (td, J=12.0, 2.8 Hz, 1H), 1.77 (dd, J=13.6, 4.8 Hz, 1H), 1.73 (s, 1H), 1.61-1.57 (m, 2H), 1.53-1.50 (m, 2H), 1.46-1.37 (m, 3H), 1.18 (dd, J=12.4, 3.6 Hz, 2H).

MH+ 403.

### Example 65

### Preparation of (+)-2-(3,4-Dichlorophenylamino)-3-hydroxymorphinan TFA salt

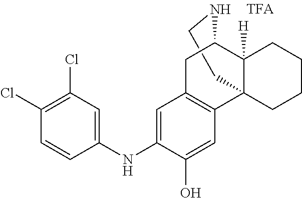

¹H NMR (400 MHz, CD₃OD): δ 7.25 (d, J=8.8 Hz, 1H), 7.04 (d, J=2.8 Hz, 1H), 7.00 (s, 1H), 6.87 (dd, J=8.8, 2.8 Hz, 1H), 6.84 (s, 1H), 3.65-3.63 (m, 1H), 3.24 (d, J=6.4 Hz 1H), 3.10 (dd, J=12.4, 3.2 Hz, 1H), 2.87-2.77 (m, 2H), 2.39 (s, 1H), 1.88 (d, J=12.4 Hz, 1H), 1.76 (dd, J=13.6, 4.4 Hz, 1H), 1.72 (s, 1H), 1.59-1.49 (m, 3H), 1.44-1.38 (m, 3H), 1.18 (dd, J=12.8, 4.0 Hz, 1H).

MH+ 403.

### Example 66

### Preparation of (+)-3-Hydroxy-2-(quinolin-8-ylphenylamino)morphinan TFA salt

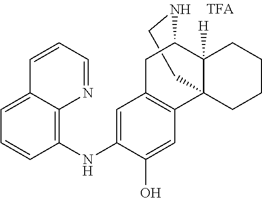

¹H NMR (400 MHz, CD₃OD): δ 8.82 (d, J=3.2 Hz, 1H), 8.33 (dd, J=8.4, 1.6 Hz, 1H), 7.56 (dd, J=8.4, 4.4 Hz, 1H), 7.51 (dd, J=8.0, 1.6 Hz, 1H), 7.47 (t, J=7.6 Hz, 1H), 7.34 (dd, J=7.6, 1.2 Hz, 1H), 7.31 (s, 1H), 6.89 (s, 1H), 3.66-3.65 (m, 1H), 3.33 (d, J=2.4 Hz, 1H), 3.10 (dd, J=12.4, 3.2 Hz, 1H), 2.92-2.87 (m, 2H), 2.42 (s, 1H), 1.90 (d, J=12.4 Hz, 1H), 1.76 (dd, J=13.6, 4.4 Hz, 1H), 1.72 (s, 1H), 1.61-1.50 (m, 3H), 1.42-1.39 (m, 2H), 1.27 (d, J=3.2 Hz, 1H).

MH+ 386.

### Example 67

### Preparation of (+)-3-Hydroxy-2-(isoquinolin-5-ylphenylamino)morphinan TFA salt

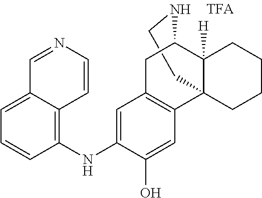

¹H NMR (400 MHz, CD₃OD): δ 9.61 (s, 1H), 8.67 (s, 1H), 8.48 (d, J=6.8 Hz, 1H), 7.86 (d, J=8.0 Hz, 1H), 7.80 (t, J=8.0 Hz, 1H), 7.41 (d, J=7.6 Hz, 1H), 7.02 (s, 1H), 6.95 (s, 1H), 3.65-3.63 (m, 1H), 3.23 (d, J=6.4 Hz, 1H), 3.12 (d, J=12.4 Hz, 1H), 2.89-2.85 (m, 2H), 2.43 (s, 1H), 1.92 (d, J=12.4 Hz, 1H), 1.81 (d, J=13.6 Hz, 1H), 1.72 (s, 1H), 1.64-1.61 (m, 3H), 1.45-1.41 (m, 3H), 1.27 (s, 2H).

MH+ 386.

### Example 68

### Preparation of (+)-3-Hydroxy-2-(quinolin-6-ylphenylamino)morphinan TFA salt

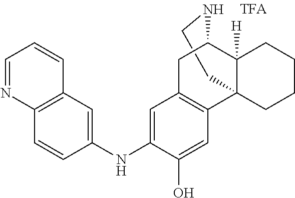

¹H NMR (400 MHz, CD₃OD): δ 8.77-8.72 (m, 2H), 8.02-7.98 (m, 2H), 7.83-7.81 (m, 2H), 7.35 (d, J=3.2 Hz, 1H), 7.21 (d, J=4.8 Hz, 1H), 6.95 (d, J=4.8 Hz, 1H), 3.69 (s, 1H), 3.14 (d, J=12.0 Hz, 2H), 2.90-2.83 (m, 4H), 2.42 (s, 1H), 2.13 (s, 1H), 1.95-1.91 (m, 1H), 1.83-1.75 (m, 3H), 1.64-1.53 (m, 3H), 1.43 (d, J=10.8 Hz, 1H).

MH+ 386.

### Example 69

### Preparation of (+)-3-Hydroxy-2-((1H-indazol-5-yl)amino)morphinan TFA salt

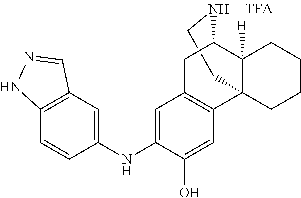

¹H NMR (400 MHz, DMSO-d₆): δ 8.58 (s, 2H), 7.90 (s, 1H), 7.44 (d, J=8.4 Hz, 1H), 7.37 (s, 1H), 7.21 (dd, J=8.8, 1.6 Hz, 1H), 6.85 (s, 1H), 6.75 (s, 1H), 3.97 (s, 1H), 3.58 (s, 1H), 3.06-3.00 (m, 2H), 2.73 (d, J=18.8 Hz, 1H), 2.59-2.56 (m, 1H), 2.26-2.23 (m, 1H), 1.80 (d, J=12.0 Hz, 1H), 1.72-1.62 (m, 2H), 1.51 (d, J=10.4 Hz, 1H), 1.42 (d, J=12.8 Hz, 1H), 1.34-1.26 (m, 3H), 1.00 (q, J=12.8 Hz, 1H).

MH+ 375.

### Example 70

### Preparation of (+)-3-Hydroxy-2-((1H-indazol-5-yl)amino)morphinan TFA salt

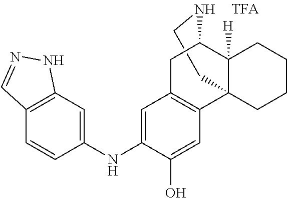

¹H NMR (400 MHz, DMSO-d₆): δ 8.63 (s, 2H), 7.85 (s, 1H), 7.53 (d, J=8.8 Hz, 1H), 7.05 (s, 1H), 6.95 (s, 1H), 6.91 (dd, J=8.8, 2.0 Hz, 1H), 6.81 (s, 1H), 3.96 (s, 1H), 3.62 (s, 1H), 3.07 (dd, J=18.8, 6.0 Hz, 2H), 2.80 (d, J=19.2 Hz, 1H), 2.59-2.56 (m, 1H), 2.33-2.24 (m, 1H), 1.82 (d, J=12.4 Hz, 1H), 1.72-1.66 (m, 2H), 1.53 (d, J=11.2 Hz, 1H), 1.45 (d, J=14.0 Hz, 2H), 1.35-1.21 (m, 3H), 1.05-0.99 (m, 1H).

MH+ 375.

### Example 71

### Preparation of (+)-3-Hydroxy-24(5,6,7,8-tetrahydronaphthalen-2-ybamino)morphinan TFA salt

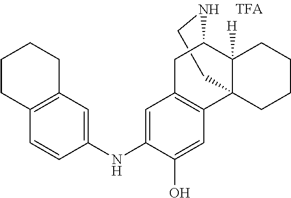

¹H NMR (400 MHz, DMSO-d₆): δ 9.303 (s, 1H), 8.56 (s, 2H), 6.89 (d, J=9.2 Hz, 1H), 6.82 (dd, J=8.4, 2.4 Hz, 1H), 6.75 (d, J=17.2 Hz, 2H), 3.59 (s, 1H), 3.42 (s, 1H), 3.04 (dd, J=19.2, 6.4 Hz, 2H), 2.75 (d, J=18.8 Hz, 1H), 2.64-2.57 (m, 1H), 2.23 (d, J=11.2 Hz, 1H), 1.79 (d, J=12.4 Hz, 1H), 1.66-1.62 (m, 2H), 1.51 (d, J=11.6 Hz, 1H), 1.41 (d, J=12.4 Hz, 2H), 1.33-1.20 (m, 3H), 1.04-0.96 (m, 1H).

MH+ 389.

### Example 72

### Preparation of (+)-3-Hydroxy-2-methylthiomorphinan TFA salt

### Step 1: Preparation of (+)-3-Methoxy-2-methylthio-N-(benzyloxycarbonyl)morphinan

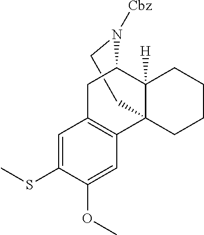

To a solution of (+)-2-iodo-3-methoxy-N-(benzyloxycarbonyl)morphinan obtained in step 3 of Example 17 (533 mg, 1.03 mmol) in EtOH (10 mL) were added NaSMe (86.9 mg, 1.24 mmol), NatBuO (148 mg, 1.55 mmol), and Pd(PPh₃)₄ (119 mg, 0.103 mmol). The resulting reaction mixture was irradiated in a microwave reactor (Biotage) for 30 min at 160° C. After the reaction was completed, water (10 mL) was added thereto. The resulting mixture was extracted with EtOAc (15 mL×2). The combined organic phase was dried over MgSO₄, filtered, and evaporated under vacuum. The residue was purified by flash column chromatography (Biotage SP1™) to provide the title compound (385 mg, 85%) as a white solid.

MH+ 438.

### Step 2: Preparation of (+)-3-Hydroxy-2-methylthiomorphinan TFA salt

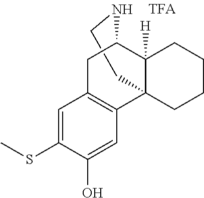

To a solution of (+)-3-methoxy-2-methylthio-N-(benzyloxycarbonyl)morphinan obtained in step 1 (385 mg, 0.880 mmol) in DCM (10 mL) was added BBr₃ solution (1M in DCM, 2.6 mL, 2.60 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (233 mg, 66%) as a white solid.

¹H NMR (400 MHz, CD₃OD): δ 7.03 (s, 1H), 6.77 (s, 1H), 3.65 (dd, J=6.0, 3.2 Hz, 1H), 3.24 (dd, J=19.2, 6.4 Hz, 1H), 3.08 (dd, J=13.2, 3.2 Hz, 1H), 2.90 (d, J=19.2 Hz, 1H), 2.75 (td, J=13.6, 3.6 Hz, 1H), 2.39-2.37 (m, 4H), 1.88 (dt, J=12.8, 3.2 Hz, 1H), 1.78 (td, J=6.0, 4.8 Hz, 1H), 1.70 (d, J=10.8 Hz, 1H), 1.57-1.27 (m, 6H), 1.13-1.09 (m, 1H).

MH+ 290.

The following compound of Example 73 was obtained by repeating the procedure of Example 72.

### Example 73

### Preparation of (+)-3-Hydroxy-2-phenylthiomorphinan TFA salt

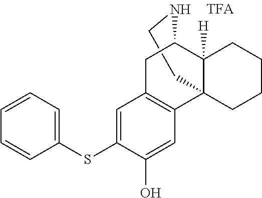

¹H NMR (400 MHz, CD₃OD): δ 7.28-7.16 (m, 5H), 7.05 (s, 1H), 6.90 (s, 1H), 3.63 (dd, J=6.0, 3.2 Hz, 1H), 3.18 (dd, J=19.2, 6.4 Hz, 1H), 3.09 (dd, J=13.2, 3.2 Hz, 1H), 2.84-2.71 (m, 2H), 2.44-2.39 (m, 1H), 1.89 (dt, J=12.4, 3.2 Hz, 1H), 1.80 (td, J=13.6, 4.8 Hz, 1H), 1.73 (d, J=13.2 Hz, 1H), 1.61-1.27 (m, 6H), 1.16-1.06 (m, 1H).

MH+ 352.

### Example 74

### Preparation of (+)-2-(4-Chlorophenol)-3-hydroxymorphinan TFA salt

### Step 1: (+)-2-(4-Chlorophenyl)-3-methoxy-N-(benzyloxycarbonyl)morphinan

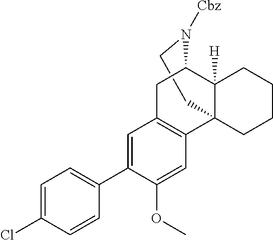

To a solution of (+)-2-iodo-3-methoxy-N-(benzyloxycarbonyl)morphinan obtained in step 3 of Example 17 (533 mg, 1.03 mmol) in 1,4-dioxane (5 mL) were added 4-chlorophenylboronic acid (324 mg, 2.07 mmol), K₂CO₃ (572 mg, 4.14 mmol), and Pd(PPh₃)₄ (119 mg, 0.103 mmol). The resulting reaction mixture was irradiated in a microwave reactor (Biotage) for 30 min at 160° C. After the reaction was completed, water (10 mL) was added thereto. The resulting mixture was extracted with EtOAc (15 mL×2). The combined organic phase was dried over MgSO₄, filtered, and evaporated under vacuum. The residue was purified by flash column chromatography (Biotage SP1™) to provide the title compound (378 mg, 73%) as a yellow solid.

MH+ 503.

### Step 2: Preparation of (+)-2-(4-Chlrophenyl)-3-hydroxymorphinan TFA salt

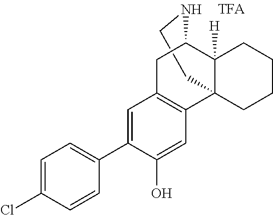

To a solution of (+)-2-(4-chlorophenyl)-3-methoxy-N-(benzyloxycarbonyl)morphinan obtained in step 1 (214 mg, 0.427 mmol) in DCM (10 mL) was added BBr₃ solution (1M in DCM, 120 μL, 1.30 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (152 mg, 76%) as a white solid.

¹H NMR (400 MHz, CD₃OD): δ 7.54 (d, J=8.4 Hz, 2H), 7.35 (d, J=8.4 Hz, 2H), 7.10 (s, 1H), 6.89 (s, 1H), 3.68 (dd, J=5.6, 3.2 Hz, 1H), 3.27 (dd, J=19.2, 6.0 Hz, 1H), 3.11 (dd, J=13.2, 3.2 Hz, 1H), 2.96 (d, J=18.8 Hz, 1H), 2.81 (td, J=13.6, 3.2 Hz, 1H), 2.42 (d, J=13.2 Hz, 1H), 2.00-1.93 (m, 1H), 1.84 (td, J=14.0, 4.4 Hz, 1H), 1.72 (d, J=12.8 Hz, 1H), 1.66-1.13 (m, 6H), 0.94-0.86 (m, 1H). MH+ 354.

The following compounds of Examples 75 to 80 were obtained by repeating the procedure of Example 74.

### Example 75

### Preparation of (+)-3-Hydroxy-2-(4-methylphenyl)morphinan TFA salt

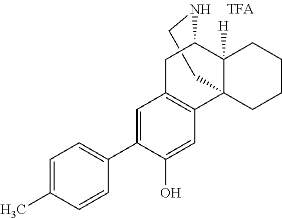

¹H NMR (400 MHz, CD₃OD): δ 7.42 (d, J=8.0 Hz, 2H), 7.18 (d, J=8.0 Hz, 2H), 7.08 (s, 1H), 6.87 (s, 1H), 3.67-3.66 (m, 1H), 3.28-3.25 (m, 1H), 3.11 (dd, J=13.2, 3.2 Hz, 1H), 2.94 (d, J=18.8 Hz, 1H), 2.82 (td, J=13.2, 3.2 Hz, 1H), 2.43 (d, J=15.6 Hz, 1H), 2.34 (s, 3H), 2.00-1.91 (m, 1H), 1.82 (td, J=13.6, 4.8 Hz, 1H), 1.73 (d, J=11.2 Hz, 1H), 1.61-1.14 (m, 6H), 0.94-0.88 (m, 1H).

MH+ 334.

### Example 76

### Preparation of (+)-2-(2,4-Dichlorophenyl)-3-hydroxymorphinan TFA salt

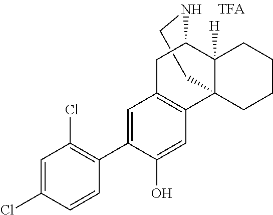

¹H NMR (400 MHz, CD₃OD): δ 7.50 (d, J=1.6 Hz, 1H), 7.35-7.27 (m, 2H), 6.95 (s, 1H), 6.89 (s, 1H), 3.69-3.66 (m, 1H), 3.33-3.24 (m, 1H), 3.13 (dd, J=13.2, 3.2 Hz, 1H), 2.92 (d, J=18.8 Hz, 1H), 2.82 (td, J=13.2, 3.2 Hz, 1H), 2.44 (d, J=13.2 Hz, 1H), 1.93 (d, J=12.4 Hz, 1H), 1.83 (td, J=13.6, 4.4 Hz, 1H), 1.75 (d, J=12.8 Hz, 1H), 1.64-1.14 (m, 6H), 0.95-0.87 (m, 1H).

MH+ 388.

### Example 77

### Preparation of (+)-2-(4-Fluorophenyl)-3-hydroxymorphinan TFA salt

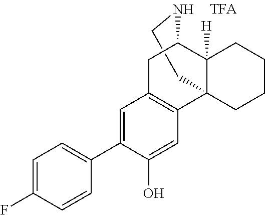

¹H NMR (400 MHz, CD₃OD): δ 7.57-7.54 (m, 2H), 7.11-7.07 (m, 3H), 6.88 (s, 1H), 3.67-3.66 (m, 1H), 3.33-3.26 (m, 1H), 3.09 (dd, J=12.4, 4.0 Hz, 1H), 2.93 (d, J=18.8 Hz, 1H), 2.82 (td, J=13.2, 3.6 Hz, 1H), 2.43 (d, J=12.0 Hz, 1H), 1.91 (d, J=12.0 Hz, 1H), 1.82 (td, J=14.0, 4.8 Hz, 1H), 1.74 (d, J=13.6 Hz, 1H), 1.66-1.16 (m, 6H), 0.95-0.88 (m, 1H).

MH+ 338.

### Example 78

### Preparation of (+)-2-(3-Cyanophenyl)-3-hydroxymorphinan TFA salt

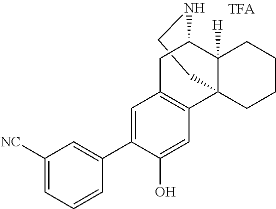

¹H NMR (400 MHz, CD₃OD): δ 7.96 (s, 1H) 7.91 (d, J=8.0 Hz, 1H), 7.65 (d, J=8.0 Hz, 1H), 7.58 (t, J=8.0 Hz, 1H), 7.19 (s, 1H), 6.94 (s, 1H), 3.68-3.64 (m, 1H), 3.36-3.31 (m, 1H), 3.14 (dd, J=13.2, 4.0 Hz, 1H), 2.98 (d, J=19.2 Hz, 1H), 2.84 (td, J=13.6, 3.2 Hz, 1H), 2.46 (d, J=13.6 Hz, 1H), 1.95 (d, J=12.4 Hz, 1H), 1.85 (td, J=13.6, 4.4 Hz, 1H), 1.76 (d, J=12.4 Hz, 1H), 1.65-1.29 (m, 6H), 1.23-1.17 (m, 1H).

MH+ 345.

### Example 79

### Preparation of (+)-3-Hydroxy-2-(4-trifluorophenyl)morphinan TFA salt

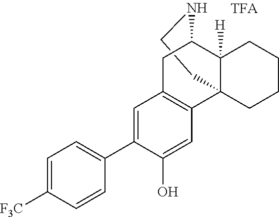

¹H NMR (400 MHz, CD₃OD): δ 8.04 (d, J=8.4 Hz, 2H), 7.71 (d, J=8.4 Hz, 1H), 7.19, (s, 1H), 6.93 (s, 1H), 3.74-3.70 (m, 1H), 3.36-3.30 (m, 1H), 3.14 (dd, J=13.2, 3.6 Hz, 1H), 2.98 (d, J=18.8 Hz, 1H), 2.84 (td, J=13.2, 3.2 Hz, 1H), 2.46 (d, J=12.8 Hz, 1H), 1.95 (d, J=12.4 Hz, 1H), 1.85 (td, J=14.0, 4.4 Hz, 1H), 1.75 (d, J=13.2 Hz, 1H), 1.65-1.40 (m, 6H), 1.20-1.17 (m, 1H).

MH+ 388.

### Example 80

### (+)-3-Hydroxy-2-phenylmorphinan TFA salt

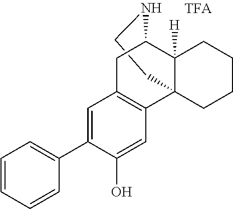

¹H NMR (400 MHz, CD₃OD): δ 7.54 (dd, J=8.4, 1.2 Hz, 2H), 7.36 (t, J=7.6 Hz, 2H), 7.26 (td, J=8.4, 1.2 Hz, 1H), 7.10, (s, 1H), 6.88 (s, 1H), 3.68-3.67 (m, 1H), 3.33-3.23 (m, 1H), 3.11 (dd, J=12.8, 3.2 Hz, 1H), 2.96 (d, J=19.2 Hz, 1H), 2.83 (td, J=13.6, 3.2 Hz, 1H), 2.44 (d, J=13.2 Hz, 1H), 1.94 (d, J=12.4 Hz, 1H), 1.84 (td, J=13.6, 4.8 Hz, 1H), 1.74 (d, J=12.0 Hz, 1H), 1.62-1.14 (m, 6H), 0.88-0.86 (m, 1H).

MH+ 320.

### Example 81

### Preparation of (+)-3-Hydroxy-2-isobutylmorphinan TFA salt

### Step 1: Preparation of (+)-2-Isobutyl-3-methoxy-N-(benzyloxycarbonyl)morphinan

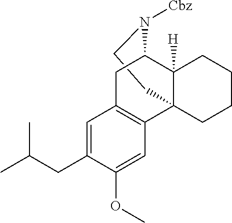

To a solution of (+)-2-iodo-3-methoxy-N-(benzyloxycarbonyl)morphinan obtained in step 3 of Example 17 (1.07 g, 2.07 mmol) in 1,4-dioxane (10 mL) were added isobutylboronic acid (211 mg, 2.07 mmol), Cs₂CO₃ (2.70 g, 8.28 mmol), and (dppf)PdCl₂CH₂Cl₂ (169 mg, 0.207 mmol). The resulting reaction mixture was irradiated in a microwave reactor (Biotage) for 30 min at 160° C. After the reaction was completed, water (10 mL) was added thereto. The resulting mixture was extracted with EtOAc (15 mL×2). The combined organic phase was dried over MgSO₄, filtered, and evaporated under vacuum. The residue was purified by flash column chromatography (Biotage SP1™) to provide the title compound (207 mg, 22%) as a white solid.

MH+ 448.

### Step 2: Preparation of (+)-3-Hydroxy-2-isobutylmorphinan TFA salt

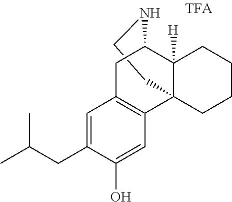

To a solution of (+)-2-Isobutyl-3-methoxy-N-(benzyloxycarbonyl)morphinan (40) (346 mg, 0.774 mmol) in DCM (10 mL) was added BBr₃ solution (1M in DCM, 2.3 mL, 2.30 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (91.0 mg, 28%) as a white solid.

¹H NMR (400 MHz, CD₃OD): δ 6.84 (s, 1H), 6.72 (s, 1H), 3.65-3.63 (m, 1H), 3.21 (d, J=18.8 Hz, 1H), 3.07 (d, J=10.8 Hz, 1H), 2.88 (d, J=18.8 Hz, 1H), 2.76-2.72 (m, 1H), 2.42-2.37 (m, 3H), 1.94-1.88 (m, 2H), 1.78 (t, J=12.8 Hz, 1H), 1.70 (d, J=12.0 Hz, 1H), 1.55-1.11 (m, 6H), 0.94-0.91 (m, 1H), 0.88 (d, J=6.4 Hz, 6H).

MH+ 300.

The following compounds of Examples 82 to 85 were obtained by repeating the procedure of Example 81.

### Example 82

### Preparation of (+)-3-Hydroxy-2-propylmorphinan TFA salt

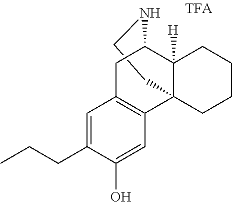

¹H NMR (400 MHz, CD₃OD): δ 6.87 (s, 1H), 6.72 (s, 1H), 3.65-3.63 (m, 1H), 3.20 (d, J=18.8 Hz, 1H), 3.07 (d, J=11.2 Hz, 1H), 2.87 (d, J=18.4 Hz, 1H), 2.75 (t, J=11.6 Hz, 1H), 2.51 (t, J=7.2 Hz, 2H), 2.38 (d, J=11.6 Hz, 1H), 1.88 (d, J=12.0 Hz, 1H), 1.77-1.68 (m, 2H), 1.61-1.27 (m, 7H), 1.20-1.08 (m, 1H), 0.95-0.90 (m, 4H).

MH+ 286.

### Example 83

### Preparation of (+)-2-Butyl-3-hydroxymorphinan TFA salt

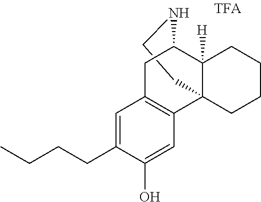

¹H NMR (400 MHz, CD₃OD): δ 6.87 (s, 1H), 6.71 (s, 1H), 3.65-3.63 (m, 1H), 3.20 (d, J=18.0 Hz, 1H), 3.06 (d, J=11.2 Hz, 1H), 2.87 (d, J=18.8 Hz, 1H), 2.74 (t, J=11.6 Hz, 1H), 2.53 (t, J=7.6 Hz, 2H), 2.37 (d, J=12.0 Hz, 1H), 1.89 (d, J=11.6 Hz, 1H), 1.81-1.74 (m, 1H), 1.69 (d, J=11.2 Hz, 1H), 1.56-1.13 (m, 10H), 0.92 (t, J=7.6 Hz, 3H), 0.89-0.87 (m, 1H).

MH+ 300.

### Example 84

### Preparation of (+)-2-Ethyl-3-hydroxymorphinan TFA salt

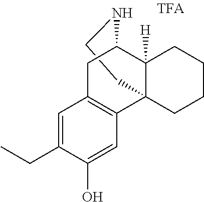

¹H NMR (400 MHz, CD₃OD): δ 6.83 (s, 1H), 6.68 (s, 1H), 3.56-3.52 (m, 1H), 3.03-2.83 (m, 3H), 2.19 (d, J=13.2 Hz, 1H), 1.83 (d, J=12.4 Hz, 1H), 1.67 (t, J=11.4 Hz, 1H), 1.57 (d, J=11.6 Hz, 1H), 1.45 (d, J=11.6 Hz, 1H), 1.39-1.14 (m, 7H), 1.09 (t, J=7.6 Hz, 3H), 0.94-0.82 (m, 2H).

MH+ 272.

### Example 85

### Preparation of (+)-3-Hydroxy-2-methylmorphinan TFA salt

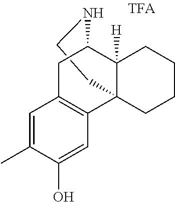

¹H NMR (400 MHz, CD₃OD): δ 6.90 (s, 1H), 6.72 (s, 1H), 3.65-3.61 (m, 1H), 3.24-3.18 (m, 1H), 3.07 (d, J=12.8 Hz, 1H), 2.85 (d, J=18.8 Hz, 1H), 2.78-2.74 (m, 1H), 2.39 (d, J=12.0 Hz, 1H), 2.15 (s, 3H), 1.87 (d, J=12.0 Hz, 1H), 1.77-1.70 (m, 2H), 1.55-1.15 (m, 7H).

MH+ 258.

### Example 86

### Preparation of (+)-3-Hydroxy-2-morpholinomorphinan TFA salt

### Step 1: Preparation of (+)-3-Hydroxy-2-nitro-N-(benzyloxycarbonyl)morphinan

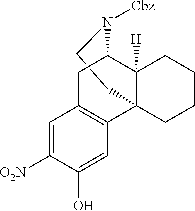

To a solution of (+)-3-hydroxymorphinan (20.7 g, 85.1 mmol) in formic acid (200 mL) was added HNO₃ (70%, 5.5 mL, 85.1 mmol) at 0° C. The resulting reaction mixture was stirred vigorously at r.t. overnight and evaporated under vacuum. The residue was neutralized by saturated NaHCO₃ solution and extracted by EtOAc (200 mL×2). The combined organic phase was dried over MgSO₄, filtered, and evaporated under vacuum. To the residue was added 1,4-dioxane (170 mL) 1N NaOH (170 mL). To the resulting solution was added Cbz-Cl (12.2 mL, 85.1 mmol) at 0° C. and then the reaction mixture was stirred at r.t. overnight. After the reaction was completed, water (200 mL) was added thereto. The mixture thus obtained was extracted with diethyl ether (500 mL×2). The combined organic phase was dried over MgSO₄, filtered, and evaporated under vacuum. The residue was purified by flash column chromatography (Biotage SP1™) to provide the title compound (21.0 g, 58%) as a yellow solid.

¹H NMR (400 MHz, CDCl₃): δ 10.36 (s, 1H), 7.85 (s, 1H), 7.38-7.26 (m, 5H), 7.10 (s, 1H), 5.21-5.10 (m, 2H), 4.42 (d, J=46.8 Hz, 1H), 4.00-3.88 (m, 1H), 3.12 (td, J=13.2, 5.2 Hz, 1H), 2.79-2.71 (m, 1H), 2.67-2.54 (m, 1H), 2.36 (d, J=13.6 Hz, 1H), 1.77-1.50 (m, 5H), 1.45-1.17 (m, 4H), 1.02-0.93 (m, 1H).

MH+ 423.

### Step 2: Preparation of (+)-3-Methoxy-2-nitro-N-(benzyloxycarbonyl)morphinan

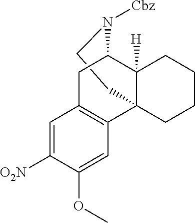

To (+)-3-hydroxy-2-nitro-N-(benzyloxycarbonyl)morphinan obtained in step 1 (21.0 g, 49.7 mmol) and K₂CO₃ (13.7 g, 99.4 mmol) in acetone (250 mL) was added iodomethane (4.65 mL, 74.6 mmol) at r.t. The reaction mixture was stirred at r.t. overnight. After the reaction was completed, water (300 mL) was added thereto. The resulting mixture was extracted with EtOAc (300 mL×2). The combined organic phase was dried over MgSO₄, filtered, and evaporated under vacuum. The residue was purified by flash column chromatography (Biotage SP1™) to provide the title compound (21.2 g, 98%) as a yellow solid.

MH+ 437.

### Step 3: Preparation of (+)-2-Amino-3-methoxy-N-(benzyloxycarbonyl)morphinan

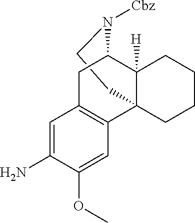

To a solution of (+)-3-methoxy-2-nitro-N-(benzyloxycarbonyl)morphinan obtained in step 2 (21.2 g, 48.6 mmol) and hydrazine hydrate (11.8 mL, 243 mmol) in MeOH (100 mL) was added Raney Ni (slurry in water, 1 mL) at r.t. The resulting reaction mixture was stirred at r.t. for 2 hr. After the reaction was completed, the Raney Ni was separated by filtration over celite and the solvent evaporated under vacuum. The residue was purified by flash column chromatography (Biotage SP1™) to provide the title compound (16.6 g, 81%) as a yellow solid.

¹H NMR (400 MHz, CDCl₃): δ 7.37-7.26 (m, 5H), 6.64 (s, 1H), 6.44 (d, J=10.8 Hz, 1H), 5.20-5.12 (m, 2H), 4.32 (d, J=40.0 Hz, 1H), 3.94-3.82 (m, 4H), 3.01 (td, J=17.6, 5.6 Hz, 1H), 2.76-2.64 (m, 1H), 2.60-2.52 (m, 1H), 2.30 (d, J=9.6 Hz, 1H), 1.64-1.24 (m, 9H), 1.12-1.09 (m, 1H).

MH+ 407.

### Step 4: Preparation of (+)-3-Methoxy-2-morpholino-N-(benzyloxycarbonyl)morphinan

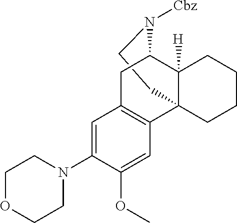

To a solution of (+)-2-amino-3-methoxy-N-(benzyloxycarbonyl)morphinan obtained in step 3 (1.00 g, 2.46 mmol) and NaHCO₃ (454 mg, 5.41 mmol) in DMF (20 mL) was added 2-chloroethyl ether (320 μL, 2.71 mmol) at r.t. The resulting reaction mixture was stirred at 100° C. overnight. After the reaction was completed, water (40 mL) was added thereto. The mixture thus obtained was extracted with EtOAc (50 mL×2). The combined organic phase was dried over MgSO₄, filtered, and evaporated under vacuum. The residue was purified by flash column chromatography (Biotage SP1™) to provide the title compound (1.06 g, 90%) as a white solid.

MH+ 477.

### Step 5: Preparation of (+)-3-Hydroxy-2-morpholinomorphinan TFA salt

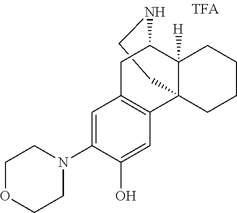

To a solution of (+)-3-methoxy-2-morpholino-N-(benzyloxycarbonyl)morphinan obtained in step 4 (1.06 g, 2.22 mmol) in DCM (10 mL) was added BBr₃ solution (1M in DCM, 6.7 mL, 6.70 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (197 mg, 20%) as a brown solid.

¹H NMR (400 MHz, CD₃OD): δ 7.27 (s, 1H), 6.97 (s, 1H), 4.10-3.94 (m, 4H), 3.71-3.69 (m, 1H), 3.52 (t, J=4.8 Hz, 4H), 3.34-3.24 (m, 1H), 3.11 (dd, J=13.2, 3.6 Hz, 1H), 2.98 (d, J=19.2 Hz, 1H), 2.73 (td, J=13.6, 3.6 Hz, 1H), 2.39 (d, J=14.0 Hz, 1H), 1.95-1.91 (m, 1H), 1.83 (td, J=13.6, 4.8 Hz, 1H), 1.70 (d, J=12.4 Hz, 1H), 1.60-1.38 (m, 5H), 1.29-1.20 (m, 1H), 1.11-1.01 (m, 1H).

MH+ 329.

### Example 87

### Preparation of (+)-3-Hydroxy-2-isopropylaminomorphinan TFA salt

### Step 1: Preparation of (+)-2-Isopropylamino-3-methoxy-N-(benzyloxycarbonyl)morphinan

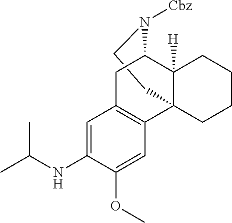

To a solution of (+)-2-amino-3-methoxy-N-(benzyloxycarbonyl)morphinan obtained in step 3 of Example 86 (1.00 g, 2.46 mmol) in 1,2-dichloroethane (20 mL) was added acetone (540 μL, 7.38 mmol) at r.t. After stirring for 10 min at r.t., NaBH(OAc)₃ (1.56 g, 7.38 mmol) was added thereto. The resulting mixture was stirred overnight and washed with saturated NaHCO₃. The combined water layer was extracted EtOAc (50 mL×2). The combined organic phase was dried over MgSO₄, filtered, and evaporated under vacuum. The residue was purified by flash column chromatography (Biotage SP1™) to provide the title compound (843 mg, 76%) as a white solid.

MH+ 449.

### Step 2: Preparation of (+)-3-Hydroxy-2-isopropylaminomorphinan TFA salt

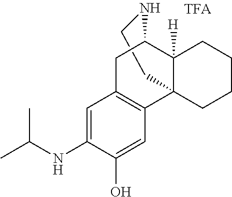

To a solution of (+)-2-isopropylamino-3-methoxy-N-(benzyloxycarbonyl)morphinan obtained in step 1 (843 mg, 1.88 mmol) in DCM (10 mL) was added BBr₃ solution (1M in DCM, 5.64 mL, 5.64 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (508 mg, 65%) as a colorless gum.

¹H NMR (400 MHz, CD₃OD): δ 7.24 (s, 1H), 7.04 (s, 1H), 3.84-3.81 (m, 1H), 3.75-3.73 (m, 1H), 3.33-3.32 (m, 1H), 3.14 (dd, J=13.6, 3.6 Hz, 1H), 3.06 (d, J=19.2 Hz, 1H), 2.73 (td, J=13.6, 3.6 Hz, 1H), 2.41 (d, J=13.6 Hz, 1H), 2.00 (dt, J=12.8, 3.2 Hz, 1H), 1.88 (td, J=14.0, 4.8 Hz, 1H), 1.71 (d, J=12.4 Hz, 1H), 1.62-1.40 (m, 5H), 1.37 (d, J=6.8 Hz, 6H), 1.31-1.23 (m, 1H), 1.08-1.03 (m, 1H).

MH+ 301.

The following compounds of Examples 88 to 101 were obtained by repeating the procedure of Example 87.

### Example 88

### Preparation of (+)-3-Hydroxy-2-propylaminomorphinan TFA salt

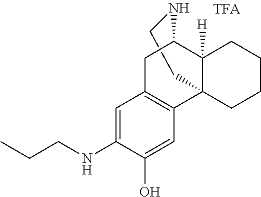

¹H NMR (400 MHz, CD₃OD): δ 7.16 (s, 1H), 7.00 (s, 1H), 3.71-3.68 (m, 1H), 3.32-3.25 (m, 3H), 3.12 (dd, J=13.6, 3.6 Hz, 1H), 2.96 (d, J=19.2 Hz, 1H), 2.72 (td, J=13.6, 3.6 Hz, 1H), 2.39 (d, J=13.6 Hz, 1H), 1.94 (dt, J=12.8, 3.2 Hz, 1H), 1.87-1.69 (m, 4H), 1.60-1.39 (m, 5H), 1.29-1.23 (m, 1H), 1.11-1.07 (m, 1H), 1.03 (t, J=7.2 Hz, 3H).

MH+ 301.

### Example 89

### Preparation of (+)-2-(Heptan-4-ylamino)-3-hydroxymorphinan TFA salt

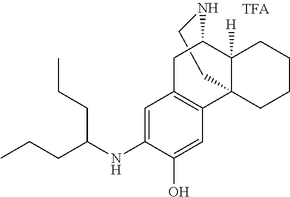

¹H NMR (400 MHz, CD₃OD): δ 7.17 (s, 1H), 7.03 (s, 1H), 3.71 (dd, J=6.0, 3.2 Hz, 1H), 3.60-3.57 (m, 1H), 3.32-3.25 (m, 1H), 3.13 (dd, J=13.2, 3.2 Hz, 1H), 2.98 (d, J=19.6 Hz, 1H), 2.70 (td, J=13.6, 3.6 Hz, 1H), 2.40 (d, J=14.0 Hz, 1H), 1.96 (dt, J=12.4, 2.8 Hz, 1H), 1.84 (td, J=13.6, 4.8 Hz, 1H), 1.72-1.35 (m, 14H), 1.25-1.22 (m, 1H), 1.09-1.03 (m, 1H), 0.92 (t, J=7.2 Hz, 6H).

MH+ 357.

### Example 90

### Preparation of (+)-2-Butylamino-3-hydroxymorphinan TFA salt

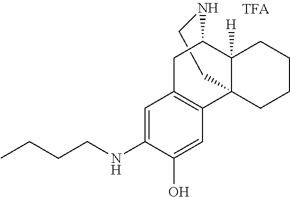

¹H NMR (400 MHz, CD₃OD): δ 7.20 (s, 1H), 7.01 (s, 1H), 3.70 (dd, J=6.0, 3.2 Hz, 1H), 3.36-3.24 (m, 3H), 3.12 (dd, J=13.6, 3.6 Hz, 1H), 2.97 (d, J=19.2 Hz, 1H), 2.71 (td, J=13.6, 3.6 Hz, 1H), 2.39 (d, J=14.0 Hz, 1H), 1.95 (dt, J=12.4, 3.2 Hz, 1H), 1.84 (td, J=14.0, 4.8 Hz, 1H), 1.76-1.68 (m, 3H), 1.61-1.39 (m, 7H), 1.29-1.22 (m, 1H), 1.10-1.00 (m, 1H), 0.97 (t, J=7.2 Hz, 3H).

MH+ 315.

### Example 91

### Preparation of (+)-3-Hydroxy-2-(1-phenylethylamino)morphinan TFA salt

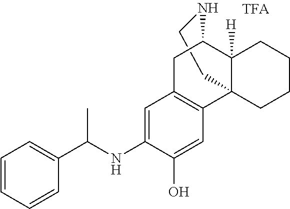

¹H NMR (400 MHz, CD₃OD): δ 7.36-7.32 (m, 5H) 6.94 (s, 1H), 6.65 (d, J=14.8 Hz, 1H), 4.81-4.80 (m, 1H), 3.62 (dd, J=5.6, 3.2 Hz, 1H), 3.11-2.96 (m, 2H), 2.80 (d, J=19.2 Hz, 1H), 2.68-2.63 (m, 1H), 2.35 (d, J=14.0 Hz, 1H), 1.89 (d, J=12.0 Hz, 1H), 1.83-1.67 (m, 5H), 1.59-1.37 (m, 5H), 1.22-1.16 (m, 1H), 1.04-0.84 (m, 1H).

MH+ 363.

### Example 92

### (+)-2-Cyclopentylamino-3-hydroxymorphinan TFA salt

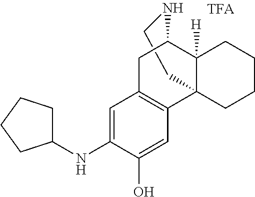

¹H NMR (400 MHz, CD₃OD): δ 7.17 (s, 1H), 7.01 (s, 1H), 4.05-3.99 (m, 1H), 3.70 (dd, J=6.0, 3.2 Hz, 1H), 3.33-3.25 (m, 1H), 3.12 (dd, J=13.2, 3.6 Hz, 1H), 2.97 (d, J=19.2 Hz, 1H), 2.71 (td, J=13.6, 3.6 Hz, 1H), 2.39 (d, J=14.0 Hz, 1H), 2.00-1.38 (m, 16H), 1.29-1.09 (m, 1H), 1.07-1.00 (m, 1H).

MH+ 327.

### Example 93

### Preparation of (+)-2-Cyclohexylamino-3-hydroxymorphinan TFA salt

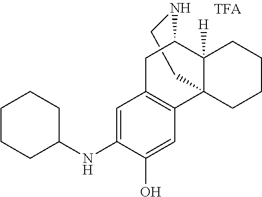

¹H NMR (400 MHz, CD₃OD): δ 7.13 (s, 1H), 7.02 (s, 1H), 3.70 (dd, J=5.6, 3.2 Hz, 1H), 3.49-3.41 (m, 1H), 3.34-3.23 (m, 1H), 3.12 (dd, J=13.2, 3.6 Hz, 1H), 2.96 (d, J=19.2 Hz, 1H), 2.72 (td, J=13.6, 3.6 Hz, 1H), 2.40 (d, J=14.4 Hz, 1H), 2.04-1.20 (m, 19H), 1.08-1.04 (m, 1H).

MH+ 341.

### Example 94

### Preparation of (+)-2-Cycloheptylamino-3-hydroxymorphinan TFA salt

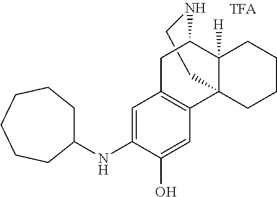

¹H NMR (400 MHz, CD₃OD) 7.16 (s, 1H), 7.02 (s, 1H), 3.71-3.62 (m, 2H), 3.33-3.25 (m, 1H), 3.12 (dd, J=13.6, 3.6 Hz, 1H), 2.97 (d, J=19.6 Hz, 1H), 2.71 (td, J=13.2, 3.6 Hz, 1H), 2.39 (d, J=14.0 Hz, 1H), 2.08-2.03 (m, 2H), 1.96-1.93 (m, 1H), 1.88-1.39 (m, 17H), 1.29-1.20 (m, 1H), 1.11-1.00 (m, 1H).

MH+ 355.

### Example 95

### Preparation of (+)-2-(sec-Butylamino)-3-hydroxymorphinan TFA salt

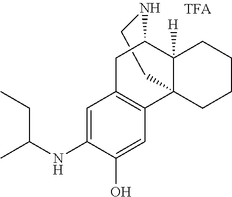

¹H NMR (400 MHz, CD₃OD): δ 7.16 (s, 1H), 7.03 (s, 1H), 3.70 (dd, J=6.0, 3.2 Hz, 1H), 3.63-3.55 (m, 1H), 3.33-3.25 (m, 1H), 3.16 (dd, J=13.2, 3.6 Hz, 1H), 2.97 (d, J=19.2 Hz, 1H), 2.71 (td, J=13.6, 3.6 Hz, 1H), 2.40 (d, J=14.0 Hz, 1H), 1.95 (d, J=14.0 Hz, 1H), 1.89-1.80 (m, 2H), 1.71 (d, J=12.8 Hz, 1H), 1.66-1.39 (m, 6H), 1.31 (d, J=6.8 Hz, 3H), 1.26-1.23 (m, 1H), 1.11-1.07 (m, 1H), 1.01 (td, J=7.6, 1.2 Hz, 3H).

MH+ 315.

### Example 96

### Preparation of (+)-2-(Dipropylamino)-3-hydroxymorphinan TFA salt

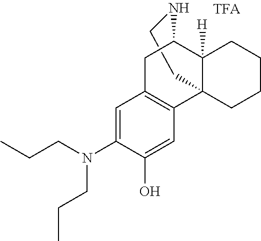

¹H NMR (400 MHz, CD₃OD): δ 7.40 (s, 1H), 7.04 (s, 1H), 3.72 (dd, J=6.0, 3.2 Hz, 1H), 3.57-3.46 (m, 4H), 3.35-3.28 (m, 1H), 3.14 (dd, J=13.6, 3.2 Hz, 1H), 3.01 (d, J=19.2 Hz, 1H), 2.71 (td, J=13.6, 3.6 Hz, 1H), 2.40 (d, J=14.0 Hz, 1H), 1.96 (dt, J=12.4, 2.8 Hz, 1H), 1.85 (td, J=14.0, 4.8 Hz, 1H), 1.72 (d, J=12.8 Hz, 1H), 1.62-1.40 (m, 9H), 1.29-1.22 (m, 1H), 1.10-1.01 (m, 1H), 0.91 (t, J=7.2 Hz, 6H).

MH+ 343.

### Example 97

### Preparation of (+)-3-Hydroxy-2-(3-trifluoropropylamino)morphinan TFA salt

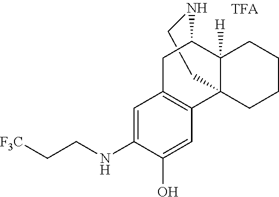

¹H NMR (400 MHz, CD₃OD): δ 6.79 (s, 1H), 6.70 (s, 1H), 3.64 (dd, J=6.0, 3.2 Hz, 1H), 3.51-3.47 (m, 2H), 3.24 (dd, J=19.2, 6.0 Hz, 1H), 3.08 (dd, J=13.2, 3.6 Hz, 1H), 2.88 (d, J=19.2 Hz, 1H), 2.77 (td, J=13.6, 4.0 Hz, 1H), 2.61-2.53 (m, 2H), 2.35 (d, J=13.2 Hz, 1H), 1.88 (dt, J=12.4, 2.8 Hz, 1H), 1.81-1.69 (m, 2H), 1.57-1.24 (m, 6H), 1.18-1.08 (m, 1H).

MH+ 355.

### Example 98

### Preparation of (+)-2-(Dimethylamino)-3-hydroxymorphinan TFA salt

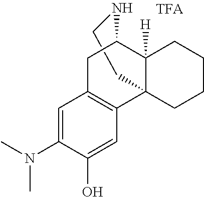

¹H NMR (400 MHz, CD₃OD): δ 7.54 (s, 1H), 7.03 (s, 1H), 3.74 (dd, J=6.0, 3.2 Hz, 1H), 3.35-3.27 (m, 7H), 3.13 (dd, J=13.6, 3.6 Hz, 1H), 3.06 (d, J=19.2 Hz, 1H), 2.73 (td, J=13.6, 3.6 Hz, 1H), 2.40 (d, J=14.4 Hz, 1H), 1.98 (dt, J=12.4, 3.2 Hz, 1H), 1.87 (td, J=14.0, 4.8 Hz, 1H), 1.70 (d, J=12.8 Hz, 1H), 1.61-1.39 (m, 5H), 1.26-1.21 (m, 1H), 1.09-0.99 (m, 1H).

MH+ 287.

### Example 99

### Preparation of (+)-2-(2-Ethoxyethylamino)-3-hydroxymorphinan TFA salt

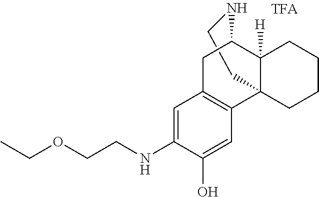

¹H NMR (400 MHz, CD₃OD): δ 7.56 (s, 1H), 6.80 (s, 1H), 3.18 (q, J=7.2 Hz, 2H), 3.64 (dd, J=6.0, 3.6 Hz, 1H), 3.31-3.28 (m, 4H), 3.23 (dd, J=19.2, 6.0 Hz, 1H), 3.07 (dd, J=13.6, 3.6 Hz, 1H), 2.88 (d, J=18.8 Hz, 1H), 2.77 (td, J=13.6, 4.0 Hz, 1H), 2.36 (d, J=13.2 Hz, 1H), 1.88 (dt, J=12.4, 2.8 Hz, 1H), 1.77 (td, J=13.6, 4.8 Hz, 1H), 1.70 (d, J=11.2 Hz, 1H), 1.57-1.35 (m, 6H), 1.29 (t, J=7.2 Hz, 3H), 1.15-1.11 (m, 1H).

MH+ 331.

### Example 100

### Preparation of (+)-3-Hydroxy-2-(2-hydroxyethylamino)morphinan TFA salt

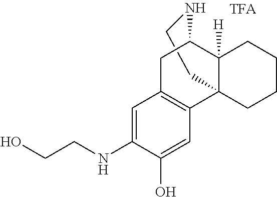

¹H NMR (400 MHz, CD₃OD): δ 7.07 (s, 1H), 6.94 (s, 1H), 3.78-3.75 (m, 2H), 3.68 (dd, J=6.0, 3.2 Hz, 1H), 3.42-3.40 (m, 2H), 3.32 (dd, J=12.4, 1.6 Hz, 1H), 3.11 (dd, J=13.2, 3.2 Hz, 1H), 2.93 (d, J=18.8 Hz, 1H), 2.73 (td, J=13.2, 3.6 Hz, 1H), 2.38 (d, J=13.6 Hz, 1H), 1.92 (dt, J=12.4, 2.8 Hz, 1H), 1.81 (td, J=13.6, 4.8 Hz, 1H), 1.71 (d, J=13.2 Hz, 1H), 1.58-1.39 (m, 5H), 1.31-1.21 (m, 1H), 1.13-1.03 (m, 1H).

MH+ 303.

### Example 101

### Preparation of (+)-2-(Diethylamino)-3-hydroxymorphinan TFA salt

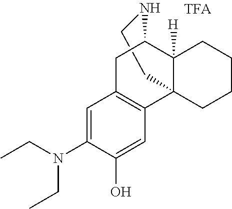

¹H NMR (400 MHz, CD₃OD): δ 7.48 (s, 1H), 7.05 (s, 1H), 3.75 (dd, J=6.0, 3.2 Hz, 1H), 3.71-3.65 (m, 2H), 3.61-3.56 (m, 2H), 3.36-3.29 (m, 1H), 3.15 (dd, J=13.6, 3.6 Hz, 1H), 3.01 (d, J=19.2 Hz, 1H), 2.73 (td, J=13.6, 3.6 Hz, 1H), 2.41 (d, J=14.4 Hz, 1H), 2.00 (dt, J=12.4, 2.8 Hz, 1H), 1.88 (td, J=14.0, 4.8 Hz, 1H), 1.72 (d, J=12.4 Hz, 1H), 1.63-1.41 (m, 5H), 1.27-1.22 (m, 1H), 1.13 (t, J=7.2 Hz, 6H), 1.08-1.01 (m, 1H).

MH+ 315.

### Example 102

### Preparation of (+)-3-Hydroxy-2-(methylpropylamino)morphinan TFA salt

### Step 1: Preparation of (+)-2-(tert-Butyloxycarbonylamino)-3-methoxy-N-(benzyloxycarbonyl)morphinan

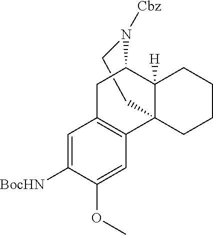

To a solution of (+)-2-amino-3-methoxy-N-(benzyloxycarbonyl)morphinan obtained in step 3 of Example 86 (2.88 g, 6.60 mmol) in THF (40 mL) was added di-tert-butyl dicarbonate (2.16 g, 9.90 mmol) at r.t. The resulting reaction mixture was stirred overnight and then saturated NaHCO₃ (50 mL) was added. The mixture thus obtained was extracted with EtOAc (50 mL×2). The combined organic phase was dried over MgSO₄, filtered, and evaporated under vacuum. The residue was purified by flash column chromatography (Biotage SP1™) to provide the title compound (2.04 g, 61%) as a white solid.

MH+ 507.

### Step 2: Preparation of (+)-2-(tert-Buyloxycarbonyl(methyl)amino)-3-methoxy-N-(benzyloxycarbonyl)morphinan

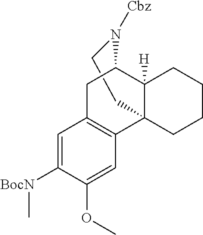

To a solution of (+)-2-(tert-buyloxycarbonylamino)-3-methoxy-N-(benzyloxycarbonyl)morphinan obtained in step 1 (1.00 g, 1.97 mmol) and iodomethane (180 μL, 2.96 mmol) in THF (20 mL) was added NaH (118 mg, 2.96 mmol) at 0° C. The resulting reaction mixture was stirred at r.t. overnight. After the reaction was completed, water (30 mL) was added thereto. The resulting mixture was extracted with EtOAc (30 mL×2). The combined organic phase was dried over MgSO₄, filtered, and evaporated under vacuum. The residue was purified by flash column chromatography (Biotage SP1™) to provide the title compound (847 mg, 83%) as a white solid.

MNa+ 543.

### Step 3: Preparation of (+)-3-Methoxy-2-(methylpropylamino)-N-(benzyloxycarbonyl)morphinan

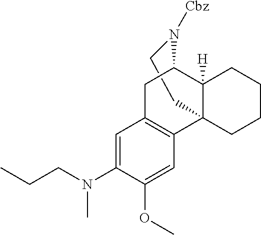

To a solution of (+)-2-(tert-buyloxycarbonyl(methyl)amino)-3-methoxy-N-(benzyloxycarbonyl)morphina n obtained in step 2 (500 mg, 1.19 mmol) in DCM (10 mL) was added TFA (280 μL, 3.57 mmol) at r.t. The resulting reaction mixture was stirred at r.t. for 2 hr. After the reaction was completed, the resulting mixture was evaporated under vacuum. To the residue were added 1,2-dichloroethane (15 mL) and propionaldehyde (170 μL, 2.38 mmol). After stirring for 10 min at r.t., NaBH(OAc)₃ (504 mg, 2.38 mmol) was added to the resulting reaction mixture. The mixture thus obtained was stirred overnight and washed with saturated NaHCO₃. The combined water layer was extracted EtOAc (20 mL×2). The combined organics were dried over MgSO₄, filtered, and evaporated under vacuum. The residue was purified by flash column chromatography (Biotage SP1™) to provide the title compound (540 mg, 98%) as a yellow solid.

MH+ 463.

### Step 4: Preparation of (+)-3-Hydroxy-2-(methylpropylamino)morphinan TFA salt

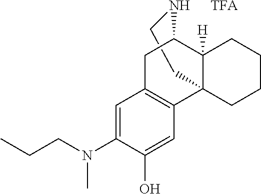

To a solution of (+)-3-Methoxy-2-(methylpropylamino)-N-(benzyloxycarbonyl)morphinan obtained in step 3 (540 mg, 1.17 mmol) in DCM (10 mL) was added BBr₃ solution (1M in DCM, 3.51 mL, 3.51 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (177 mg, 35%) as a yellow solid.

¹H NMR (400 MHz, CD₃OD): δ 7.43 (s, 1H), 7.04 (s, 1H), 3.73-3.70 (m, 1H), 3.54-3.50 (m, 2H), 3.35-3.29 (m, 1H), 3.23 (s, 3H), 3.14 (dd, J=13.6, 3.6 Hz, 1H), 3.01 (d, J=19.2 Hz, 1H), 2.71 (td, J=13.6, 3.6 Hz, 1H), 2.40 (d, J=13.6 Hz, 1H), 1.95 (dt, J=12.8, 3.2 Hz, 1H), 1.84 (td, J=14.0, 4.8 Hz, 1H), 1.72 (d, J=13.2 Hz, 1H), 1.61-1.40 (m, 7H), 1.29-1.21 (m, 1H), 1.10-1.00 (m, 1H), 0.94 (t, J=7.2 Hz, 3H).

MH+ 315.

The following compound of Example 103 was obtained by repeating the procedure of Example 102.

### Example 103

### Preparation of (+)-2-(Ethylmethylamino)-3-hydroxymorphinan TFA salt

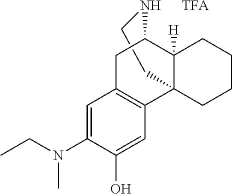

¹H NMR (400 MHz, CD₃OD): δ 7.42 (s, 1H), 7.04 (s, 1H), 3.72 (dd, J=6.0, 3.2 Hz, 1H), 3.63-3.61 (m, 2H), 3.32 (dd, J=6.4, 1.6 Hz, 1H), 3.23 (s, 3H), 3.13 (dd, J=14.4, 3.6 Hz, 1H), 3.00 (d, J=19.2 Hz, 1H), 2.72 (td, J=13.6, 4.0 Hz, 1H), 2.40 (d, J=13.6 Hz, 1H), 1.95 (dt, J=12.8, 3.2 Hz, 1H), 1.85 (td, J=14.0, 4.8 Hz, 1H), 1.71 (d, J=12.4 Hz, 1H), 1.61-1.40 (m, 5H), 1.29-1.21 (m, 1H), 1.18 (t, J=7.2 Hz, 3H), 1.10-0.99 (m, 1H).

MH+ 301.

### Example 104

### Preparation of (+)-2-tert-Butyl-3-hydroxymorphinan TFA salt

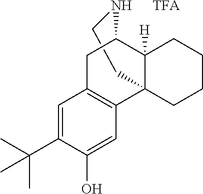

To a solution of (+)-3-hydroxymorphinan (HM) HBr (200 mg, 0.617 mmol) in tert-BuOH (4 mL) was added conc. H₂SO₄ (1 mL, 18.8 mmol) at r.t. The resulting reaction mixture was stirred at 45° C. overnight. After the reaction was completed, water (10 mL) was added thereto. The mixture thus obtained was extracted with EtOAc (10 mL×2). The combined organic phase was dried over MgSO₄, filtered and evaporated under vacuum. The residue was purified by prep. reverse-phase HPLC (0.1% TFA added) to provide the title compound (53 mg, 21%) as a white solid.

¹H NMR (400 MHz, CD₃OD): δ 6.99 (s, 1H), 6.69 (s, 1H), 3.63 (dd, J=6.0, 3.2 Hz, 1H), 3.22 (dd, J=19.2, 6.4 Hz, 1H), 3.05 (dd, J=13.2, 3.6 Hz, 1H), 2.85 (d, J=19.2 Hz, 1H), 2.75 (td, J=13.2, 3.6 Hz, 1H), 2.38 (d, J=12.4 Hz, 1H), 1.85 (dt, J=12.4, 3.2 Hz, 1H), 1.79-1.69 (m, 2H), 1.56-1.31 (m, 15H), 1.20-1.09 (m, 1H).

MH+ 300.

The following compound of Example 105 was obtained by repeating the procedure of Example 104.

### Example 105

### Preparation of (+)-3-Hydroxy-2-(1-methylcyclohexyl)morphinan TFA salt

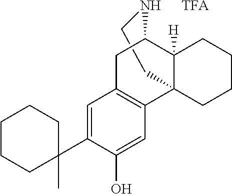

¹H NMR (400 MHz, CD₃OD): δ 7.03 (s, 1H), 6.71 (s, 1H), 3.65 (dd, J=5.6, 3.2 Hz, 1H), 3.24 (dd, J=18.8, 6.4 Hz, 1H), 3.08 (dd, J=13.2, 3.6 Hz, 1H), 2.88 (d, J=19.2 Hz, 1H), 2.77 (td, J=13.2, 3.6 Hz, 1H), 2.39 (d, J=9.2 Hz, 1H), 2.20-2.17 (m, 2H), 1.90-1.83 (m, 2H), 1.82-1.66 (m, 3H), 1.58-1.30 (m, 15H), 1.21-1.11 (m, 1H). MH+ 340.

### Example 106

### Preparation of (+)-3-Hydroxy-2-morpholinomorphinan TFA salt

### Step 1: Preparation of (+)-3-Benzyloxy-2-nitro-N-(benzyloxycarbonyl)morphinan

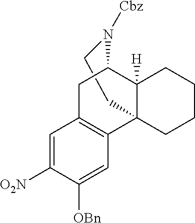

To a solution of (+)-3-hydroxy-2-nitro-N-(benzyloxycarbonyl)morphinan (42) (11.3 g, 26.7 mmol) and K₂CO₃ (7.38 g, 53.4 mmol) in DMF (100 mL) was added benzyl bromide (3.94 mL, 40.1 mmol). The resulting reaction mixture was heated at 70° C. overnight and evaporated to remove the solvent under vacuum. The residue was poured into water (300 mL) and extracted with EtOAc (150 mL×2). The combined organic phase was dried over MgSO₄ and evaporated under vacuum. The residue was purified by flash column chromatography (Biotage SP1™) to provide the title compound (13.6 g, 99%) as a white solid.

¹H NMR (400 MHz, CDCl₃): δ 7.62 (s, 1H), 7.45-1.29 (m, 10H), 6.96 (s, 1H), 5.26-5.12 (m, 4H), 4.39 (d, J=46.0 Hz, 1H), 3.97-3.85 (m, 1H), 3.08 (td, J=18.0, 5.6 Hz, 1H), 2.74-2.54 (m, 2H), 2.20 (d, J=14.0 Hz, 1H), 1.73-1.57 (m, 3H), 1.53-1.44 (m, 2H), 1.38-1.24 (m, 3H), 1.02-0.93 (m, 2H).

MH+ 513.

### Step 2: Preparation of (+)-2-Amino-3-benzyloxy-N-(benzyloxycarbonyl)morphinan

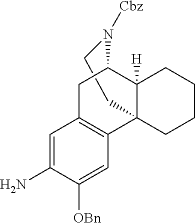

To a solution of (+)-3-benzyloxy-2-nitro-N-(benzyloxycarbonyl)morphinan obtained in step 1 (13.6 g, 26.5 mmol) and hydrazine hydrate (12.9 mL, 265 mmol) in MeOH (200 mL) was added Raney Ni (water solution, 1 mL) dropwise. The resulting reaction mixture was stirred at r.t. for 2 hr and filtered to remove the catalyst. The filtrate was evaporated under vacuum. The residue was purified by flash column chromatography (Biotage SP1™) to provide the title compound (12.5 g, 98%) as a white solid.

¹H NMR (400 MHz, CDCl₃) 7.44-7.30m, 10H), 6.69 (s, 1H), 6.45 (d, J=10.8 Hz, 1H), 5.17-5.01 (m, 4H), 4.32 (d, J=40.8 Hz, 1H), 3.96-3.82 (m, 1H), 3.72 (br s, 2H), 3.01 (td, J=17.6, 5.6 Hz, 1H), 2.73-2.52 (m, 2H), 2.20 (d, J=11.6 Hz, 1H), 1.66-1.06 (m, 10H).

MH+ 483.

### Step 3: Preparation of (+)-3-Benzyloxy-2-(2-nitrophenylamino)-N-(benzyloxycarbonyl)morphinan

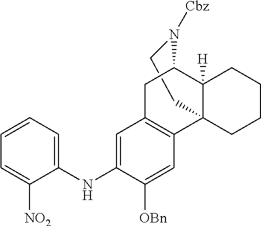

A mixture of (+)-2-amino-3-benzyloxy-N-(benzyloxycarbonyl)morphinan obtained in step 2 (2.5 g, 5.18 mmol), 1-chloro-2-nitrobenzene (1.63 g, 10.4 mmol), Pd(OAc)₂ (350 mg, 0.518 mmol), BINAP (650 mg, 1.04 mmol), and sodium t-butoxide (1.00 g, 10.4 mmol) in toluene (15 mL) was heated at 110° C. overnight and evaporated under vacuum. The residue was purified by flash column chromatography (Biotage SP1™) to provide the title compound (2.4 g, 77%) as a yellow solid.

MH+ 604.

### Step 4: Preparation of (+)-2-(2-Aminophenylamino)-3-benzyloxy-N-(benzyloxycarbonyl)morphinan

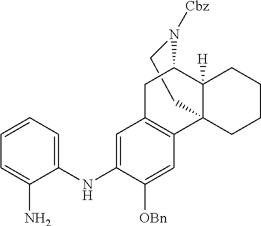

To a solution of (+)-3-benzyloxy-2-(2-nitrophenylamino)-N-(benzyloxycarbonyl)morphinan obtained in step 3 (910 mg, 1.51 mmol) and hydrazine hydrate (0.73 mL, 15.1 mmol) in MeOH (200 mL) was added Raney Ni (water solution, 1 mL) dropwise. The resulting reaction mixture was stirred at r.t. for 2 hr. and filtered to remove the catalyst. The filtrate was evaporated under vacuum. The residue was purified by flash column chromatography (Biotage SP1™) to provide the title compound (860 mg, 99%) as a white solid.

MH+ 574.

### Step 5: Preparation of (+)-2-(2-Aminophenylamino)-3-hydroxymorphinan TFA salt

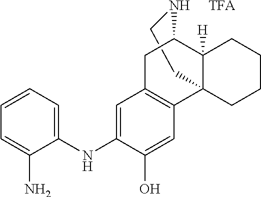

To a solution of (+)-2-(2-aminophenylamino)-3-benzyloxy-N-(benzyloxycarbonyl)morphinan obtained in step 4 (200 mg, 0.349 mmol) in DCM (10 mL) was added BBr₃ solution (1M in DCM, 1.05 mL, 1.05 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (45 mg, 28%) as a yellow solid.

¹H NMR (400 MHz, CD₃OD): δ 7.34-7.29 (m, 3H), 7.14-7.09 (m, 1H), 6.82 (s, 1H), 6.63 (s, 1H), 3.60 (q, J=2.8 Hz, 1H), 3.18-3.07 (m, 2H), 2.84-2.72 (m, 2H), 2.38 (d, J=12.0 Hz, 1H), 1.92-1.69 (m, 3H), 1.58-1.32 (m, 6H), 1.19-1.12 (m, 1H).

MH+ 350.

### Example 107

### Preparation of (+)-2-(2-Cyanophenylamino)-3-hydroxymorphinan TFA salt

### Step 1: Preparation of (+)-3-Benzyloxy-2-(2-cyanophenylamino)-N-(benzyloxycarbonyl)morphinan

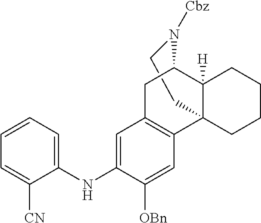

A mixture of (+)-2-amino-3-benzyloxy-N-(benzyloxycarbonyl)morphinan obtained in step 2 of Example 106 (500 mg, 1.23 mmol), 1-chloro-2-cyanobenzene (338 mg, 2.46 mmol), Pd(OAc)₂ (83 mg, 0.123 mmol), BINAP (153 mg, 0.246 mmol), and sodium t-butoxide (236 mg, 2.46 mmol) in toluene (10 mL) was heated at 110° C. overnight and evaporated under vacuum. The residue was purified by flash column chromatography (Biotage SP1™) to provide the title compound (330 mg, 53%) as a brown solid.

MH+ 508.

### Step 2: Preparation of (+)-2-(2-Cyanophenylamino)-3-hydroxymorphinan TFA salt

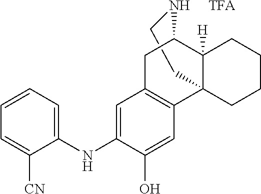

To a solution of (+)-3-benzyloxy-2-(2-cyanophenylamino)-N-(benzyloxycarbonyl)morphinan obtained in step 1 (340 mg, 0.670 mmol) in DCM (10 mL) was added BBr₃ solution (1M in DCM, 2.0 mL, 2.01 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (210 mg, 65%) as a yellow solid.

¹H NMR (400 MHz, CD₃OD): δ 7.51 (dd, J=8.0, 1.2 Hz, 1H), 7.41 (td, J=7.2, 1.6 Hz, 1H), 7.09 (d, J=8.4 Hz, 1H), 7.04 (s, 1H), 6.88 (s, 1H), 6.86 (t, J=8.0 Hz, 1H), 3.65 (q, J=2.8 Hz, 1H), 3.25 (dd, J=18.8, 6.4 Hz, 1H), 3.10 (dd, J=13.2, 3.6 Hz, 1H), 2.89-2.79 (m, 2H), 2.40 (d, J=12.4 Hz, 1H), 1.89 (d, J=12.8 Hz, 1H), 1.83-1.72 (m, 2H), 1.60-1.33 (m, 6H), 1.22-1.14 (m, 1H).

MH+ 360.

### Example 108

### Preparation of (+)-3-Hydroxy-2-(2-(methoxycarbonyl)phenylamino)morphinan TFA salt

### Step 1: Preparation of (+)-3-Benzyloxy-2-(2-(methoxycarbonyl)phenylamino)-N-(benzyloxycarbonyl)morphinan

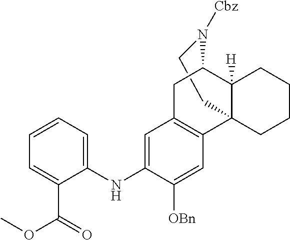

A mixture of (+)-2-amino-3-benzyloxy-N-(benzyloxycarbonyl)morphinan obtained in step 2 of Example 106 (1.50 g, 3.11 mmol), methyl 2-chlorobenzoate (0.89 mL, 6.22 mmol), Pd(OAc)₂ (209 mg, 0.311 mmol), BINAP (387 mg, 0.622 mmol), and sodium t-butoxide (299 mg, 3.11 mmol) in toluene (15 mL) was heated at 110° C. overnight and evaporated under vacuum. The residue was purified by flash column chromatography (Biotage SP1™) to provide the title compound (530 mg, 28%) as a yellow solid.

MH+ 617.

### Step 2: Preparation of (+)-3-Hydroxy-2-(2-(methoxycarbonyl)phenylamino)morphinan TFA salt

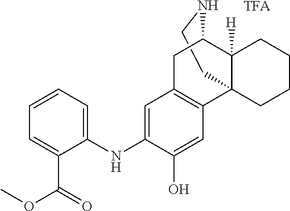

To a solution of (+)-3-benzyloxy-2-(2-(methoxycarbonyl)phenylamino)-N-(benzyloxycarbonyl)morphina n obtained in step 1 (250 mg, 0.405 mmol) in DCM (10 mL) was added BBr₃ solution (1M in DCM, 1.2 mL, 1.20 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (15 mg, 8%) as a yellow solid.

¹H NMR (400 MHz, CD₃OD): δ 7.97 (dd, J=8.0, 1.2 Hz, 1H), 7.37-7.29 (m, 2H), 7.24 (s, 1H), 6.93 (s, 1H), 6.75 (td, J=8.0, 1.2 Hz, 1H), 3.66 (q, J=2.8 Hz, 1H), 3.33-3.25 (m, 1H), 3.10 (dd, J=13.2, 3.2 Hz, 1H), 2.88 (d, J=19.2 Hz, 1H), 2.80 (td, J=13.2, 3.6 Hz, 1H), 2.51 (d, J=13.2 Hz, 1H), 1.91 (d, J=12.4 Hz, 1H), 1.84-1.72 (m, 2H), 1.66-1.33 (m, 6H), 1.24-1.15 (m, 1H).

MH+ 393.

The following compound of Example 109 was obtained by repeating the procedure of Example 108.

### Example 109

### Preparation of (+)-2-(2-Carboxyphenylamino)-3-hydroxymorphinan TFA salt

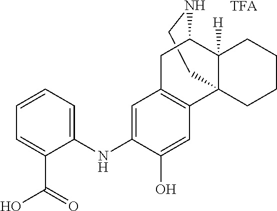

¹H NMR (400 MHz, CD₃OD): δ 7.96 (dd, J=8.0, 1.6 Hz, 1H), 7.34-7.30 (m, 1H), 7.17 (s, 1H), 7.16 (s, 1H), 6.87 (s, 1H), 6.71 (t, J=8.0 Hz, 1H), 3.64 (q, J=2.8 Hz, 1H), 3.25 (dd, J=19.2, 6.4 Hz, 1H), 3.10 (dd, J=13.2, 3.6 Hz, 1H), 2.89-2.79 (m, 2H), 2.41 (d, J=12.0 Hz, 1H), 1.89 (d, J=12.4 Hz, 1H), 1.84-1.72 (m, 2H), 1.59-1.34 (m, 6H), 1.23-1.17 (m, 1H).

MH+ 379.

### Example 110

### Preparation of (+)-3-Hydroxy-2-(2-(methanesulfonamido)phenylamino)morphinan TFA salt

### Step 1: Preparation of (+)-3-Benzyloxy-2-(2-(methanesulfonamido)phenylamino)-N-(benzyloxycarbonyl)morphinan

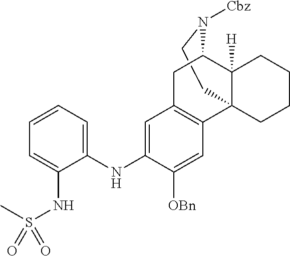

To a solution of (+)-2-(2-aminophenylamino)-3-benzyloxy-N-(benzyloxycarbonyl)morphinan obtained in step 4 of Example 106 (295 mg, 0.514 mmol) in DCM (10 mL) were added methanesulfonyl chloride (40 μL, 0.514 mmol) and TEA (0.14 mL, 1.03 mmol) at 0° C. stepwisely. The resulting reaction mixture was stirred at r.t. overnight and water (20 mL) was added thereto. The mixture thus obtained was extracted with DCM (20 mL×2). The combined organic phase was dried over MgSO₄, filtered, and evaporated under vacuum. The residue was purified by flash column chromatography (Biotage SP1™) to provide the title compound (170 mg, 51%) as a yellow solid.

MH+ 652.

### Step 2: Preparation of (+)-3-Hydroxy-2-(2-(methanesulfonamido)phenylamino)morphinan TFA salt

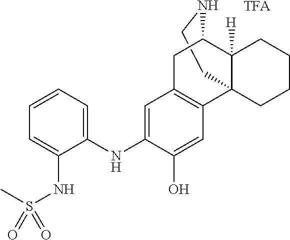

To a solution of (+)-3-benzyloxy-2-(2-(methanesulfonamido)phenylamino)-N-(benzyloxycarbonyl)morp hinan obtained in step 1 (171 mg, 0.262 mmol) in DCM (10 mL) was added BBr₃ solution (1M in DCM, 0.79 mL, 0.79 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (17 mg, 12%) as a brown solid.

¹H NMR (400 MHz, CD₃OD): δ 7.31 (dd, J=8.0, 1.2 Hz, 1H), 7.24 (dd, J=8.0, 1.2 Hz, 1H), 7.20 (td, J=7.2, 1.2 Hz, 1H), 6.93 (td, J=7.2, 1.2 Hz, 1H), 6.85 (s, 1H), 6.81 (s, 1H), 3.60 (q, J=2.8 Hz, 1H), 3.17 (dd, J=19.2, 6.4 Hz, 1H), 3.07 (dd, J=13.2, 3.2 Hz, 1H), 2.97 (s, 3H), 2.84-2.77 (m, 2H), 2.37 (d, J=10.4 Hz, 1H), 1.91-1.69 (m, 3H), 1.56-1.27 (m, 6H), 1.17-1.14 (m, 1H).

MH+ 428.

The following compounds of Examples 111 to 123 were obtained by repeating the procedure of Example 110.

### Example 111

### Preparation of (+)-2-(2-(Ethanesulfonamido)phenylamino)-3-hydroxymorphinan TFA salt

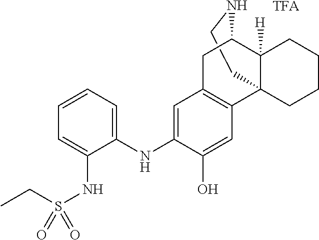

¹H NMR (400 MHz, CD₃OD): δ 7.31 (dd, J=8.0, 1.2 Hz, 1H), 7.25 (dd, J=8.0, 1.2 Hz, 1H), 7.17 (td, J=8.0, 1.2 Hz, 1H), 6.93 (td, J=8.0, 1.2 Hz, 1H), 6.81 (s, 1H), 6.80 (s, 1H), 3.60 (q, J=2.8 Hz, 1H), 3.20-3.06 (m, 4H), 2.84-2.76 (m, 2H), 2.38 (d, J=11.6 Hz, 1H), 1.88-1.85 (m, 1H), 1.80-1.72 (m, 2H), 1.57-1.31 (m, 9H), 1.20-1.12 (m, 1H).

MH+ 442.

### Example 112

### Preparation of (+)-2-(2-(Butanesulfonamido)phenylamino)-3-hydroxymorphinan TFA salt

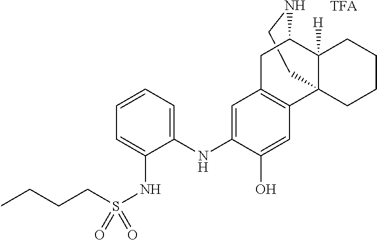

¹H NMR (400 MHz, CD₃OD): δ 7.33 (d, J=7.6 Hz, 1H), 7.29 (d, J=8.0 Hz, 1H), 7.19 (t, J=8.0 Hz, 1H), 6.95 (t, J=7.6 Hz, 1H), 6.82 (s, 1H), 6.80 (s, 1H), 3.60 (q, J=2.8 Hz, 1H), 3.16 (dd, J=19.2, 6.4 Hz, 1H), 3.08 (dd, J=12.8, 3.6 Hz, 1H), 3.02 (t, J=8.0 Hz, 2H), 2.84-2.75 (m, 2H), 2.38 (d, J=11.2 Hz, 1H), 1.86 (d, J=12.4 Hz, 1H), 1.80-1.70 (m, 4H), 1.56-1.12 (m, 8H), 0.83 (t, J=7.6 Hz, 3H).

MH+ 470.

### Example 113

### Preparation of (+)-2-(2-(Benzenesulfonamido)phenylamino)-3-hydroxymorphinan TFA salt

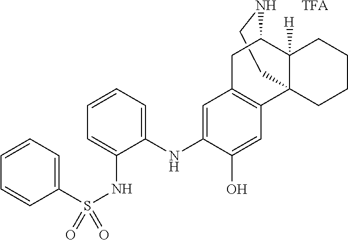

¹H NMR (400 MHz, CD₃OD): δ 7.70 (d, J=7.2 Hz, 2H), 7.54 (td, J=5.6, 1.6 Hz, 1H), 7.40 (t, J=8.0 Hz, 2H), 7.19 (dd, J=8.0, 1.6 Hz, 1H), 7.10 (td, J=7.2, 1.6 Hz, 1H), 6.82 (dd, J=7.6, 1.6 Hz, 1H), 6.77 (s, 1H), 6.73 (td, J=7.6, 1.2 Hz, 1H), 6.66 (s, 1H), 3.60 (q, J=2.8 Hz, 1H), 3.17-3.07 (m, 2H), 2.82-2.71 (m, 2H), 2.38 (d, J=7.6 Hz, 1H), 1.87-1.72 (m, 3H), 1.58-1.37 (m, 6H), 1.20-1.17 (m, 1H).

MH+ 490.

### Example 114

### Preparation of (+)-3-Hydroxy-2-(4-(methanesulfonamido)phenylamino)morphinan TFA salt

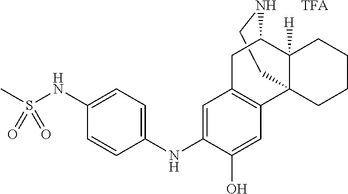

¹H NMR (400 MHz, CD₃OD): δ 7.13 (dt, J=9.2, 2.4 Hz, 1H), 7.03 (dt, J=8.8, 2.4 Hz, 1H), 6.99 (s, 1H), 6.79 (s, 1H), 3.61 (q, J=2.8 Hz, 1H), 3.20 (dd, J=19.2, 6.4 Hz, 1H), 3.08 (dd, J=13.2, 3.2 Hz, 1H), 2.88 (s, 3H), 2.85-2.77 (m, 2H), 2.38 (d, J=11.6 Hz, 1H), 1.88-1.71 (m, 3H), 1.57-1.34 (m, 6H), 1.23-1.14 (m, 1H).

MH+ 428.

### Example 115

### Preparation of (+)-3-Hydroxy-2-(2-(pivalamido)phenylamino)morphinan TFA salt

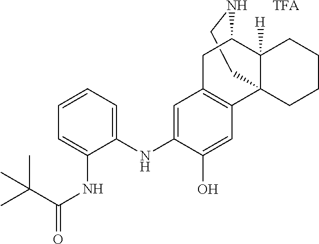

¹H NMR (400 MHz, CD₃OD): δ 7.54 (dd, J=8.0, 1.2 Hz, 1H), 7.28 (dd, J=8.0, 1.2 Hz, 1H), 7.18 (td, J=7.6, 1.6 Hz, 1H), 7.08 (td, J=7.6, 1.6 Hz, 1H), 6.76 (s, 1H), 6.41 (s, 1H), 3.56 (q, J=2.8 Hz, 1H), 3.11-3.03 (m, 2H), 2.75-2.66 (m, 2H), 2.36 (d, J=10.4 Hz, 1H), 1.84-1.68 (m, 3H), 1.56-1.34 (m, 7H), 1.15 (s, 9H).

MH+ 434.

### Example 116

### Preparation of (+)-2-(2-(Acetamido)phenylamino)-3-hydroxymorphinan TFA salt

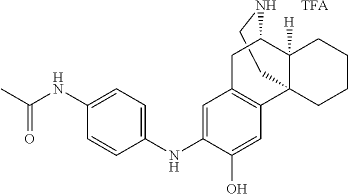

¹H NMR (400 MHz, CD₃OD): δ 7.37 (dt, J=8.8, 3.2 Hz, 2H), 7.03 (dt, J=8.8, 3.2 Hz, 2H), 6.96 (s, 1H), 6.78 (s, 1H), 3.61 (q, J=2.8 Hz, 1H), 3.19 (dd, J=19.2, 6.4 Hz, 1H), 3.07 (dd, J=13.2, 3.2 Hz, 1H), 2.84-2.77 (m, 2H), 2.37 (d, J=10.8 Hz, 1H), 2.08 (s, 3H), 1.87-1.71 (m, 3H), 1.56-1.36 (m, 6H), 1.22-1.14 (m, 1H).

MH+ 392.

### Example 117

### Preparation of (+)-2-(2-(Ethoxycarbonylamino)phenylamino)-3-hydroxymorphinan TFA salt

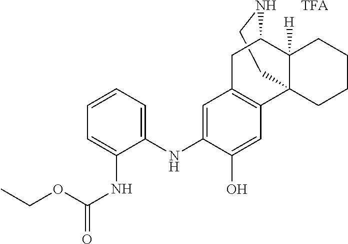

¹H NMR (400 MHz, CD₃OD): δ 7.45 (d, J=7.6 Hz, 1H), 7.21 (dd, J=7.6, 1.2 Hz, 1H), 7.10 (td, J=7.6, 1.2 Hz, 1H), 7.00 (td, J=7.6, 1.2 Hz, 1H), 6.77 (s, 1H), 6.57 (s, 1H), 4.14 (q, J=7.2 Hz, 2H), 3.58 (q, J=2.8 Hz, 1H), 3.16-3.04 (m, 2H), 2.82-2.70 (m, 2H), 2.36 (d, J=10.4 Hz, 1H), 1.84 (d, J=12.4 Hz, 1H), 1.75-1.69 (m, 2H), 1.56-1.36 (m, 6H), 1.25 (t, J=7.2 Hz, 3H), 1.17-1.04 (m, 1H).

MH+ 422.

### Example 118

### Preparation of (+)-2-(2-(Butoxycarbonylamino)phenylamino)-3-hydroxymorphinan TFA salt

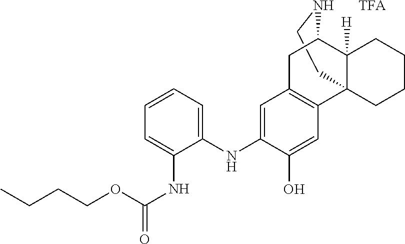

¹H NMR (400 MHz, CD₃OD): δ 7.45 (d, J=7.6 Hz, 1H), 7.21 (dd, J=8.0, 1.2 Hz, 1H), 7.10 (td, J=8.0, 1.2 Hz, 1H), 7.00 (td, J=8.0, 1.2 Hz, 1H), 6.78 (s, 1H), 6.58 (s, 1H), 4.10 (td, J=6.4, 1.6 Hz, 2H), 3.58 (q, J=2.8 Hz, 1H), 3.15-3.04 (m, 2H), 2.82-2.70 (m, 2H), 2.37 (d, J=10.8 Hz, 1H), 1.84 (d, J=12.4 Hz, 1H), 1.79-1.69 (m, 2H), 1.54-1.36 (m, 10H), 1.17-1.13 (m, 1H), 0.92 (t, J=7.6 Hz, 3H).

MH+ 450.

### Example 119

### Preparation of (+)-3-Hydroxy-2-(2-(isobutyloxycarbonylamino)phenylamino)morphinan TFA salt

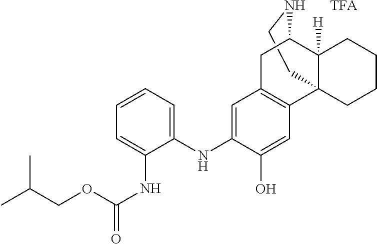

¹H NMR (400 MHz, CD₃OD): δ 7.44 (d, J=7.2 Hz, 1H), 7.22 (dd, J=8.0, 1.2 Hz, 1H), 7.10 (td, J=7.6, 1.6 Hz, 1H), 7.00 (td, J=7.6, 1.2 Hz, 1H), 6.77 (s, 1H), 6.58 (s, 1H), 3.92-3.84 (m, 2H), 3.58 (q, J=2.8 Hz, 1H), 3.15-3.04 (m, 2H), 2.81-2.70 (m, 2H), 2.37 (d, J=10.4 Hz, 1H), 1.95-1.69 (m, 4H), 1.55-1.35 (m, 6H), 1.19-1.07 (m, 1H), 0.92 (d, J=6.8 Hz, 6H).

MH+ 450.

### Example 120

### Preparation of (+)-2-(2-(Ethylureido)phenylamino)-3-hydroxymorphinan TFA salt

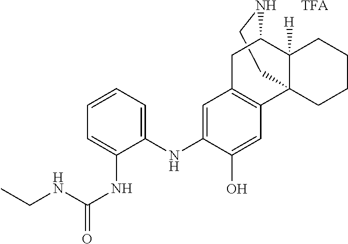

¹H NMR (400 MHz, CD₃OD): δ 7.59-7.57 (m, 1H), 7.16-7.14 (m, 1H), 7.04-7.01 (m, 2H), 6.75 (s, 1H), 6.44 (s, 1H), 3.92-3.84 (m, 2H), 3.57 (q, J=2.8 Hz, 1H), 3.16-3.07 (m, 4H), 2.81-2.68 (m, 2H), 2.37 (d, J=10.4 Hz, 1H), 1.86-1.69 (m, 4H), 1.54-1.32 (m, 6H), 1.19-1.11 (m, 1H), 1.07 (t, J=7.2 Hz, 3H).

MH+ 421.

### Example 121

### Preparation of (+)-2-(2-(Butylureido)phenylamino)-3-hydroxymorphinan TFA salt

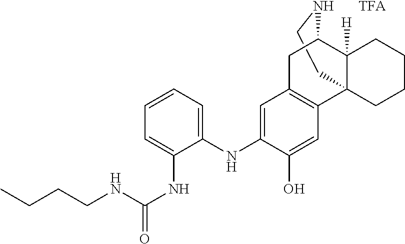

¹H NMR (400 MHz, CD₃OD): δ 7.60-7.58 (m, 1H), 7.16-7.14 (m, 1H), 7.03-7.01 (m, 2H), 6.76 (s, 1H), 6.44 (s, 1H), 3.92-3.84 (m, 2H), 3.56 (q, J=2.8 Hz, 1H), 3.15-3.06 (m, 4H), 2.80-2.68 (m, 2H), 2.36 (d, J=10.4 Hz, 1H), 1.86-1.68 (m, 3H), 1.54-1.29 (m, 10H), 1.18-1.12 (m, 1H), 0.90 (t, J=7.2 Hz, 3H).

MH+ 449.

### Example 122

### Preparation of (+)-3-Hydroxy-2-(2-(phenylureido)phenylamino)morphinan TFA salt

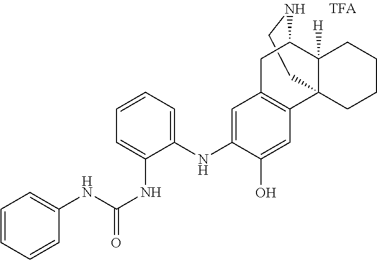

¹H NMR (400 MHz, CD₃OD): δ 7.71 (dd, J=8.0, 1.6 Hz, 1H), 7.34 (dd, J=8.4, 1.2 Hz, 2H), 7.24-7.07 (m, 5H), 6.95 (t, J=7.6 Hz, 1H), 6.74 (s, 1H), 6.29 (s, 1H), 3.45 (q, J=2.8 Hz, 1H), 3.01 (d, J=19.2, 6.4 Hz, 1H), 2.94 (dd, J=13.2, 3.2 Hz, 1H), 2.69-2.61 (m, 2H), 2.33 (d, J=11.6 Hz, 1H), 1.76-1.66 (m, 2H), 1.56-1.45 (m, 3H), 1.32-1.19 (m, 4H), 0.96-0.88 (m, 1H).

MH+ 469.

### Example 123

### Preparation of (+)-2-(2-(Butylthioureido)phenylamino)-3-hydroxymorphinan TFA salt

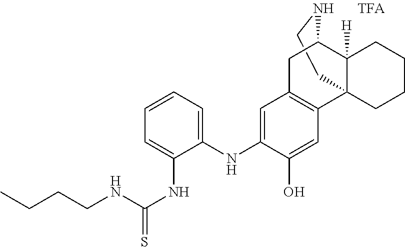

¹H NMR (400 MHz, CD₃OD): δ 7.27 (d, J=3.2 Hz, 1H), 7.20-7.14 (m, 2H), 6.93 (t, J=6.8 Hz, 1H), 6.85 (s, 1H), 6.79 (s, 1H), 3.60 (q, J=2.8 Hz, 1H), 3.49 (br s, 2H), 3.19 (dd, J=18.8, 6.0 Hz, 1H), 3.07 (dd, J=13.2, 3.2 Hz, 1H), 2.84-2.78 (m, 2H), 2.37 (d, J=12.4 Hz, 1H), 1.86 (d, J=12.4 Hz, 1H), 1.79-1.69 (m, 2H), 1.56-1.10 (m, 11H), 0.84 (t, J=7.6 Hz, 3H).

MH+ 465.

### Example 124

### Preparation of (+)-2-(4-Fluorophenyl(methyl)amino)-3-hydroxymorphinan TFA salt

### Step 1: Preparation of (+)-3-Benzyloxy-2-iodo-N-(benzyloxycarbonyl)morphinan

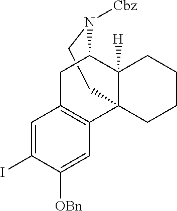

To a solution of (+)-3-hydroxy-2-iodo-N-(benzyloxycarbonyl)morphinan obtained in step 1 of Example 12 (13.5 g, 26.8 mmol) and K₂CO₃ (7.41 g, 53.6 mmol) in DMF (100 mL) was added benzyl bromide (4.8 mL, 40.2 mmol). The resulting reaction mixture was heated at 70° C. overnight and evaporated to remove the solvent under vacuum. The residue was poured into water (300 mL) and extracted with EtOAc (150 mL×2). The organic phase was dried over MgSO₄ and evaporated under vacuum. The residue was purified by flash column chromatography (Biotage SP1™) to provide the title compound (13.6 g, 99%) as a yellow solid.

MH+ 594.

### Step 2: Preparation of (+)-3-Benzyloxy-2-(4-fluorophenylamino)-N-(benzyloxycarbonyl)morphinan

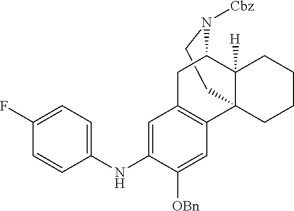

To a solution of (+)-3-benzyloxy-2-iodo-N-(benzyloxycarbonyl)morphinan obtained in step 1 (1.00 g, 1.68 mmol) in toluene (10 mL) were added 4-fluoroaniline (373 mg, 3.36 mmol), NatBuO (323 mg, 3.36 mmol), (dppf)PdCl₂.CH₂Cl₂ (54.9 mg, 0.0672 mmol), and dppf (102 mg, 0.202 mmol). The resulting reaction mixture was heated at 100° C. overnight. After the reaction was completed, water (10 mL) was added thereto. The mixture was extracted with EtOAc (15 mL×2). The combined organic phase was dried over MgSO₄, filtered, and evaporated under vacuum. The residue was purified by flash column chromatography (Biotage SP1™) to provide the title compound (540 mg, 56%) as a yellow solid.

MH+ 577.

### Step 3: Preparation of (+)-3-Benzyloxy-2-(4-fluorophenylmethyl)amino)-N-(benzyloxycarbonyl)morphinan

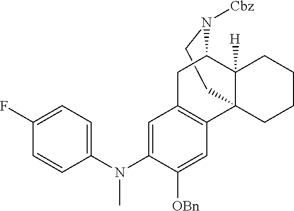

To a solution of (+)-3-benzyloxy-2-(4-fluorophenylamino)-N-(benzyloxycarbonyl)morphinan obtained in step 2 (410 mg, 0.711 mmol) in THF (10 mL) was added NaHMDS (1.4 mL, 1.42 mmol) at −78° C. slowly. After stirring of resulting reaction mixture for 30 min., methyl iodide (90 μL, 1.42 mmol) was added thereto at the same temperature. The mixture thus obtained was stirred at r.t. for 2 hrs and evaporated under vacuum. The residue was purified by flash column chromatography (Biotage SP1™) to provide the title compound (295 mg, 70%) as a yellow solid.

MH+ 591.

### Step 4: Preparation of (+)-2-(4-Fluorophenyl(methyl)amino)-3-hydroxymorphinan TFA salt

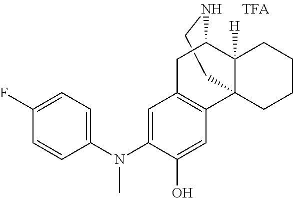

To a solution of (+)-3-benzyloxy-2-(4-fluorophenyl(methyl)amino)-N-(benzyloxycarbonyl)morphinan obtained in step 3 (295 mg, 0.499 mmol) in DCM (10 mL) was added BBr₃ solution (1M in DCM, 1.5 mL, 1.50 mmol) at 0° C. The reaction was quenched by MeOH (2 mL) and the resulting reaction mixture was evaporated under vacuum. The residue was further purified by prep. reverse-phase HPLC (0.1% TFA added) to provide the title compound (96 mg, 40%) as a white solid.

¹H NMR (400 MHz, CD₃OD): δ 6.91-6.82 (m, 4H), 6.64-6.59 (m, 2H), 3.64 (q, J=2.8 Hz, 1H), 3.26 (dd, J=19.2, 6.4 Hz, 1H), 3.14-3.09 (m, 4H), 2.86-2.76 (m, 2H), 2.43 (d, J=12.8 Hz, 1H), 1.90 (d, J=12.4 Hz, 1H), 1.84-1.73 (m, 2H), 1.62-1.34 (m, 6H), 1.21-1.11 (m, 1H).

MH+ 367.

### Example 125

### Preparation of (+)-3-Hydroxy-2-(2-(methanesulfonamido)phenyl(methyl)amino)morphinan TFA salt

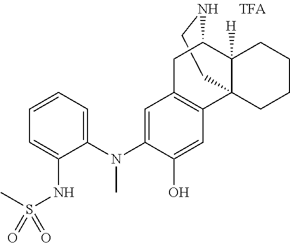

The title compound was obtained by repeating the procedure of Example 124.

¹H NMR (400 MHz, CD₃OD): δ 7.46 (dd, J=8.0, 1.6 Hz, 1H), 7.30 (dd, J=8.0, 1.6 Hz, 1H), 7.18-7.10 (m, 2H), 6.87 (s, 1H), 6.66 (s, 1H), 3.58 (q, J=2.8 Hz, 1H), 3.15-3.04 (m, 5H), 2.77-2.73 (m, 2H), 2.58 (s, 3H), 2.39 (d, J=10.4 Hz, 1H), 1.85 (d, J=12.4 Hz, 1H), 1.77-1.72 (m, 2H), 1.58-1.36 (m, 6H), 1.09-1.01 (m, 1H).

MH+ 442.

### Example 126

### Preparation of (+)-2-(2-(Dimethylamino)phenylamino)-3-hydroxymorphinan TFA salt

### Step 1: Preparation of (+)-3-Benzyloxy-2-(2-nitrophenyl(tert-butyloxylcarbonyl)amino)-N-(benzyloxycarbonyl)morphinan

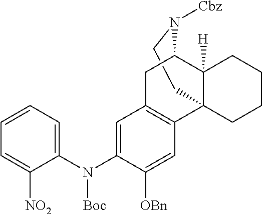

To a solution of (+)-3-benzyloxy-2-(4-nitrophenylamino)-N-(benzyloxycarbonyl)morphinan obtained in step 3 of Example 106 (9.01 g, 14.9 mmol) in THF (100 mL) were added di-tert-butyl dicarbonate (4.88 g, 22.4 mmol) and DMAP (2.18 g, 17.9 mmol) stepwisely. The resulting reaction mixture was heated at 70° C. overnight and evaporated to remove the solvent under vacuum. The residue was poured into water (300 mL) and extracted with EtOAc (150 mL×2). The combined organic phase was dried over MgSO₄ and evaporated under vacuum. The residue was purified by flash column chromatography (Biotage SP1™) to provide the title compound (7.17 g, 68%) as a red solid.

MH+ 704.

### Step 2: Preparation of (+)-(2-Aminophenyl(tert-butyloxylcarbonyl)amino)-3-benzyloxy-N-(benzyloxycarbonyl)morphinan

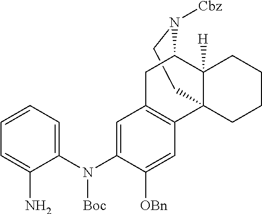

To a solution of (+)-3-benzyloxy-2-(2-nitrophenyl(tert-butyloxylcarbonyl)amino)-N-(benzyloxycarbonyl) morphinan in step 1 (7.17 g, 10.2 mmol) and hydrazine hydrate (2.5 mL, 50.9 mmol) in MeOH (200 mL) was added Raney Ni (water solution, 1 mL) dropwise. The resulting reaction mixture was stirred at r.t. for 2 hrs and filtered to remove the catalyst. The filtrate was evaporated under vacuum. The residue was purified by flash column chromatography (Biotage SP1™) to provide the title compound (6.24 g, 91%) as a yellow solid.

MH+ 674.

### Step 3: Preparation of (+)-3-Benzyloxy-2-((2-dimethylaminophenyl)(tert-butyloxylcarbonyl)amino)-N-(benzyloxycarbonyl)morphinan

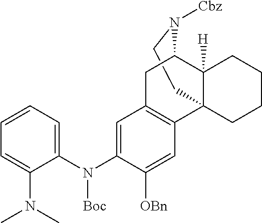

To a solution of (+)-(2-aminophenyl(tert-butyloxylcarbonyl)amino)-3-benzyloxy-N-(benzyloxycarbonyl) morphinan obtained in step 2 (220 mg, 0.326 mmol) and formalin (1.2 mL, 16.3 mmol) in DCE (10 mL) was added NaBH(OAc)₃ (415 mg, 1.96 mmol) in a portion. The resulting reaction mixture was stirred at r.t. overnight and evaporated under vacuum. The residue was poured into water (50 mL) and extracted with EtOAc (20 mL×2). The combined organic phase was dried over MgSO₄ and evaporated under vacuum. The residue was purified by flash column chromatography (Biotage SP1™) to provide the title compound (83 mg, 36%) as a yellow solid.

MH+ 702.

### Step 4: Preparation of (+)-2-(2-(dimethylamino)phenylamino)-3-hydroxymorphinan TFA salt

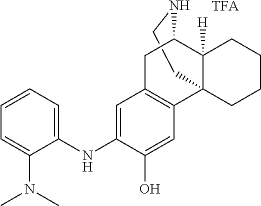

To a solution of (+)-3-benzyloxy-2-((2-dimethylaminophenyl)(tert-butyloxylcarbonyl)amino)-N-(benzyloxycarbonyl)morphinan obtained in step 3 (83 mg, 0.118 mmol) in DCM (10 mL) was added BBr₃ solution (1M in DCM, 0.4 mL, 0.40 mmol) at 0° C. The reaction was quenched by MeOH (2 mL) and the resulting reaction mixture was evaporated under vacuum. The residue was purified by prep. reverse-phase HPLC (0.1% TFA added) to provide the title compound (15 mg, 26%) as a brown solid. ¹H NMR (400 MHz, CD₃OD): δ 7.74 (dd, J=8.0, 1.2 Hz, 1H), 7.44 (td, J=7.6, 1.2 Hz, 1H), 7.38-7.33 (m, 2H), 6.81 (s, 1H), 6.55 (s, 1H), 3.60 (q, J=2.8 Hz, 1H), 3.24 (s, 6H), 3.18-3.08 (m, 2H), 2.83-2.67 (m, 2H), 2.37 (d, J=12.0 Hz, 1H), 1.89-1.68 (m, 3H), 1.58-1.28 (m, 6H), 1.18-1.09 (m, 1H).

MH+ 378.

The following compounds of Examples 127 to 131 were obtained by repeating the procedure of Example 126.

### Example 127

### Preparation of (+)-3-Hydroxy-2-(2-(methylamino)phenylamino)morphinan TFA salt

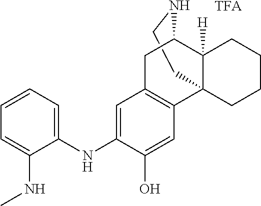

¹H NMR (400 MHz, CD₃OD): δ 7.35 (d, J=8.0 Hz, 1H), 7.30-7.27 (m, 2H), 7.23-7.18 (m, 1H), 6.82 (s, 1H), 6.54 (s, 1H), 3.60 (q, J=2.8 Hz, 1H), 3.17-3.06 (m, 2H), 3.02 (s, 3H), 2.82-2.74 (m, 2H), 2.38 (d, J=12.4 Hz, 1H), 1.87 (d, J=12.4 Hz, 1H), 1.78-1.69 (m, 2H), 1.58-1.37 (m, 6H), 1.16-1.12 (m, 1H).

MH+ 364.

### Example 128

### Preparation of (+)-3-Hydroxy-2-(2-(piperidin-1-yl)phenylamino)morphinan TFA salt

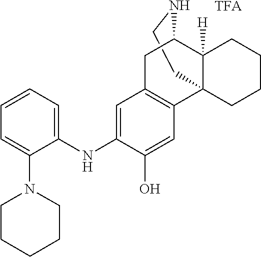

¹H NMR (400 MHz, CD₃OD): δ 7.60 (d, J=8.4 Hz, 1H), 7.36 (d, J=3.6 Hz, 2H), 7.24-7.20 (m, 1H), 6.84 (s, 1H), 6.78 (s, 1H), 3.63 (q, J=2.8 Hz, 1H), 3.53 (t, J=4.8 Hz, 4H), 3.19 (dd, J=19.2, 6.4 Hz, 1H), 3.11 (dd, J=13.2, 3.2 Hz, 1H), 2.85-2.78 (m, 2H), 2.39 (d, J=12.8 Hz, 1H), 2.04-1.98 (m, 4H), 1.89 (d, J=12.4 Hz, 1H), 1.83-1.72 (m, 4H), 1.60-1.33 (m, 6H), 1.18-1.14 (m, 1H).

MH+ 418.

### Example 129

### Preparation of (+)-2-(2-(Diethylamino)phenylamino)-3-hydroxymorphinan TFA salt

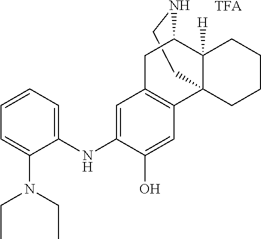

¹H NMR (400 MHz, CD₃OD): δ 7.68 (d, J=8.4 Hz, 1H), 7.52 (td, J=7.6, 1.6 Hz, 1H), 7.47-7.41 (m, 2H), 6.83 (s, 1H), 6.39 (s, 1H), 3.66 (q, J=7.2 Hz, 4H), 3.59 (q, J=2.8 Hz, 1H), 3.14-3.07 (m, 2H), 2.79-2.72 (m, 2H), 2.37 (d, J=13.2 Hz, 1H), 1.87 (d, J=12.4 Hz, 1H), 1.82-1.69 (m, 2H), 1.59-1.30 (m, 7H), 1.15 (t, J=7.2 Hz, 6H).

MH+ 406.

### Example 130

### Preparation of (+)-2-(2-(Ethylamino)phenylamino)-3-hydroxymorphinan TFA salt

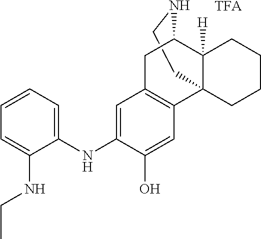

¹H NMR (400 MHz, CD₃OD): δ 7.40-7.30 (m, 3H), 7.22-7.18 (m, 1H), 6.83 (s, 1H), 6.59 (s, 1H), 3.60 (q, J=2.8 Hz, 1H), 3.45 (q, J=7.2 Hz, 2H), 3.17-3.07 (m, 2H), 2.83-2.72 (m, 2H), 2.38 (d, J=12.4 Hz, 1H), 1.88 (d, J=12.0 Hz, 1H), 1.82-1.69 (m, 2H), 1.59-1.37 (m, 6H), 1.31 (t, J=7.2 Hz, 3H), 1.18-1.09 (m, 1H).

MH+ 378.

### Example 131

### Preparation of (+)-2-(2-(Ethyl(methyl)amino)phenylamino)-3-hydroxymorphinan TFA salt

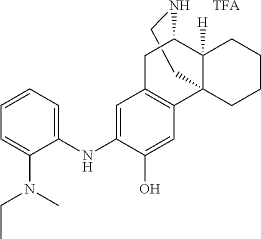

¹H NMR (400 MHz, CD₃OD): δ 7.71 (d, J=8.4 Hz, 1H), 7.48 (td, J=8.0, 1.2 Hz, 1H), 7.43-7.38 (m, 2H), 6.81 (s, 1H), 6.46 (s, 1H), 3.63-3.59 (m, 3H), 3.28 (s, 3H), 3.16-3.07 (m, 2H), 2.81-2.73 (m, 2H), 2.37 (d, J=12.8 Hz, 1H), 1.87 (d, J=12.4 Hz, 1H), 1.81-1.69 (m, 2H), 1.59-1.27 (m, 7H), 1.14 (t, J=7.2 Hz, 3H).

MH+ 392.

### Example 132

### Preparation of (+)-1-Bromo-3-hydroxy-2-(2-(methanesulfonamido)phenylamino)morphinan TFA salt

### Step 1: Preparation of (+)-3-Benzyloxy-1-bromo-2-(2-nitrophenylamino)-N-(benzyloxycarbonyl)morphinan

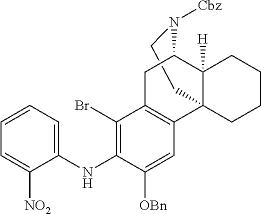

A mixture of (+)-3-benzyloxy-2-(4-nitrophenylamino)-N-(benzyloxycarbonyl)morphinan obtained in step 3 of Example 106 (960 mg, 1.59 mmol) and pyridinium tribromide (560 mg, 1.75 mmol) in THF (15 mL) was heated at 60° C. overnight and evaporated under vacuum. The residue was purified by flash column chromatography (Biotage SP1™) to provide the title compound (950 mg, 88%) as a brown solid.

MH+ 682.

### Step 2: Preparation of (+)-2-(2-Aminophenylamino)-3-benzyloxy-1-bromo-N-(benzyloxycarbonyl)morphinan

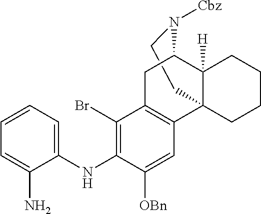

To a solution of (+)-3-benzyloxy-1-bromo-2-(2-nitrophenylamino)-N-(benzyloxycarbonyl)morphinan obtaine in step 2 (450 mg, 0.659 mmol) and hydrazine hydrate (0.16 mL, 3.30 mmol) in MeOH (50 mL) was added Raney Ni (water solution, 1 mL) dropwise. The resulting reaction mixture was stirred at r.t. for 2 hr. and filtered to remove the catalyst. The filtrate was evaporated under vacuum. The residue was purified by flash column chromatography (Biotage SP1™) to provide the title compound (267 mg, 62%) as a brown solid.

MH+ 652.

### Step 3: Preparation of (+)-3-Benzyloxy-1-bromo-2-(2-(methanesulfonamido)phenylamino)-N-(benzyloxycarbonyl)morphinan

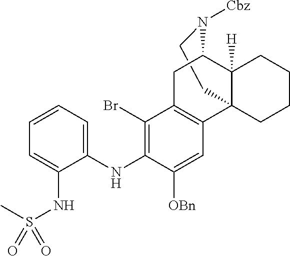

To a solution of (+)-2-(2-aminophenylamino)-3-benzyloxy-1-bromo-N-(benzyloxycarbonyl)morphinan obtained in step 2 (267 mg, 0.409 mmol) in DCM (10 mL) were added methanesulfonyl chloride (32 μL, 0.409 mmol) and TEA (86 μL, 0.614 mmol) at 0° C. stepwisely. The reaction mixture was stirred at rt overnight. Water (20 mL) was added and the mixture was extracted with DCM (20 mL×2). The combined organics were dried over MgSO₄, filtered, and evaporated under vacuum. The residue was purified by flash column chromatography (Biotage SP1™) to provide the title compound (202 mg, 68%) as a white solid.

MH+ 730.

### Step 4: Preparation of (+)-1-Bromo-3-hydroxy-2-(2-(methanesulfonamido)phenylamino)morphinan TFA salt

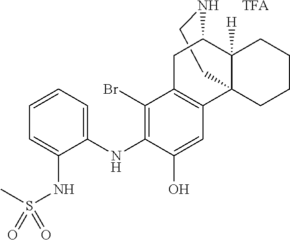

To a solution of (+)-3-benzyloxy-1-bromo-2-(2-(methanesulfonamido)phenylamino)-N-(benzyloxycarbonyl)morphinan obtained in step 3 (202 mg, 0.276 mmol) in DCM (10 mL) was added BBr₃ solution (1M in DCM, 0.83 mL, 0.83 mmol) at 0° C. After the reaction was completed, the reaction was quenched by MeOH (2 mL) evaporated under vacuum. The residue was purified by prep. reverse-phase HPLC (0.1% TFA added) to provide the title compound (111 mg, 65%) as a blue solid.

¹H NMR (400 MHz, CD₃OD): δ 7.28 (dd, J=8.0, 1.6 Hz, 1H), 7.02 (td, J=8.0, 1.6 Hz, 1H), 6.98 (s, 1H), 6.80 (td, J=7.6, 1.6 Hz, 1H), 6.34 (dd, J=8.4, 1.2 Hz, 1H), 3.76 (q, J=2.8 Hz, 1H), 3.16-3.08 (m, 2H), 3.07-2.97 (m, 4H), 2.74 (td, J=13.6, 3.6 Hz, 1H), 2.43 (d, J=13.6 Hz, 1H), 1.93 (dt, J=12.4, 3.2 Hz, 1H), 1.83 (td, J=13.6, 4.8 Hz, 1H), 1.73 (d, J=12.8 Hz, 1H), 1.67-1.30 (m, 6H), 1.15-1.07 (m, 1H).

MH+ 506.

### Example 133

### Preparation of (+)-1-Chloro-3-hydroxy-2-(2-(methanesulfonamido)phenylamino)morphinan TFA salt

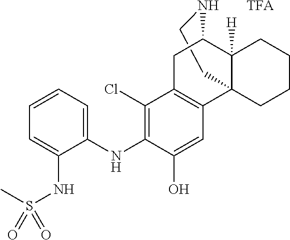

The following compound of Example 133 was obtained by repeating the procedure of Example 132.

¹H NMR (400 MHz, CD₃OD): δ 7.28 (dd, J=8.0, 1.6 Hz, 1H), 7.03 (td, J=8.0, 1.6 Hz, 1H), 6.93 (s, 1H), 6.81 (td, J=7.6, 1.2 Hz, 1H), 6.37 (dd, J=8.0, 1.2 Hz, 1H), 3.77 (q, J=2.8 Hz, 1H), 3.15-3.09 (m, 2H), 3.05-2.97 (m, 4H), 2.76 (td, J=13.6, 4.0 Hz, 1H), 2.42 (d, J=14.0 Hz, 1H), 1.94 (dt, J=12.8, 3.2 Hz, 1H), 1.83 (td, J=13.6, 4.8 Hz, 1H), 1.73 (d, J=12.4 Hz, 1H), 1.66-1.33 (m, 6H), 1.12-1.08 (m, 1H).

MH+ 462.

### Example 134

### Preparation of (+)-3-Hydroxy-2-(2-(methanesulfonamido)phenylamino)-1-methylmorphinan TFA salt

### Step 1: Preparation of (+)-3-Benzyloxy-1-methyl-2-(2-nitrophenylamino)-N-(benzyloxycarbonyl)morphinan

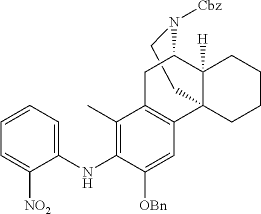

To a solution of (+)-3-benzyloxy-1-bromo-2-(2-nitrophenylamino)-N-(benzyloxycarbonyl)morphinan obtained in step 1 of Example 132 (73) (500 mg, 0.732 mmol) in 1,4-dioxane (10 mL) were added trimethylboroxine (0.21 mL, 1.46 mmol), K₂CO₃ (405 mg, 2.93 mmol), and (dppf)PdCl₂.CH₂Cl₂ (59.8 mg, 0.0732 mmol). The resulting reaction mixture was heated at 100° C. overnight. After the reaction was completed, water (10 mL) was added thereto. The resulting mixture was extracted with EtOAc (15 mL×2). The combined organic phase was dried over MgSO₄, filtered, and evaporated under vacuum. The residue was purified by flash column chromatography (Biotage SP1™) to provide the title compound (398 mg, 88%) as a red solid.

MH+ 618.

### Step 2: Preparation of (+)-2-(2-Aminophenylamino)-3-benzyloxy-1-methyl-N-(benzyloxycarbonyl)morphinan

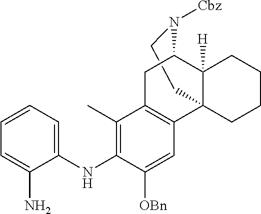

To a solution of (+)-3-benzyloxy-1-methyl-2-(2-nitrophenylamino)-N-(benzyloxycarbonyl)morphinan in step 1 (398 mg, 0.644 mmol) and hydrazine hydrate (0.16 mL, 3.22 mmol) in MeOH (50 mL) was added Raney Ni (water solution, 1 mL) dropwise. The resulting reaction mixture was stirred at r.t. for 2 hrs and filtered to remove the catalyst. The filtrate was evaporated under vacuum. The residue was further purified by flash column chromatography (Biotage SP1™) to provide the title compound (351 mg, 93%) as a white solid.

MH+ 588.

### Step 3: Preparation of (+)-3-Benzyloxy-2-(2-(methanesulfonamido)phenylamino)-1-methyl-N-(benzyloxycarbonyl)morphinan

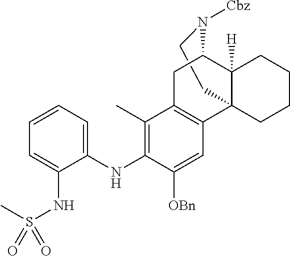

To a solution of (+)-2-(2-aminophenylamino)-3-benzyloxy-1-methyl-N-(benzyloxycarbonyl)morphinan obtained in step 2 (158 mg, 0.269 mmol) in DCM (10 mL) were added methanesulfonyl chloride (21 μL, 0.269 mmol) and TEA (56 μL, 0.404 mmol) at 0° C. stepwisely. The resulting reaction mixture was stirred at r.t. overnight. Water (20 mL) was added therteto and the mixture thus obtained was extracted with DCM (20 mL×2). The combined organic phase was dried over MgSO₄, filtered, and evaporated under vacuum. The residue was purified by flash column chromatography (Biotage SP 1 ™) to provide the title compound (174 mg, 97%) as a white solid.

MH+ 666.

### Step 4: Preparation of (+)-3-Hydroxy-2-(2-(methanesulfonamido)phenylamino)-1-methylmorphinan TFA salt

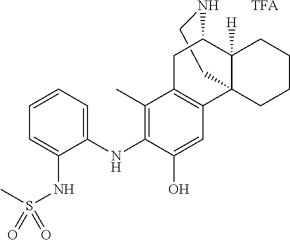

To a solution of (+)-3-benzyloxy-1-methyl-2-(2-(methanesulfonamido)phenylamino)-N-(benzyloxycarbonyl)morphinan obtained in step 3 (155 mg, 0.233 mmol) in DCM (10 mL) was added BBr₃ solution (1M in DCM, 0.7 mL, 0.70 mmol) at 0° C. The reaction was quenched by MeOH (2 mL) and the reevaporated under vacuum. The residue was purified by prep. reverse-phase HPLC (0.1% TFA added) to provide the title compound (95 mg, 65%) as a blue solid.

¹H NMR (400 MHz, CD₃OD): δ 7.24 (dd, J=8.0, 1.6 Hz, 1H), 7.01 (td, J=8.0, 1.6 Hz, 1H), 6.83 (s, 1H), 6.72 (td, J=8.0, 1.2 Hz, 1H), 6.20 (dd, J=8.0, 1.2 Hz, 1H), 3.74 (q, J=2.8 Hz, 1H), 3.13-3.04 (m, 5H), 2.82-2.75 (m, 2H), 2.43 (d, J=12.8 Hz, 1H), 2.08 (s, 3H), 1.90 (dt, J=12.4, 3.2 Hz, 1H), 1.80 (td, J=13.6, 4.4 Hz, 1H), 1.72 (d, J=11.6 Hz, 1H), 1.64-1.32 (m, 6H), 1.17-1.13 (m, 1H).

MH+ 442.

### Example 135

### Preparation of (+)-1-Cylcopropyl-3-hydroxy-2-(2-(methanesulfonamido)phenylamino)morphinan TFA salt

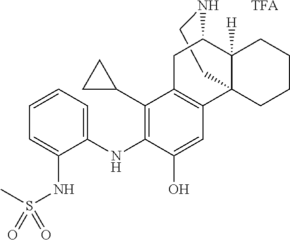

The title compound was obtained by repeating the procedure of Example 134.

¹H NMR (400 MHz, CD₃OD): δ 7.16 (d, J=15.6 Hz, 1H), 7.08 (d, J=2.0 Hz, 1H), 7.92 (dd, J=8.4, 2.4 Hz, 1H), 6.78 (s, 1H), 6.68 (s, 1H), 3.58 (q, J=2.8 Hz, 1H), 3.18-3.04 (m, 2H), 2.94 (s, 3H), 2.83-2.73 (m, 2H), 2.37 (d, J=12.8 Hz, 1H), 2.08 (s, 3H), 1.91-1.83 (m, 2H), 1.75-1.71 (m, 2H), 1.55-1.27 (m, 6H), 1.17-1.13 (m, 1H), 0.95-0.90 (m, 2H), 0.65-0.61 (m, 2H).

MH+ 468.

### Experimental Example 1

### Cell Cytotoxicity Test

HT22 cells (mouse hippocampal neuron, Salk Institute and KRIBB) were plated in a 96-well plate at a density of 3×10³ cells/well for 16 hrs before treatment. The cells were treated with five millimolar glutamate and various concentrations of the inventive compounds and incubated for 24 hrs in a growth media (DMEM with 10% FBS and 1% penicillin streptomycin). Then, the cells were treated with 3-(4,5-dimethylthiazol-2-yl)-2,5-diphenyltetrazolium bromide (MTT, Sigma®) for 4 hrs and absorbance of each well was measured with a plate reader at a wavelength of 450 nm. [Da-Qing, et. al., Anti-oxidant and anti-inflammatory activities of macelignan in murine hippocampal cell line and primary culture of rat microglia cells, BBRC, 2005, 331, 1264-1269].

Table 1 shows the results of cell cytotoxicity test of the compounds of Examples 1 to 134 and a comparative compound, 3-HM.HBr. In Table 1, EC₅₀ is neuroprotective effect against glutamate toxicity and CC₅₀ is cytotoxicity of the compounds. The EC₅₀ values were statistically analyzed using Prizm® (GraphPad Software Inc., San Diego, Calif., USA).

TABLE 1
Cell cytotoxicity test

| Example No. | EC₅₀ | | CC₅₀ | |
|---|---|---|---|---|
| 1 | 9.68 | μM | — | |
| 2 | 50 | μM | — | |
| 3 | 19.8 | μM | — | |
| 4 | 10.9 | μM | — | |
| 5 | 9.33 | μM | — | |
| 6 | 9.57 | μM | — | |
| 7 | 12.5 | μM | — | |
| 8 | 20.2 | μM | — | |
| 9 | 10.2 | μM | — | |
| 10 | 12.1 | μM | — | |
| 11 | — | | — | |
| 12 | 24.4 | μM | — | |
| 13 | 11.6 | μM | — | |
| 14 | 86.3 | μM | — | |
| 15 | — | | — | |
| 16 | >100 | μM | — | |
| 17 | 579 | nM | 12.2 | μM |
| 18 | 193 | nM | 59.0 | μM |
| 19 | 272 | nM | 8.04 | μM |
| 20 | 423 | nM | >100 | μM |
| 21 | 482 | nM | 63.5 | μM |
| 22 | 638 | nM | 80.5 | uM |
| 23 | 1.10 | μM | 49.1 | μM |
| 24 | 3.10 | μM | 14.4 | μM |
| 25 | 61.0 | nM | 1.87 | μM |
| 26 | 4.59 | nM | 2.31 | μM |
| 27 | 27.3 | nM | 1.52 | μM |
| 28 | 32.5 | nM | 1.84 | μM |
| 29 | 33.5 | nM | 19.9 | μM |
| 30 | 35.8 | nM | 7.79 | μM |
| 31 | 40.4 | nM | 7.50 | μM |
| 32 | 43.1 | nM | 14.1 | μM |
| 33 | 69.7 | nM | 1.67 | μM |
| 34 | 2.34 | μM | 41.5 | μM |
| 35 | 2.59 | μM | 52.8 | μM |
| 36 | 301 | nM | 84.5 | μM |
| 37 | 140 | nM | 14.2 | μM |
| 38 | 211 | nM | 11.8 | μM |
| 39 | 145 | nM | 13.8 | μM |
| 40 | 33.0 | nM | 7.20 | μM |
| 41 | 17.8 | nM | 6.90 | μM |
| 42 | 95.9 | nM | 11.4 | μM |
| 43 | 2.71 | nM | 10.4 | μM |
| 44 | 802 | nM | 75.0 | μM |
| 45 | 73.1 | nM | 40.9 | μM |
| 46 | 4.59 | nM | 2.31 | μM |
| 47 | 275 | nM | 9.00 | μM |
| 48 | 99.1 | nM | 2.70 | μM |
| 49 | 32.8 | nM | 1.10 | μM |
| 50 | 43.0 | nM | 1.00 | μM |
| 51 | 36.0 | nM | 1.40 | μM |
| 52 | 24.9 | nM | 1.50 | μM |
| 53 | 40.2 | nM | 1.60 | μM |
| 54 | 59.8 | nM | 1.50 | μM |
| 55 | 101 | nM | 9.20 | μM |
| 56 | 354 | nM | 12.6 | μM |
| 57 | 199 | nM | 5.50 | μM |
| 58 | 131 | nM | 1.40 | μM |
| 59 | 69.3 | nM | 1.70 | μM |
| 60 | 19.0 | nM | 1.40 | μM |
| 61 | 1.79 | μM | 50.0 | μM |
| 62 | 52.4 | nM | 1.60 | μM |
| 63 | 28.9 | nM | 10.1 | μM |
| 64 | 464 | nM | 1.90 | μM |
| 65 | 314 | nM | 1.60 | μM |
| 66 | 21.3 | nM | 2.90 | μM |
| 67 | 107 | nM | 12.5 | μM |
| 68 | 1.72 | μM | 8.50 | μM |
| 69 | 1.02 | μM | 48.0 | μM |
| 70 | 2.51 | μM | 54.6 | μM |
| 71 | 36.2 | nM | 1.60 | μM |
| 72 | 2.21 | μM | 77.3 | μM |
| 73 | 1.67 | μM | 5.57 | μM |
| 74 | 796 | nM | — | |
| 75 | 1.66 | μM | 9.40 | μM |
| 76 | 2.97 | μM | 4.4 | μM |
| 77 | 3.11 | μM | 8.30 | μM |
| 78 | 4.60 | μM | 21.0 | μM |
| 79 | 6.10 | μM | 11.0 | μM |
| 80 | 9.10 | μM | — | |
| 81 | 1.43 | μM | 11.3 | μM |
| 82 | 1.02 | μM | 28.2 | μM |
| 83 | 2.07 | μM | 51.5 | μM |
| 84 | 6.71 | μM | — | |
| 85 | 8.39 | μM | — | |
| 86 | 1.55 | μM | >100 | μM |
| 87 | 35.4 | nM | 39.5 | μM |
| 88 | 41.6 | nM | 37.2 | μM |
| 89 | 74.0 | nM | 1.94 | μM |
| 90 | 74.7 | nM | 35.0 | μM |
| 91 | 81.3 | nM | 15.0 | μM |
| 92 | 92.7 | nM | 378 | μM |
| 93 | 98.6 | nM | 13.8 | μM |
| 94 | 102 | nM | 7.55 | μM |
| 95 | 131 | nM | 66.4 | μM |
| 96 | 178 | nM | 3.49 | μM |
| 97 | 183 | nM | 67 | μM |
| 98 | 5.69 | μM | >100 | μM |
| 99 | 10.4 | μM | >100 | μM |
| 100 | 11.7 | μM | >100 | μM |
| 101 | 14.0 | μM | >100 | μM |
| 102 | 548 | nM | 29.6 | μM |
| 103 | 1.59 | μM | >100 | μM |
| 104 | 1.37 | μM | 10.0 | μM |
| 105 | 594 | nM | 2.24 | μM |
| 106 | 36.6 | nM | 21.8 | μM |
| 107 | 557 | nM | 5.80 | μM |
| 108 | 7.96 | μM | >100 | μM |
| 109 | 14.1 | μM | >100 | μM |
| 110 | 4.53 | nM | 7.55 | μM |
| 111 | 475 | nM | 36.5 | μM |
| 112 | 55.6 | nM | 20.0 | μM |
| 113 | 27.2 | nM | 3.01 | μM |
| 114 | 32.9 | nM | 11.4 | μM |
| 115 | 2.50 | μM | 53.6 | μM |
| 116 | 2.84 | μM | 36.5 | μM |
| 117 | 1.08 | μM | 36.5 | μM |
| 118 | 891 | nM | — | |
| 119 | 211 | nM | 8.7 | μM |
| 120 | 8.85 | μM | 36.5 | μM |
| 121 | >100 | μM | 23.1 | μM |
| 122 | 3.70 | μM | 33.8 | μM |
| 123 | 459 | nM | — | |
| 124 | 371 | nM | 6.40 | μM |
| 125 | 10.9 | μM | 45.9 | μM |
| 126 | 12.1 | nM | 6.10 | μM |
| 127 | 62.1 | nM | 1.30 | μM |
| 128 | 35.6 | nM | 1.60 | μM |
| 129 | 43.9 | nM | 1.60 | μM |
| 130 | 11.2 | nM | 1.60 | μM |
| 131 | 17.7 | nM | 1.50 | μM |
| 132 | 237 | nM | 10.6 | μM |
| 133 | 256 | nM | 16.5 | μM |
| 134 | 153 | nM | 53.2 | μM |
| 3-HM•HBr | 31.3 | uM | >100 | uM |

### Experimental Example 2

### ROS Measurement

HT22 cells (1×10⁴) plated in a 96-well plate were treated with 5 mM glutamate in the absence or presence of 3-HM.HBr or the compound of Example 26, and incubated for 8 hrs. After washing with PBS, cells were stained with 10 μM 2,7-dichlorodihydrofluorescein diacetate (DCFDA) in HBSS (Hank's balanced salt solution, Gibco) for 30 min in the dark. Then the cells were washed with PBS twice and extracted with 1% Triton X-100 in PBS for 10 min at 37° C. Fluorescence was recorded with the excitation wavelength of 490 nm and the emission wavelength of 525 nm (Infinite M200, TECAN) [Da-Qing, et. al., Anti-oxidant and anti-inflammatory activities of macelignan in murine hippocampal cell line and primary culture of rat microglia cells, BBRC, 2005, 331, 1264-1269]. The EC₅₀ values were analyzed statistically using Prizm® (GraphPad Software Inc., San Diego, Calif., USA).

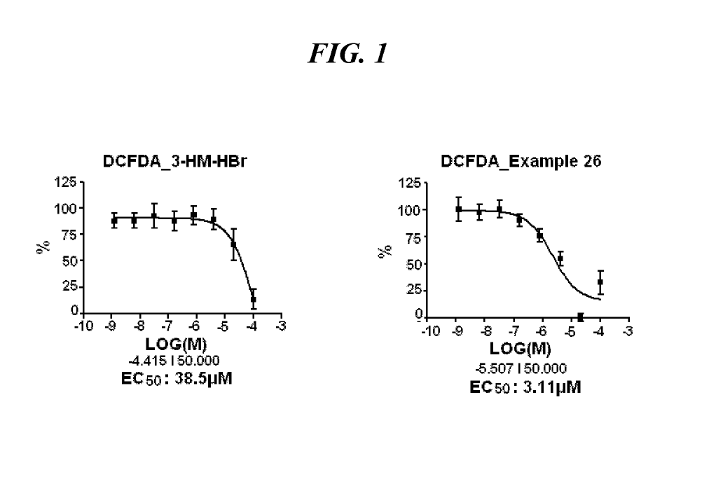

FIG. 1 shows the results of ROS measurement using DCFDA.

As can be seen from FIG. 1, the EC₅₀ of 3-HM.HBr was 38.5 μM and the EC₅₀ of the compound of Example 26 was 3.11 μM.

### Experimental Example 3

### Identifying Target Gene about Glutamate Toxicity Using RT-PCR Analysis

HT22 cells (1.5×10⁷) plated in a 100 mm dish were treated with 10 mM glutamate in the absence or presence of 3-HM.HBr or the compound of Example 26 and incubated for 18 hrs. Oligonucleotide primers used for the PCR amplification were DJ-1 (PARK7), LRRK2 (Leucine-rich repeat kinase 2), PINK1 (PTEN induced putative kinase 1), SirT1 (silent mating type information regulation 2 homolog 1 (S. cerevisiae)), SirT2 (silent mating type information regulation 2 homolog 2 (S. cerevisiae)), and others. PCR products were electrophoresed on 2% agarose gels and detected by ethidium bromide [Kumiko I. et al., The Activation of Dopamine D4 Receptors Inhibits Oxidative Stress-Induced Nerve Cell Death., J. Neurosci., 2001].

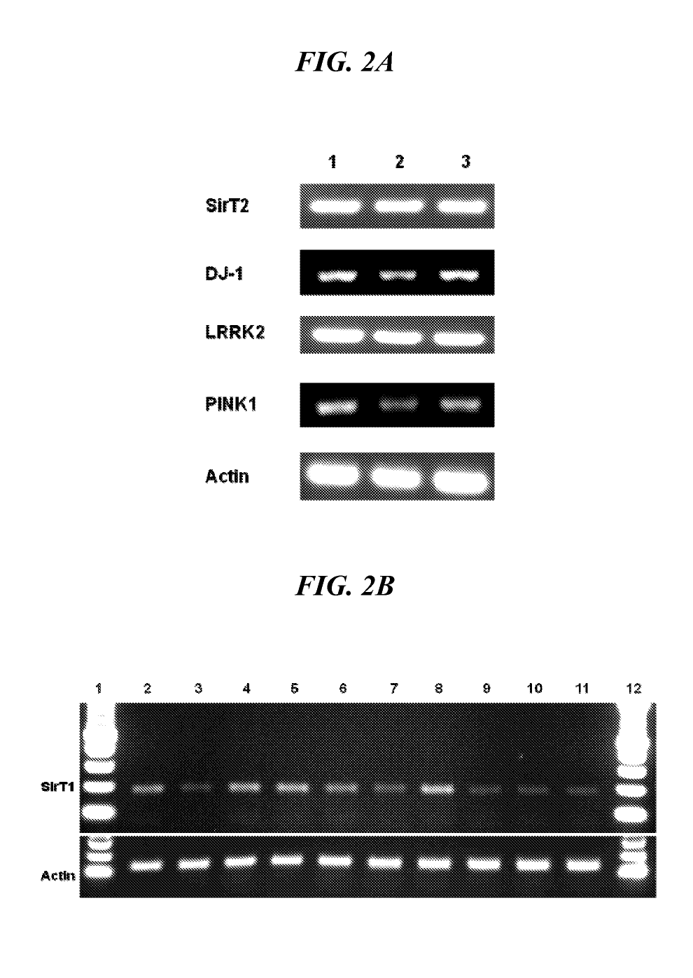

FIG. 2A and FIG. 2B show the results of reverse transcription polymerase chain reaction.

In FIG. 2A, Lane 1 is mock, Lane 2 is the group treated with 10 mM Glutamate, and Lane 3 is the group treated with 10 mM Glutamate and 100 uM 3-HM.HBr.

In FIG. 2B, Lane 1 is a DNA Ladder marker, Lane 2 is mock, Lane 3 is the group treated with 10 mM Glutamate, Lane 4 is the group treated with 10 mM Glutamate and 1 uM Example 26 compound, Lane 5 is the group treated with 10 mM Glutamate and 100 nM Example 26 compound, Lane 6 is the group treated with 10 mM Glutamate and 10 nM Example 26 compound, Lane 7 is the group treated with 10 mM Glutamate and 1 nM Example 26 compound, Lane 8 is the group treated with 10 mM Glutamate and 100 μM 3-HM.HBr, Lane 9 is the group treated with 10 mM Glutamate and 10 μM 3-HM.HBr, Lane 10 is the group treated with 10 mM Glutamate and 1 μM 3-HM.HBr, Lane 11 is the group treated with 10 mM Glutamate and 100 nM 3-HM.HBr, and Lane 12 is a DNA Ladder marker.

As shown in FIG. 2A and FIG. 2B, execept SirT1, there were no significant decreased expression levels about glutamate toxicity. Moreover, decreased SirT1 expression levels were elevated by Example 26 compound or 3-HM.HBr.

### Experimental Example 4

### Western Blotting Analysis

HT22 cells (1.5×10⁷) plated in a 100 mm dish were treated with 10 mM glutamate in the absence or presence of 3-HM.HBr or the compound of Example 26, and incubated for 18 hrs. The cells were collected by scraping in a sample buffer (3% SDS, 1% glycerol, 0.5% 2-mercaptoethanol, 0.05% bromophenol blue, and 80 mM Tris-HCl buffer, pH 6.8, with complete protease inhibitors). The resulting suspention was centrifuged and the pellets thus obtained were re-suspended in the sample buffer. The samples were heated for 3 min in boiling water, fractionated on 4-20% polyacrylamide gels, and electroblotted onto membranes. SirT1 affinity purified polyclonal antibody was used as the primary antibody. Immunoreactive bands were detected with the ECL (Amersham Pharmacia Biotech, Arlington Heights, Ill.) Western blotting detection reagents [Kumiko, I. et al., The Activation of Dopamine D4 Receptors Inhibits Oxidative Stress-Induced Nerve Cell Death., J. Neurosci., 2001, 21(16):6069-6076].

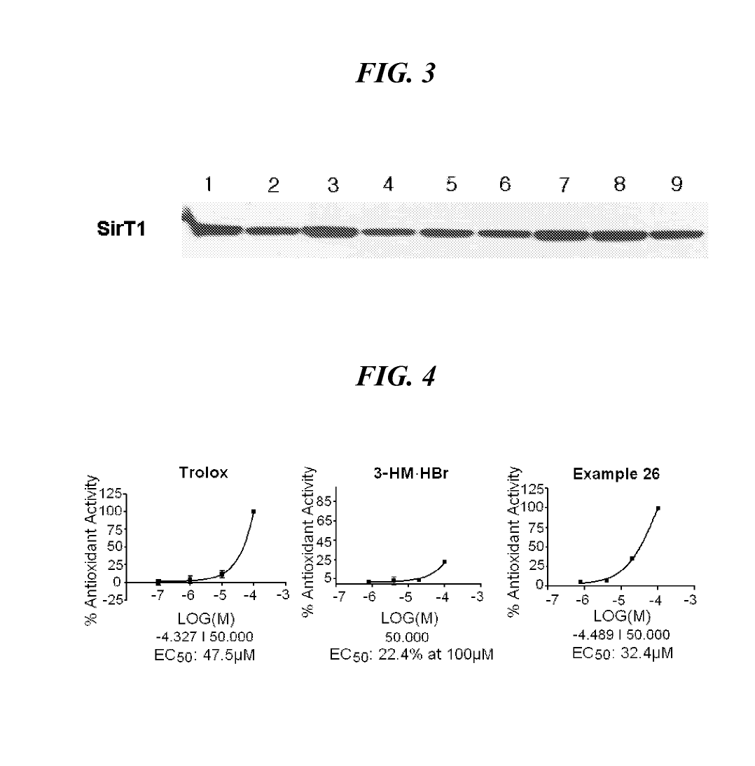

FIG. 3 shows the results of western blotting analysis.

In FIG. 3, Lane 1 is Mock, Lane 2 is the group treated with 10 mM Glutamate, Lane 3 is the group treated with 10 mM Glutamate and 100 μM 3-HM.HBr, Lane 4 is the group treated with 10 mM Glutamate and 10 μM 3-HM.HBr, Lane 5 is the group treated with 10 mM Glutamate and 1 μM 3-HM.HBr, Lane 6 is the group treated with 10 mM Glutamate and 100 nM 3-HM.HBr, Lane 7 is the group treated with 10 mM Glutamate and 1 μM Example 26 compound, Lane 8 is the group treated with 10 mM Glutamate and 100 nM Example 26 compound, Lane 9 is the group treated with 10 mM Glutamate and 10 nM Example 26 compound, and Lane 10 is the group treated with 10 mM Glutamate and 1 nM Example 26 compound.

As shown in FIG. 3, similarly RT-PCR, SirT1 expression was decreased by Glutamate toxicity and this decreasing was protected by Example 26 compound or 3-HM.HBr in Western blotting.

### Experimental Example 5

### Total Antioxidant Activity Assay

Briefly, 1 ml of reaction mixture including 2.5 μM metmyoglobin, 150 μM 2,2′-azinobis(3-ethylbenzoline 6-sulfonate), 75 μM H₂O₂, and 0.84% sample or Trolox (for standard) in PBS was incubated for 7.5 min at 30° C.; then the absorbance at 734 nm was measured. The data are normalized to 1 mM Trolox (TEAC activity) [Kumiko I. et al., The Activation of Dopamine D4 Receptors Inhibits Oxidative Stress-Induced Nerve Cell Death., J. Neurosci., 2001]. The EC50 values were analyzed statistically using Prizm® (GraphPad Software Inc., San Diego, Calif., USA).

FIG. 4 shows the results of total antioxidant activity assay.

As can be seen from FIG. 4, the EC₅₀ of Trolox was 47.5 μM, 3-HM.HBr showed 22.4% of inhibition at 100 μM; the EC₅₀ of Example 26 compound was 32.4 μM.

While the invention has been described with respect to the above specific embodiments, it should be recognized that various modifications and changes may be made to the invention by those skilled in the art which also fall within the scope of the invention as defined by the appended claims.

What is claimed is:

1. A compound of formula (I), or a pharmaceutically acceptable salt or a prodrug thereof: 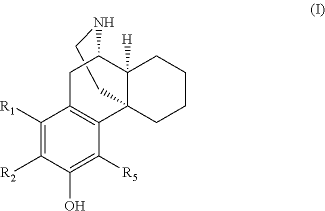
wherein,
R₁ is selected from the group consisting of hydrogen, C₁-C₃ alkyl, C₃-C₅ cycloalkyl, and halogen;
R₂ is selected from the group consisting of hydrogen; hydroxyl; mercapto; sulfanyl; sulfonyl; formyl; carboxyl; —NR₃R₄; halogen; C₁-C₁₀ alkyl; C₁-C₁₀ alkoxy; C₃-C₇ cycloalkyl; heterocycloalkyl; aryl; heteroaryl; —C₁-C₄ alkyl-Ar; and C₁-C₁₀ alkyl, C₃-C₇ cycloalkyl, heterocycloalkyl, aryl, heteroaryl, and —C₁-C₄ alkyl-Ar substituted with one or more Z groups, Ar being selected from the group consisting of phenyl, naphthyl, furyl, pyridyl, thienyl, thiazolyl, isothiazolyl, pyrazolyl, triazolyl, tetrazolyl, imidazolyl, imidazolidinyl, benzofuranyl, indolyl, thiazolidinyl, isoxazolyl, oxadiazolyl, thiadiazolyl, morpholinyl, piperidinyl, piperazinyl, pyrrolyl, and pyrimidinyl, and Z being independently selected from the group consisting of hydroxyl, C₁-C₄ alkyl, C₁-C₄ alkoxy, —(CH₂)ₘC(O)OR₃, —C(O)NR₃R₄, —CN, —(CH₂)ₙOH, —NO₂, F, Cl, Br, I, —NR₃R₄ and —NHC(O)R₃, wherein m is 0 to 4, and n is 0 to 4;
R₃ and R₄ are independently selected from the group consisting of hydrogen; C₁-C₆ alkyl; C₁-C₆ alkyl substituted with one or two R₇ groups; C₁-C₆ alkoxy; C₃-C₆ cycloalkyl; heterocycloalkyl; phenyl; heteroaryl; and C₃-C₆ cycloalkyl, heterocycloalkyl, phenyl, and heteroaryl substituted with one to three R₆ groups; or R₃ and R₄ are joined together with the N-atom to which they are attached, forming a heterocycloalkyl group or a heterocycloalkyl group substituted with one to three R₆ groups;
each R₆ is independently selected from the group consisting of hydroxyl; C₁-C₆ alkyl; C₁-C₆ alkyl substituted with one to three R₇ groups; C₁-C₆ alkoxy; halo(C₁-C₆ alkoxy); C₃-C₆ cycloalkyl; C₃-C₆ cycloalkyl substituted with one —NRₐR_{b} or pyrrolidinyl; heterocycloalkyl; phenyl; heteroaryl; —C(O)NRₐR_{b}; —C(O)R_{c}; —C(O)OR_{c}; oxo; cyano; —NRₐR_{b}; halogen; (C₁-C₆ alkyl)ureido, arylureido, and (C₁-C₆ alkylthio)ureido;
each R₇ is independently selected from the group consisting of hydroxyl; C₁-C₃ alkoxy; halogen; phenyl; cyano; —NRₐR_{b}; —C(O)NRₐR_{b}; —C(O)R_{c}; C₃-C₆ cycloalkyl; C₃-C₆ cycloalkyl substituted with one hydroxyl, heterocycloalkyl or —NRₐR_{b} group; heterocycloalkyl; heteroaryl; and heteroaryl substituted with one methyl, —NRₐR_{b} or hydroxyl;
each Rₐ is independently selected from the group consisting of hydrogen; C₁-C₃ alkyl; and C₁-C₃ alkyl substituted with one hydroxyl, methoxy, or dimethylamine; (C₁-C₄ alkyl)sulfonyl; arylsulfonyl; (C₁-C₄ alkyl)carbonyl; and (C₁-C₄ alkoxy)carbonyl;
each R_{b} is independently selected from the group consisting of hydrogen and C₁-C₃ alkyl;
each R_{c} is independently selected from the group consisting of hydrogen; C₁-C₃ alkyl; C₁-C₃ alkyl substituted with one methoxy group; phenyl; heterocycloalkyl; and heteroaryl; and
R₅ is selected from the group consisting of hydrogen and halogen, or R₅ is joined together with the adjacent hydroxyl group to form a heterocycloaryl group having two oxygens.

2. The compound of claim 1, wherein
R₁ is selected from the group consisting of hydrogen, methyl, cylcopropyl, chloro, and bromo;
R₂ is selected from the group consisting of hydroxyl, hydrogen, C₁-C₄ alkyl, C₁-C₄ haloalkyl, C₁-C₄ hydroxyalkyl, C₁-C₄ alkoxy, C₁-C₄ alkylthiol, phenylthiol, formyl, carboxyl, fluoro, chloro, bromo, iodo, (C₁-C₄ alkyl)C₃-C₇ cycloalkyl, —NR₃R₄, cyanophenyl, halophenyl, azepanyl, piperidinyl, (C₁-C₄ alkyl)piperidinyl, pyrrolidinyl, (C₁-C₄ alkyl)piperazinyl, and morpholino; and
R₃ and R₄ are independently selected from the group consisting of hydrogen, C₁-C₄ alkyl, phenyl, pyridinyl, benzodioxol, dihydrobenzo[1,4]dioxin, quinolinyl, isoquinolinyl, 1H-indazol-5-yl, 5,6,7,8-tetrahydronaphthalen-2-yl, cyclopentyl, cyclohexyl, fluorophenyl, and piperidin-1-yl.

3. The compound of claim 1, which is selected from the group consisting of:
(+)-2-Fluoro-3-hydroxymorphinan TFA salt;
(+)-4-Chloro-2-fluoro-3-hydroxymorphinan TFA salt;
(+)-4-Bromo-2-fluoro-3-hydroxymorphinan TFA salt;
(+)-2,4-Dichloro-3-hydroxymorphinan TFA salt;
(+)-4-Chloro-3-hydroxymorphinan TFA salt;
(+)-2,4-Dibromo-3-hydroxymorphinan TFA salt;
(+)-4-Bromo-3-hydroxymorphinan TFA salt;
(+)-4-Bromo-2-chloro-3-hydroxymorphinan TFA salt;
(+)-3-Hydroxy-2-iodomorphinan TFA salt;
(+)-2,3-Dihydroxymorphinan TFA salt;
(+)-3,4-(Methylenedioxy)morphinan TFA salt;
(+)-3-Hydroxy-2-methoxymorphinan TFA salt;
(+)-2-Formyl-3-hydroxymorphinan TFA salt;
((+)-3-Hydroxymorphinan)-2-carboxylic acid TFA salt;
(+)-2-(Difluoromethyl)-3-hydroxymorphinan TFA salt;
(+)-3-Hydroxy-2-(hydroxymethyl)morphinan TFA salt;
(+)-2-(Azepan-1-yl)-3-hydroxymorphinan TFA salt;
(+)-3-Hydroxy-2-(methylamino)morphinan TFA salt;
(+)-3-Hydroxy-2-(4-methylpiperidin-1-yl)morphinan TFA salt;
(+)-2-(tert-Butylamino)-3-hydroxymorphinan TFA salt;
(+)-3-Hydroxy-2-(piperidin-1-yl)morphinan TFA salt;
(+)-3-Hydroxy-2-(pyrrolidin-1-yl)morphinan TFA salt;
(+)-2-Ethylamino-3-hydroxymorphinan TFA salt;
(+)-3-Hydroxy-2-(4-methylpiperazin-1-yl)morphinan 2TFA salt;
(+)-2-(4-Chlorophenylamino)-3-hydroxymorphinan TFA salt;
(+)-3-Hydroxy-2-(4-hydroxyphenylamino)morphinan TFA salt;
(+)-2-(3,5-Dimethylphenylamino)-3-hydroxymorphinan TFA salt;
(+)-3-Hydroxy-2-(4-methylphenylamino)morphinan TFA salt;
(+)-2-(4-Fluorophenylamino)-3-hydroxymorphinan TFA salt;
(+)-3-Hydroxy-2-(phenylamino)morphinan TFA salt;
(+)-3-Hydroxy-2-(4-methoxyphenylamino)morphinan TFA salt;
(+)-2-(4-Aminophenyamino)-3-hydroxymorphinan TFA salt;
(+)-2-(4-Bromophenylamino)-3-hydroxymorphinan TFA salt;
(+)-3-Hydroxy-2-(pyridin-2-ylamino)morphinan 2TFA salt;
(+)-3-Hydroxy-2-(4-(trifluoromethyl)phenylamino)morphinan TFA salt;
(+)-3-Hydroxy-2-((3,4-methylendioxy)phenylamino)morphinan TFA salt;
(+)-2-((3,4-Ethylenedioxy)phenylamino)-3-hydroxymorphinan TFA salt;
(+)-2-(2-Fluorophenylamino)-3-hydroxymorphinan TFA salt;
(+)-2-(3-Fluorophenylamino)-3-hydroxymorphinan TFA salt;
(+)-2-(2,4-Dimethoxyphenylamino)-3-hydroxymorphinan TFA salt;
(+)-3-Hydroxy-2-(2-methylphenylamino)morphinan TFA salt;
(+)-3-Hydroxy-2-(2-methoxyphenylamino)morphinan TFA salt;
(+)-3-Hydroxy-2-(2-hydroxyphenylamino)morphinan TFA salt;
(+)-3-Hydroxy-2-(3-hydroxyphenylamino)morphinan TFA salt;
(+)-2-(2,4-Dihydroxyphenylamino)-3-hydroxymorphinan TFA salt;
(+)-2-(4-Hydroxyphenylamino)-3-hydroxymorphinan TFA salt;
(+)-2-(2,6-Dihydroxyphenylamino)-3-hydroxymorphinan TFA salt;
(+)-2-(2-Chlorophenylamino)-3-hydroxymorphinan TFA salt;
(+)-2-(2-Ethylphenylamino)-3-hydroxymorphinan TFA salt;
(+)-3-Hydroxy-2-(2-isopropylphenylamino)morphinan TFA salt;
(+)-2-(2-t-Butylphenylamino)-3-hydroxymorphinan TFA salt;
(+)-3-Hydroxy-2-(2-(trifluoromethyl)phenylamino)morphinan TFA salt;
(+)-2-(4-Ethylphenylamino)-3-hydroxymorphinan TFA salt;
(+)-3-Hydroxy-2-(4-isopropylphenylamino)morphinan TFA salt;
(+)-2-(3-Chloro-2-hydroxyphenylamino)-3-hydroxymorphinan TFA salt;
(+)-2-(5-Fluoro-2-hydroxyphenylamino)-3-hydroxymorphinan TFA salt;
(+)-2-(3-Fluoro-2-hydroxyphenylamino)-3-hydroxymorphinan TFA salt;
(+)-3-Hydroxy-2-(4-(trifluoromethoxy)phenylamino)morphinan TFA salt;
(+)-3-Hydroxy-2-(2-(trifluoromethoxy)phenylamino)morphinan TFA salt;
(+)-2-(Biphenyl-2-ylamino)-3-hydroxymorphinan TFA salt;
(+)-2-(2-Carbamoylphenylamino)-3-hydroxymorphinan TFA salt;
(+)-2-(2-Benzylphenylamino)-3-hydroxymorphinan TFA salt;
(+)-2-(3,4-Dimethoxyphenylamino)-3-hydroxymorphinan TFA salt;
(+)-2-(2,5-Dichlorophenylamino)-3-hydroxymorphinan TFA salt;
(+)-2-(3,4-Dichlorophenylamino)-3-hydroxymorphinan TFA salt;
(+)-3-Hydroxy-2-(quinolin-8-ylamino)morphinan TFA salt;
(+)-3-Hydroxy-2-(isoquinolin-5-ylamino)morphinan TFA salt;
(+)-3-Hydroxy-2-(quinolin-6-ylamino)morphinan TFA salt;
(+)-3-Hydroxy-2-((1H-indazol-5-yl)amino)morphinan TFA salt;
(+)-3-Hydroxy-2-((1H-indazol-5-yl)amino)morphinan TFA salt;
(+)-3-Hydroxy-2-((5,6,7,8-tetrahydronaphthalen-2-yl)amino)morphinan TFA salt;
(+)-3-Hydroxy-2-methylthiomorphinan TFA salt;
(+)-3-Hydroxy-2-phenylthiomorphinan TFA salt;
(+)-2-(4-Chlorophenyl)-3-hydroxymorphinan TFA salt;
(+)-3-Hydroxy-2-(4-methylphenyl)morphinan TFA salt;
(+)-2-(2,4-Dichlorophenyl)-3-hydroxymorphinan TFA salt;
(+)-2-(4-Fluorophenyl)-3-hydroxymorphinan TFA salt;
(+)-2-(3-Cyanophenyl)-3-hydroxymorphinan TFA salt;
(+)-3-Hydroxy-2-(4-trifluorophenyl)morphinan TFA salt;
(+)-3-Hydroxy-2-phenylmorphinan TFA salt;
(+)-3-Hydroxy-2-isobutylmorphinan TFA salt;
(+)-3-Hydroxy-2-propylmorphinan TFA salt;
(+)-2-Butyl-3-hydroxymorphinan TFA salt;
(+)-2-Ethyl-3-hydroxymorphinan TFA salt;
(+)-3-Hydroxy-2-methylmorphinan TFA salt;
(+)-3-Hydroxy-2-morpholinomorphinan TFA salt;
(+)-3-Hydroxy-2-isopropylaminomorphinan TFA salt;
(+)-3-Hydroxy-2-propylaminomorphinan TFA salt;
(+)-2-(Heptan-4-ylamino)-3-hydroxymorphinan TFA salt;
(+)-2-Butylamino-3-hydroxymorphinan TFA salt;
(+)-3-Hydroxy-2-(1-phenylethylamino)morphinan TFA salt;
(+)-2-Cyclopentylamino-3-hydroxymorphinan TFA salt;
(+)-2-Cyclohexylamino-3-hydroxymorphinan TFA salt;
(+)-2-Cycloheptylamino-3-hydroxymorphinan TFA salt;
(+)-2-(sec-Butylamino)-3-hydroxymorphinan TFA salt;
(+)-2-(Dipropylamino)-3-hydroxymorphinan TFA salt;
(+)-3-Hydroxy-2-(3-trifluoropropylamino)morphinan TFA salt;
(+)-2-(Dimethylamino)-3-hydroxymorphinan TFA salt;
(+)-2-(2-Ethoxyethylamino)-3-hydroxymorphinan TFA salt;
(+)-3-Hydroxy-2-(2-hydroxyethylamino)morphinan TFA salt;
(+)-2-(Diethylamino)-3-hydroxymorphinan TFA salt;
(+)-3-Hydroxy-2-(methylpropylamino)morphinan TFA salt;
(+)-2-(Ethylmethylamino)-3-hydroxymorphinan TFA salt;
(+)-2-tert-Butyl-3-hydroxymorphinan TFA salt;
(+)-3-Hydroxy-2-(1-methylcyclohexyl)morphinan TFA salt;
(+)-3-Hydroxy-2-morpholinomorphinan TFA salt;
(+)-2-(2-Cyanophenylamino)-3-hydroxymorphinan TFA salt;
(+)-3-Hydroxy-2-(2-(methoxycarbonyl)phenylamino)morphinan TFA salt;
(+)-2-(2-Carboxyphenylamino)-3-hydroxymorphinan TFA salt;
(+)-3-Hydroxy-2-(2-(methanesulfonamido)phenylamino)morphinan TFA salt;
(+)-2-(2-(Ethanesulfonamido)phenylamino)-3-hydroxymorphinan TFA salt;
(+)-2-(2-(Butanesulfonamido)phenylamino)-3-hydroxymorphinan TFA salt;
(+)-2-(2-(Benzenesulfonamido)phenylamino)-3-hydroxymorphinan TFA salt;
(+)-3-Hydroxy-2-(4-(methanesulfonamido)phenylamino)morphinan TFA salt;
(+)-3-Hydroxy-2-(2-(pivalamido)phenylamino)morphinan TFA salt;
(+)-2-(2-(Acetamido)phenylamino)-3-hydroxymorphinan TFA salt;
(+)-2-(2-(Ethoxycarbonylamino)phenylamino)-3-hydroxymorphinan TFA salt;
(+)-2-(2-(Butoxycarbonylamino)phenylamino)-3-hydroxymorphinan TFA salt;
(+)-3-Hydroxy-2-(2-(isobutyloxycarbonylamino)phenylamino)morphinan TFA salt;
(+)-2-(2-(Ethylureido)phenylamino)-3-hydroxymorphinan TFA salt;
(+)-2-(2-(Butylureido)phenylamino)-3-hydroxymorphinan TFA salt;
(+)-3-Hydroxy-2-(2-(phenylureido)phenylamino)morphinan TFA salt;
(+)-2-(2-(Butylthioureido)phenylamino)-3-hydroxymorphinan TFA salt;
(+)-2-(4-Fluorophenyl(methyl)amino)-3-hydroxymorphinan TFA salt;
(+)-3-Hydroxy-2-(2-(methanesulfonamido)phenyl(methyl)amino) morphinan TFA salt;
(+)-2-(2-(Dimethylamino)phenylamino)-3-hydroxymorphinan TFA salt;
(+)-3-Hydroxy-2-(2-(methylamino)phenylamino)morphinan TFA salt;
(+)-3-Hydroxy-2-(2-(piperidin-1-yl)phenylamino)morphinan TFA salt;
(+)-2-(2-(Diethylamino)phenylamino)-3-hydroxymorphinan TFA salt;
(+)-2-(2-(Ethylamino)phenylamino)-3-hydroxymorphinan TFA salt;
(+)-2-(2-(Ethyl(methyl)amino)phenylamino)-3-hydroxymorphinan TFA salt;
(+)-1-Bromo-3-hydroxy-2-(2-(methanesulfonamido)phenylamino) morphinan TFA salt;
(+)-1-Chloro-3-hydroxy-2-(2-(methanesulfonamido)phenylamino) morphinan TFA salt;
(+)-3-Hydroxy-2-(2-(methanesulfonamido)phenylamino)-1-methylmorphinan TFA salt; and
(+)-1-Cylcopropyl-3-hydroxy-2-(2-(methanesulfonamido)phenylamino) morphinan TFA salt.

4. A method for preparing the compound of claim 1 comprising the steps of:
subjecting (+)-3-hydroxymorphinan HBr salt to an amino protecting reaction to obtain a compound of formula (II);
conducting an electrophilic fluorination of the compound of formula (II) to obtaine a 2-fluoro analogue thereof; treating the compound of formula (II) with 1-hydroxy-1,2-benziodoxol-3(1H)-one 1-oxide, followed by reduction to provide corresponding o-diphenol derivatives; or conducting an electrophilic iodination of the compound of formula (II), followed by treating with NaOMe; and
carrying out hydrogenation of the resulting compound using palladium catalyst, 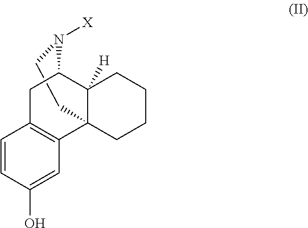
wherein, X is an amino protecting group.

5. A method for preparing the compound of claim 1 comprising the steps of:
subjecting (+)-3-hydroxymorphinan HBr salt to amino protecting reaction to obtain a compound of formula (II);
treating the compound of formula (II) with 1-hydroxy-1,2-benziodoxol-3(1H)-one 1-oxide, followed by reduction to provide corresponding o-diphenol derivatives; and
subjecting the o-diphenol derivative to a reaction with diiodomethane in the presence of a base, followed by hydrogenation using palladium catalyst, 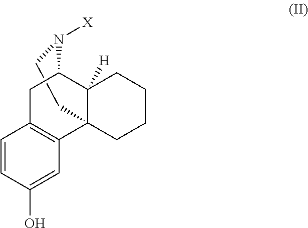
wherein, X is an amino protecting group.

6. A method for preparing the compound of claim 1 comprising the steps of:
conducting a selective ortho-formylation of the compound of formula (II) to obtain a compound of formula (III); and
subjecting the compound of formula (III) to hydrogenation using palladium catalyst, oxidation, fluorination using a fluorinating agent, or reduction using a reducing agent, followed by hydrogenation using palladium catalyst, 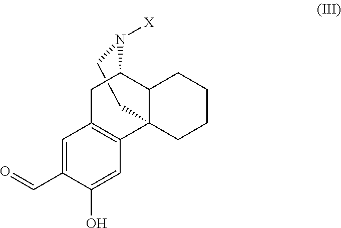
wherein, X is an amino protecting group.

7. A method for preparing the compound of claim 1 comprising the steps of:
subjecting (+)-3-hydroxymorphinan HBr salt to iodination to obtain (+)-2-iodo-3-hydroxymorphinan;
introducing an amino protecting group to (+)-2-iodo-3-hydroxymorphinan to obtain a compound of formula (IV);
conducting methylation of the compound of formula (IV) to obtain a compound of formula (V);
subjecting the compound of formula (V) to coupling reaction with cyclic amine, aniline, alkylamine, or thiol, or subjecting the compound of formula (V) to palladium-catalyzed Suzuki-Miyaura cross-coupling reaction with arylboronic acid or alkyl boronic acid to obtain a compound of formula (VI); and
carrying out demethylation of the compound of formula (VI), 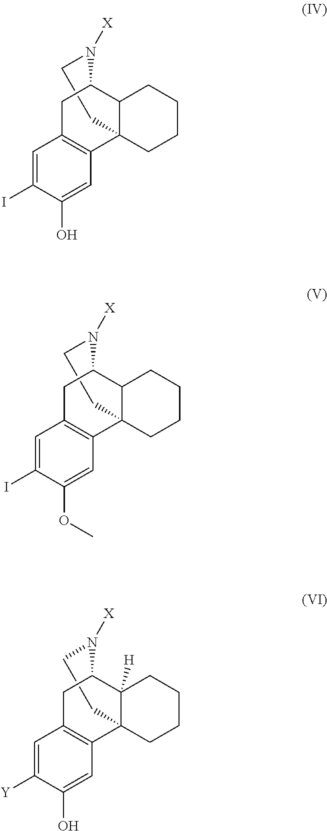
wherein, X is an amino protecting group, and Y is selected from the group consisting of —NR₃R₄; piperidinyl; mercapto; sulfanyl; aryl; C₁-C₁₀ alkyl; and piperidinyl, aryl and C₁-C₁₀ alkyl substituted with one or more Z groups, R₃, R₄ and Z having the same meanings as defined in claim 1.

8. A method for preparing the compound of claim 1 comprising the steps of:
neutralizing (+)-3-hydroxymorphinan HBr salt with a hydroxide of alkali metal to obtain (+)-3-hydroxymorphinan;
treating (+)-3-hydroxymorphinan with HNO₃ to obtain 2-nitro-3-hydroxymorphinan;
introducing an amino protecting group to 2-nitro-3-hydroxymorphinan to obtain a compound of formula (VII);
carrying out methylation of the compound of the compound of formula (VII) to obtain a compound of formula (VIII);
reducing the compound of formula (VIII) to the compound of formula (IX);
subjecting the compound of formula (IX) to a reation with 2-chloroethyl ether in the presence of a base, or a reductive alkylation with aldehyde or ketone; or conducting a amino protection reaction, alkylation, deprotection, and reductive alkylation of the compound of formula (IX) successively to obtain a compound of formula (X); and
carrying out demethylation of the compound of formula (X), 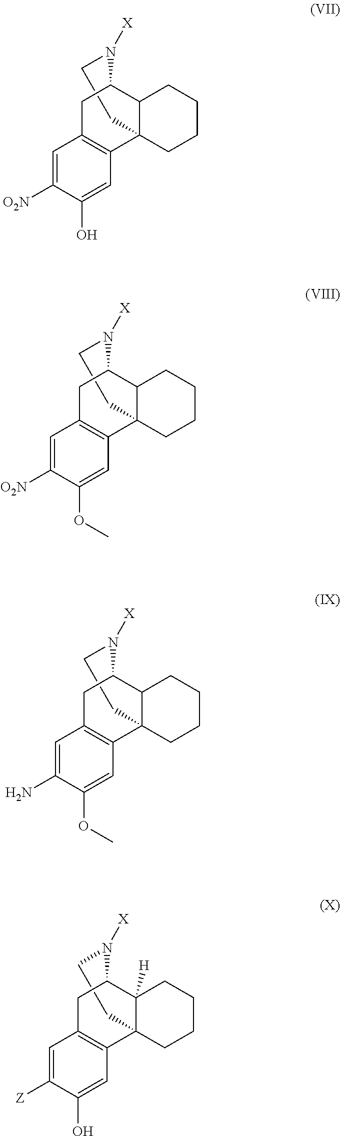
wherein, X is an amino protecting group and Z is 4-morpholinyl or —NR₃R₄, R₃ and R₄ having the same meanings as defined in claim 1.

9. A method for preparing the compound of claim 1 comprising the step of subjecting (+)-3-hydroxymorphinan HBr salt to a reaction with tertiary alcohol, a neutralization, or a bromination using bromine.

10. A pharmaceutical composition for treating or preventing a neurodegenerative disease comprising the compound of claim 1 as an active ingredient, and a pharmaceutically acceptable carrier.

11. The pharmaceutical composition of claim 4, wherein the neurodegenerative disease is Alzheimer's disease, Parkinson's disease, or Huntington's disease.

12. A method for treating or preventing a neurodegenerative disease, which comprises administering the compound of claim 1 to a mammal in need thereof.

13. A use of the compound of claim 1 for the manufacture of a medicament for preventing or treating a neurodegenerative disease.
